(19) United States
(12) Patent Application Publication
(10) Pub. No.: US 2023/0149468 A1
(43) Pub. Date: May 18, 2023

(54) PLATELET DERIVED EXTRACELLULAR VESICLES

(71) Applicant: Cellphire, Inc., Rockville, MD (US)

(72) Inventors: Ben ANTEBI, Germantown, MD (US); Keith Andrew MOSKOWITZ, Westfield, IN (US); Stephen Edward AMOS, Buckeystown, MD (US); Michael Alexander MATHEWS, Arlington, VA (US); Benjamin J. KUHN, Norfolk, VA (US); William Matthew DICKERSON, Washington, DC (US); Braden Carl ISHLER, Montgomery Village, MD (US); Daniel Allen SHEIK, West Lafayette, IN (US)

(73) Assignee: Cellphire, Inc., Rockville, MD (US)

(21) Appl. No.: 18/055,767

(22) Filed: Nov. 15, 2022

Related U.S. Application Data
(63) Continuation of application No. PCT/US2021/032783, filed on May 17, 2021.
(60) Provisional application No. 63/025,642, filed on May 15, 2020.

(51) Int. Cl. A61K 35/19

(52) U.S. Cl. CPC A61K 35/19

(57) ABSTRACT

Provided herein are methods and compositions using extracellular vesicles derived from fresh, frozen, or lyophilized platelets.

### CROSS-REFERENCE TO RELATED APPLICATIONS

This application is a continuation of International Patent Application No. PCT/US2021/032783 filed on May 17, 2021, which claims benefit of U.S. Provisional Patent Application No. 63/025,642, filed on May 15, 2020. Each of the aforementioned applications is incorporated herein by reference in its entirety.

### BACKGROUND

Provided herein are compositions and methods for use of extracellular vesicles derived from platelets, platelet derivatives, or lyophilized platelets as biological carrier of cargo, such as, for example, drugs for the therapeutic treatment of hemostasis, wound healing and disease (e.g., cancer), also referred to herein as loaded extracellular vesicles derived from platelets, stabilized platelet-derived extracellular vesicles, or lyophilized extracellular vesicles. Also provided herein, are methods of preparing stabilized extracellular vesicles from fresh, frozen, and cold stored platelets, and/or lyophilized platelet-derived extracellular vesicles.

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Unactivated platelets, which are also commonly referred to as thrombocytes, are small, often irregularly-shaped (e.g., discoidal or ovoidal) megakaryocyte-derived components of blood that are involved in the clotting process. They aid in protecting the body from excessive blood loss due not only to trauma or injury, but to normal physiological activity as well. Platelets are considered crucial in normal hemostasis, providing the first line of defense against blood escaping from injured blood vessels. Platelets generally function by adhering to the lining of broken blood vessels, in the process becoming activated, changing to an amorphous shape, and interacting with components of the clotting system that are present in plasma or are released by the platelets themselves or other components of the blood. Purified platelets have found use in treating subjects with low platelet count (thrombocytopenia) and abnormal platelet function (thrombasthenia). Concentrated platelets are often used to control bleeding after injury or during acquired platelet function defects or deficiencies, for example those occurring during surgery and those due to the presence of platelet inhibitors.

Platelet activation can trigger several cellular responses, including the release of extracellular vesicles. Extracellular vesicle is a general term given to a population of particles released from cells that are delimited by a bi-lipid bilayer and cannot replicate. Extracellular vesicles derived from platelets are classified into two main types: exosomes and microvesicles, which vary in size and are described further herein.

Platelet-derived extracellular vesicles have been shown to possess similar procoagulant activity as platelets themselves. In addition to a conventional role in hemostasis, platelet-derived extracellular vesicles may also participate in cell-to-cell communication through transfer or bioactive factors to recipient cells. Thus, platelet-derived extracellular vesicles may be advantageous over platelets, which are susceptible to short shelf life (e.g., mean of 2-3 days), exhibit diminished cell function over time (e.g., storage lesion), and have the potential to elicit transfusion-related acute lung injury (See, Msy, N., et al., Platelet Storage Lesions: What More Do We Know Now? Transfus Med Rev. pii: S0887-7963(17)30189-X. doi: 10.1016/j.tmrv.2018.04.001 (2018) and Holness, L., et al., Fatalities caused by TRALI. Transfus Med Rev. 18(3):184-188, doi:10.1016/j.tmrv.2004.03 (2004)).

### SUMMARY OF THE INVENTION

Described herein are compositions and methods for stabilizing platelet derived vesicles (e.g., extracellular vesicles) which can be used for therapeutics applications, such as, for example, hemostasis, wound healing, and cargo (e.g., drug) delivery applications for diseases (e.g., cancer) in addition to, or alternatively, to standard liquid stored platelets.

Provided herein is a a method of preparing stabilized platelet-derived extracellular vesicles, the method including generating a population of extracellular vesicles from platelets by allowing the platelets to shed a population of extracellular vesicles over a period of time in the range of approximately 2 days to approximately 21 days, isolating the population of extracellular vesicles and stabilizing the populations of extracellular vesicles with a loading buffer comprising a salt, a base, a loading agent, and optionally at least one organic solvent, to form stabilized platelet-derived extracellular vesicles.

Also provided herein are methods of preparing stabilized platelet-derived extracellular vesicles, the method comprising: generating a population of extracellular vesicles from platelets by allowing the platelets to shed a population of extracellular vesicles over a period of time in the range of approximately 2 days to approximately 21 days; isolating the population of extracellular vesicles; and stabilizing the populations of extracellular vesicles with a loading buffer comprising a salt, a base, a loading agent, and optionally at least one organic solvent, to form stabilized platelet-derived extracellular vesicles.

Also provided herein are methods of preparing stabilized platelet-derived extracellular vesicles, the method comprising: contacting platelets with a stimulating agent to generate a population of extracellular vesicles; isolating the population of extracellular vesicles; and stabilizing the population of extracellular vesicles with a loading buffer comprising a salt, a base, at least one organic solvent, and optionally a saccharide to form stabilized platelet-derived extracellular vesicles.

In some embodiments, the stimulating agent comprises one of a chemical stimulating agent, a mechanical stimulating agent, or combinations thereof. In some embodiments, the platelets are contacted with a stimulating agent for a period of time in the range of approximately 5 minutes to approximately 24 hours. In some embodiments, the chemical stimulating agent comprises a calcium ionophore, collagen, thrombin, a hypertonic solution, or combinations thereof. In some embodiments, the mechanical stimulating agent comprises sonication.

In some embodiments, one or more stabilized platelet-derived extracellular vesicles comprises an exosome, wherein the exosome is in the range of approximately 40 nm to approximately 200 nm in diameter.

In some embodiments, one or more stabilized platelet-derived extracellular vesicles comprises a microvesicle, wherein the microvesicle is in the range of approximately 200 nm to approximately 450 nm.

In some embodiments, the method includes ultracentrifuging to the population of extracellular vesicles. In some embodiments, the method includes filtering the population of extracellular vesicles.

In some embodiments, the loading buffer comprises a stabilization agent, wherein the stabilization agent is a monosaccharide, a disaccharide, a synthetic polymer of a disaccharide, or combinations thereof. In some embodiments, the stabilization agent is sucrose, polysucrose, maltose, trehalose, glucose, mannose, or xylose.

In some embodiments, the stabilization agent comprises sucrose and trehalose. In some embodiments, the stabilization agent comprises polysucrose and trehalose. In some embodiments, the trehalose is present in the range of approximately 1% (w/v) to approximately 3% (w/v), the sucrose present in the range of approximately 5% (w/v) to approximately 10% (w/v), and the polysucrose is present in the range of approximately 5% (w/v) to approximately 10% (w/v). In some embodiments, the loading buffer comprises PBS and: (i) either 7% (w/v) sucrose or 7% (w/v) polysucrose; and (ii) 2% (w/v) trehalose.

In some embodiments, the loading buffer further comprises one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

In some embodiments, the platelets are isolated prior to the contacting step.

In some embodiments, the platelets are pooled from a plurality of donors.

In some embodiments, the stabilized platelet-derived extracellular vesicles are generated from the group consisting of fresh platelets, stored platelets, frozen platelets, freeze-dried platelets, thrombosomes, and any combination thereof.

In some embodiments, the method includes cold storing, cryopreserving, freeze-drying, drying, thawing, rehydrating, or combinations thereof the stabilized platelet-derived extracellular vesicles.

In some embodiments, the drying step comprises freeze-drying the stabilized platelet-derived extracellular vesicles.

In some embodiments, the method includes rehydrating the stabilized platelet-derived extracellular vesicle.

In some embodiments, the platelets are contacted with an imaging agent, and wherein the stabilized platelet derived extracellular vesicle is loaded with the imaging agent.

Also provided herein is a a method of preparing stabilized platelet-derived extracellular vesicles, the method including generating a population of extracellular vesicles from platelets over a period of time in the range of approximately 2 days to approximately 21 days, isolating the population of extracellular vesicles, and stabilizing the populations of extracellular vesicles with a loading buffer comprising a salt, a base, a loading agent, and at optionally at least one organic solvent, to form stabilized platelet-derived extracellular vesicles.

Also provided herein a method of preparing stabilized platelet-derived extracellular vesicles, the method including contacting platelets with a stimulating agent to generate a population of extracellular vesicles, isolating the population of extracellular vesicles, and stabilizing the population of extracellular vesicles with a loading buffer comprising a salt, a base, a loading agent, and optionally at least one organic solvent, to form stabilized platelet derived extracellular vesicles.

Also provided herein is a method of preparting stabilized platelet-derived extracellular vesicles, the method including, contacting platelets with a stimulating agent to generate a population of extracellular vesicles, isolating the population of extracellular vesicles and stabilizing the population of extracellular vesicles with a loading buffer comprising a salt, a base, a saccharide, and optionally at least one organic solvent, to form stabilized platelet-derived extracellular vesicles, to form stabilized platelet-derived extracellular vesicles.

In some embodiments of a method of preparing stabilized platelet-derived extracellular vesicles, the stimulating agent includes one of a chemical stimulating agent, a mechanical stimulating agent, or combinations thereof. In some embodiments of a method of preparing stabilized platelet-derived extracellular vesicles, the platelets are contacted with a stimulating agent for a period of time in the range of approximately 5 minutes to approximately 24 hours.

In some embodiments of a method of preparing stabilized platelet-derived extracellular vesicles, the chemical stimulating agent comprises a calcium ionophore, collagen, thrombin, a hypertonic solution, or combinations thereof. In some embodiments of a method of preparing stabilized platelet-derived extracellular vesicles, the mechanical stimulating agent comprises sonication.

In some embodiments of a method of preparing stabilized platelet-derived extracellular vesicles, one or more platelet-derived extracellular vesicles of the population of extracellular vesicles includes an exosome. In some embodiments of a method of preparing stabilized platelet-derived extracellular vesicles, the exosome is in the range of approximately 40 nm to approximately 200 nm in diameter

In some embodiments of a method of preparing stabilized platelet-derived extracellular vesicles, one or more platelet-derived extracellular vesicles includes a microvesicle. In some embodiments of a method of preparing stabilized platelet-derived extracellular vesicles, the microvesicle is in the range of approximately 200 nm to approximately 450 nm.

In some embodiments of a method of preparing stabilized platelet-derived extracellular vesicles, the method includes applying ultracentrifugation to the population of extracellular vesicles. In some embodiments of a method of preparing stabilized platelet-derived extracellular vesicles, the method includes applying filtration to the extracellular vesicles.

In some embodiments of a method of preparing stabilized-platelet-derived extraceullar vesilces, the loading buffer includes a stabilization agent.

In some embodiments of a method of preparing stabilized platelet-derived extracellular vesicles, the loading buffer includes a stabilization agent, where the stabilization agent is a monosaccharide, a disaccharide, a synthetic polymer of a disaccharide, or combinations thereof. In some embodiments of a method of preparing stabilized platelet-derived extracellular vesicles, the loading buffer includes the stabilization agent, where the stabilization agent is sucrose, polysucrose, maltose, trehalose, glucose, mannose, or xylose.

In some embodiments of a method of preparing stabilized platelet-derived extracellular vesicles, the loading buffer includes the stabilization agent, where the stabilization agent includes sucrose and trehalose. In some embodiments of a method of preparing stabilized platelet-derived extracellular vesicles, the loading buffer includes the stabilization agent, where the stabilization agent includes polysucrose and trehalose. In some embodiments of a method of preparing stabilized platelet-derived extracellular vesicles, the trehalose is present in the range of approximately 1% (w/v) to approximately 3% (w/v). In some embodiments of a method of preparing stabilized platelet-derived extracellular vesicles, sucrose is present in the range of approximately 5% (w/v) to approximately 10% (w/v). In some embodiments of a method of preparing stabilized platelet-derived extracellular vesicles, polysucrose is present in the range of approximately 5% (w/v) to approximately 10% (w/v). In some embodiments of a method of preparing stabilized platelet-derived extracellular vesicles, the loading buffer includes PBS and 7% (w/v) sucrose and 2% (w/v) trehalose. In some embodiments of a method of preparing stabilized platelet-derived extracellular vesicles, the loading buffer includes PBS and 7% (w/v) polysucrose and 2% (w/v) trehalose.

In some embodiments of a method of preparing stabilized platelet-derived extracellular vesicles, one or more organic solvents are selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

In some embodiments of a method of preparing stabilized platelet-derived extracellular vesicles, the platelets are isolated prior to the contacting step. In some embodiments of a method of preparing stabilized platelet-derived extracellular vesicles, the platelets are pooled from a plurality of donors prior to the contacting step.

In some embodiments of a method of preparing stabilized platelet-derived extracellular vesicles, the stabilized platelet-derived extracellular vesicles are generated from the group consisting of fresh platelets, stored platelets, frozen platelets, freeze-dried platelets, thrombosomes, and any combination thereof.

In some embodiments of a method of preparing stabilized platelet-derived extracellular vesicles, includes cold storing, cryopreserving, freeze-drying, drying, thawing, rehydrating, or combinations thereof the stabilized platelet-derived extracellular vesicles.

In some embodiments of a method of preparing stabilized platelet-derived extracellular vesicles, the drying step includes freeze-drying the stabilized platelet-derived extracellular vesicles. In some embodiments of a method of preparing stabilized platelet-derived extracellular vesicles, includes rehydrating the stabilized platelet-derived extracellular vesicle.

In some embodiments of a method of preparing stabilized platelet-derived extracellular vesicles, the platelets are further contacted with an imaging agent, where the stabilized platelet derived extracellular vesicle is loaded with the imaging agent.

Also provided herein are stabilized platelet-derived extracellular vesicles prepared by the method of any of the methods described herein.

Also provided herein is a composition including stabilized platelet derived extracellular vesicles generated by contacting platelets with a stimulating agent including at least about 50% stabilized platelet derived extracellular vesicles. In some embodiments, the composition including stabilized extracellular vesicles generated by contacting platelets with a stimulating agent including at least about 50% stabilized platelet-derived extraceulluar vesicles are generated by any of the methods described herein.

Also provided herein is a composition including stabilized platelet derived extracellular vesicles generated by contacting platelets with a stimulating agent including at least about 75% stabilized platelet derived extracellular vesicles.

Also provided herein in is a composition including stabilized platelet derived extracellular vesicles generated by contacting platelets with a stimulating agent including at least about 80% stabilized platelet derived extracellular vesicles.

Also provided herein is a composition including stabilized platelet derived extracellular vesicles generated by contacting platelets with a stimulating agent including at least about 90% stabilized platelet derived extracellular vesicles.

Also provided herein is a method of treating bleeding in a subject in need thereof, including administering a therapeutically effective amount of stabilized platelet-derived extracellular vesicles generated by contacting platelets with a stimulating agent to the subject in need thereof.

Also provided herein is a a method of treating a wound in a subject in need thereof, including administering a therapeutically effective amount of stabilized platelet-derived extracellular vesicles generated by contacting platelets with a stimulating agent to the subject in need thereof.

Also provided herein are stabilized platelet-derived extracellular vesicles prepared by any of the methods described herein treating or ameliorating regenerative medicine.

Also provided herein is a regenerative medicine method including administering stabilized platelet-derived extracellular vesicles prepared by any method described herein.

Also provided herein is a regenerative medicine method including adminstering stabilized platelet-derived extracellular vesicles, wherein the stabilized platelet-derived extracellular vesicles comprise one or more growth factors, one or more cytokines, one or more chemokines, and combinations thereof. In some embodiments of a rengenerative medicine method, the one or more growth factors comprises platelet factor 4.

Also provided herein is a method of preparing drug-loaded stabilized platelet-derived extracellular vesicles, contacting platelets with a drug and a loading buffer comprising a salt, a base, a loading agent, and optionally at least one organic solvent to form drug-loaded platelets, contacting the drug-loaded platelets with a stimulating agent to generate a population of drug-loaded extracellular vesicles, isolating the population of drug-loaded extracellular vesicles, and stabilizing the population of drug-loaded extracellular vesicles with the loading buffer, to form drug-loaded extracellular vesicles.

Also provided herein is a method of preparing drug-loaded stabilized platelet-derived extracellular vesicles, contacting platelets with a drug and a loading buffer comprising a salt, a base, a loading agent, and optionally at least one organic solvent to form drug-loaded platelets, generating a population of drug-loaded extracellular vesicles from the drug-loaded platelets by allowing the drug-loaded platelets to shed the population of extracellular vesicles over a period of time in the range of approximately 2 days to approximately 21 days, isolating the population of drug loaded extracellular vesicles, and stabilizing the population of drug-loaded extracellular vesicles with the loading buffer, to form the drug-loaded extracellular vesicles.

In some embodiments of a method of preparing drug-loaded stabilized platelet-derived extracellular vesicles, the drug and the loading buffer are contacted with the platelets sequentially in either order, or concurrently.

In some embodiments of a method of preparing drug-loaded stabilized platelet-derived extracellular vesicles, the loading buffer includes either sucrose or polysucrose and trehalose. In some embodiments of a method of preparing drug-loaded stabilized platelet-derived extracellular vesicles, the loading buffer comprises trehalose in the range from about 0.1% (w/v) to about 5% (w/v). In some embodiments of a method of preparing drug-loaded stabilized platelet-derived extracellular vesicles, the loading buffer comprises either polysucrose or sucrose in the range from about 2% (w/v) to about 10% (w/v).

In some embodiments of a method of preparing drug-loaded stabilized platelet-derived extracellular vesicles, the drug-loaded stabilized platelet-derived extracellular vesicles treat a disease in a subject in need thereof. In some embodiments of a method of preparing drug-loaded stabilized platelet-derived extracellular vesicles the disease is cancer.

In some embodiments of a method of preparing drug-loaded stabilized platelet-derived extracellular vesicles, includes cold storing, cryopreserving, freeze-drying, thawing, rehydrating, and combinations thereof the drug-loaded stabilized platelet-derived extracellular vesicles.

In some embodiments of a method of preparing drug-loaded stabilized platelet-derived extracellular vesicles, the drug-loaded stabilized platelet-derived extracellular vesicles are generated from the group consisting of drug-loaded fresh platelets, drug-loaded stored platelets, drug-loaded frozen platelets, drug-loaded freeze-dried platelets, drug-loaded thrombosomes, and any combination thereof.

### DESCRIPTION OF DRAWINGS

FIG. 1 is a graph showing surface marker expression of filtered (e.g., fresh), frozen, and lyophilized extracellular vesicles.

FIGS. 2A-B—is a graph showing loaded and unloaded extracellular vesicles' ability to generate thrombin in the presence of an agonist (2A). FIG. 2B is a graph showing extracellular vesicles' ability to generate thrombin in the absence of a stimulating reagent.

FIG. 3 is a graph showing aggregation of extracellular vesicles in the absence of an agonist.

FIG. 4 is a graph showing adhesion of extracellular vesicles under flow in vitro using the T-TAS system.

FIG. 5 is a graph showing flow cytometry data of unlabeled and CFDA-loaded extracellular vesicles throughout the stabilization process.

FIG. 6 is a graph showing protein concentration data measured in a microBCA assay of thrombosome-derived extracellular vesicles throughout the stabilization process.

FIG. 7 is a graph showing the diameter of platelet-derived extracellular vesicles prepared under different conditions signifying two distinct population consisting of smaller-sized (i.e., exosomes) and larger-sized (i.e., microparticles) vesicles.

FIG. 8 is a graph showing the presence of platelet-specific cell surface markers on platelet-derived extracellular vesicles prepared under different conditions. The figure shows high expression of CD9 when generated under the platelet additive solution (PAS) method, indicating the presence of exosomes in the extracellular vesicle population.

FIG. 9 shows percent positivity via flow cytometry of the surface markers CD41, CD9, and phosphatidylserine (PS) present on EVs generated via the following methods: 7-day aging in PAS unfiltered (EV3), ionophore filtered (EV8F), 3-day aging room temperature filtered (R3F), 6-day aging at room temperature filtered (R6F), 14-day aging at room temperate filtered (R14F), 3-day aging at 4° C. filtered (C3F), 14-day aging at 4° C. filtered (C14F), 2-day aging at room temperature then cold shocked unfiltered (CS2), and 14-day aging at room temperature then cold shocked unfiltered samples (CS14). The figure shows enhanced expression of those markers under the different conditions, suggesting higher state of extracellular activation.

FIG. 10 is a graph showing total protein content of the extracellular vesicles evaluated by the micro bicinchoninic acid (BCA) assay. The figure shows high levels of protein content in the EV product, demonstrating the potential for multiple functional effects via direct and indirect (paracrine) mechanisms.

FIG. 11 is a graph showing the total phospholipid content of extracellular vesicles using a fluorometric assay. The figure shows high phospholipid content on the extracellular vesicles, corresponding to their high level of activation.

FIG. 12 shows endogenous thrombin potential (ETP, left Y-axis) and peak thrombin (right Y-axis) of extracellular vesicles. The figure shows high levels of thrombin burst provided by the extracellular vesicle product, similar to that of Thrombosomes, signifying their capacity to contribute to primary and secondary hemostasis.

FIG. 13 is a graph showing peak thrombin generation of extracellular vesicles. The figure shows high thrombin potential of extracellular vesicles at the proposed therapeutic dose of 1×10¹³ particles/ml, but essentially no response when significantly diluted.

FIG. 14 is a graph showing thrombolux size profile scattering intensity (kHz) by radius. The figure shows significant reduction in the mean radius of the EVs in the final product due to the tangential flow filtration process.

FIG. 15 is a graph showing thrombolux size profile (scattering intensity normalized) showing % occupancy by radius.

FIG. 16 is a graph showing nano tracking analysis (NTA) size profile for various samples. The figure shows distinct peak of the final product (lyophilized) corresponding to a mean diameter around 100 nm, indicating that the majority of the EVs are exosomes.

FIG. 17 is a graph showing the total phospholipid content of extracellular vesicles using a fluorometric assay. The figure shows high phospholipid content on the extracellular vesicles, corresponding to their high level of activation

FIG. 18 shows endogenous thrombin potential (ETP, left Y-axis) and peak thrombin (right Y-axis) of extracellular vesicles. The figure shows high levels of thrombin burst provided by the extracellular vesicle product, similar to that of Thrombosomes, signifying their capacity to contribute to primary and secondary hemostasis.

FIG. 19 is a graph showing peak thrombin generation of extracellular vesicles. The figure shows high thrombin potential of extracellular vesicles at the proposed therapeutic dose of 1×1013 particles/ml, but essentially no response when significantly diluted.

FIG. 20 is a graph showing pressure (kPa) over time for various samples. The figure shows that extracellular vesicles can adhere to collagen and promote thrombus formation under shear.

FIG. 21 is a graph showing occlusion time between Octaplas and extracellular vesicles. The figure shows extracellular vesicles can shorten time to occlusion under shear in vitro as compared to plasma (octaplas), which may translate to shortening time to hemostasis in vivo.

FIG. 22 is a graph showing extracellular vesicles (10%) run in Octaplas. Extracellular vesicles Vs promote thrombus formation (occlude microcapillary channel), which indicate hemostatic function and promotion of hemostasis under flow.

FIG. 23 is a graph showing CD9 gating by SSC—H by FSC-H.

FIG. 24 is a graph showing CD9 isotype expression.

FIG. 25 is a graph showing CD9 expression.

FIG. 26 is a graph showing CD41 and CD41 isotype expression.

FIG. 27 is a graph showing CD9 and CD9 isotype expression.

FIG. 28 is a graph showing Lactadherin expression versus unstained samples.

FIG. 29 is a graph showing CD62 and CD62 isotype expression.

FIG. 30 is a graph showing CD42 and CD42 isotype expression.

FIG. 31 is a graph showing 9F9 and 9fF9 isotype expression.

FIG. 32 is a graph showing percent positivity of various cell surface markers. The figure shows high expression of CD41 (platelet-specific marker), CD9 (exosomes marker), lactadherin (phosphatidyl serine expression), and 9F9 (bound fibrinogen), demonstrating the phenotypic characteristics of the final lyophilized extracellular vesicle product.

### DETAILED DESCRIPTION

This disclosure is directed to compositions and methods for use of stabilized platelet-derived extracellular vesicles as biological carriers of cargo, such as, for example, drugs for the therapeutic treatment of hemostasis, wound healing, and various diseases (e.g., cancer). For example, platelets (e.g., any of the platelets described herein) can be loaded with cargo (e.g., cargo further described herein) and subsequently stimulated to generate extracellular vesicles, thus generating loaded extracellular vesicles. The stabilized platelet-derived extracellular vesicles can be derived from fresh, frozen, and/or cold stored platelets and can be subsequently lyophilized (e.g., freeze-dried) either unloaded (e.g., no cargo) or loaded (e.g., loaded with cargo). Stabilized platelet-derived extracellular vesicles can also be derived from platelets without a stimulating agent. For example, platelets can shed extracellular vesicles in the absence of a stimulating agent.

Extracellular vesicles derived from platelets described herein can be stored under typical ambient conditions, refrigerated, cryopreserved, and/or lyophilized after stabilization.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

It is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

As used herein, “thrombosomes” (sometimes also herein called “Tsomes” or “Ts”, particularly in the Examples and Figures) are platelet derivatives that have been treated with an incubating agent (e.g., any of the incubating agents described herein) and lyopreserved (e.g., freeze-dried) to form thrombosomes. In some cases, thrombosomes can be prepared from pooled platelets. Thrombosomes can have a shelf life of 2-3 years in dry form at ambient temperature and can be rehydrated with sterile water within minutes for immediate infusion.

As used herein and in the appended claims, the term “platelet” can include whole platelets, fragmented platelets, platelet derivatives, or thrombosomes. Thus, for example, reference to “cargo-loaded platelets” may be inclusive of drug-loaded platelets as well as drug-loaded platelet derivatives or drug-loaded extracellular vesicles, unless the context clearly dictates a particular form.

As used herein and in the appended claims, the term “fresh platelet” includes platelets stored for less than approximately 24 hours.

As used herein and in the appended claims, the term “stored platelet” includes platelets stored for approximately 24 hours or longer before use.

As used herein and in the appended claims the term “unloaded” includes platelets, platelet derivatives, thrombosomes, and/or extracellular vesicles derived from such platelets that are not loaded with cargo, such as platelets, platelet derivatives, and/or extracellular vesicles that are not loaded with cargo such as a drug.

As used herein, and unless otherwise specified, the terms “treat,” “treating” and “treatment” contemplate an action that occurs while a subject is suffering from a disease (e.g., hemostasis, wound injury, cancer), disorder, and/or condition (e.g., hemorrhage) intended to reduce the severity of the disease, disorder, and/or conditions or slows the progression of the disease, disorder, or condition (“therapeutic treatment”), and which can inhibit the disease, disorder, and/or condition (e.g., hemorrhage).

As used herein a “wound” is an injury to living tissue caused by a cut, blow, and/or other impact to a subject in which the skin (e.g., epidermis, dermis) is damaged (e.g., cut, broken).

In some embodiments, a composition of stabilized platelet-derived extracellular vesicles prepared by any method described herein is for use in regenerative medicine. In some embodiments, a composition of stabilized platelet-derived extracellular vesicles are used in a regenerative medicine method to treat. In some embodiments, regenerative medicine methods can heal or replace tissue damaged by age, disease, and/or trauma. In some embodiments, regenerative medicine methods can normalize congenital defects.

In some embodiments, a regenerative medicine method includes the use of stabilized platelet-derived extracellular vesicles (e.g., any of the extracellular vesicles described herein), where the stabilized platelet-derived extracellular vesicles include one or more growth factors. In some embodiments, a regenerative medicine method includes the use of stabilized platelet-derived extracellular vesicles (e.g., any of the extracellular vesicles described herein), where the stabilized platelet-derived extracellular vesicles include one or more cytokines. In some embodiments, a regenerative medicine method includes the use of stabilized platelet-derived extracellular vesicles (e.g., any of the extracellular vesicles described herein), where the stabilized platelet-derived extracellular vesicles include one or more chemokines.

In some embodiments, rehydrating the cargo-loaded extracellular vesicles comprises adding to the platelets an aqueous liquid. In some embodiments, the aqueous liquid is water. In some embodiments, the aqueous liquid is an aqueous solution. In some embodiments, the aqueous liquid is a saline solution. In some embodiments, the aqueous liquid is a suspension.

As used herein and in the appended claims, the term “platelet derived extracellular vesicle,” “extracellular vesicle,” or “stabilized platelet-derived extracellular vesicles” can include vesicles ranging from about 40 nM to about 450 nM in diameter and such terms can be used interchangeably. Also, as used herein, the aforementioned terms will be understood to include extracellular vesicles derived from platelets (e.g., fresh platelets (e.g., filtered platelets), frozen platelets, lyophilized platelets, cold-stored platelets, platelets already available in the plasma of an apheresis unit, and/or thrombosomes). In some embodiments, an extracellular vesicle is an exosome. In some embodiments, an extracellular vesicle (e.g., an exosome) can be from about 40 nM to about 200 nM in diameter. In some embodiments, an extracellular vesicle is a microvesicle. In some embodiments, an extracellular vesicle (e.g., a microvesicle) can be from about 200 nM to about 450 nM in diameter.

As used herein and described in more detail below, a “stabilizing agent” or a “stabilization agent” refers to an agent included loading buffer to stabilize and thus generate stabilized platelet-derived extracellular vesicles.

As used herein and in the appended claims, “platelet derived” includes extracellular vesicles generated as the result of stimulating platelets (e.g., any of the platelets described herein including thrombosomes) or extra-cellular vesicles derived from non-stimulated platelets (e.g., extracellular vesicles shed from platelets in the absence of a stimulant). Any suitable stimulating agent can be used to stimulate platelets. For example, stimulating agents include aging (e.g., time), chemical agents, and/or mechanical agents.

Extracellular vesicles can be derived from fresh platelets, frozen platelets, and/or cold-stored platelets, and can subsequently be lyophilized. In some embodiments, extracellular vesicles can be derived from loaded platelets, such as for example, platelets loaded with cargo (e.g., a drug). In some embodiments, extracellular vesicles can be derived from previously lyophilized platelets (e.g., thrombosomes).

As used herein, “stabilized” platelet-derived extracellular vesicles (e.g., including extracellular vesicles derived from thrombosomes) are platelet-derived extracellular vesicles that are functional as measured by TGA, T-TAS, and aggregation assays as described herein and further in the Examples. In some embodiments, stabilized platelet-derived extracellular vesicles (e.g., including extracellular vesicles derived from thrombosomes) display platelet-specific expressions markers. In some embodiments, stabilized platelet-derived extracellular vesicles (e.g., including extracellular vesicles derived from thrombosomes), retain loaded cargo (e.g., a drug). In some embodiments, stabilized platelet-derived extracellular vesicles (e.g., including extracellular vesicles derived from thrombosomes), retain a loaded fluorophore.

In some embodiments, platelet-derived extracellular vesicles express platelet-specific surface marker expression, such as for example, CD9, CD61, CD63, 9F9, PAC1, CD42, CD62, Lactadherin, CD142, CD49b, and CD41. In some embodiments, platelet-derived extracellular vesicles express platelet-specific surface markers, such as for example, platelet factor 4 (PF4).

In some embodiments, assaying platelet-specific surface marker expression is performed by a BCA assay. In some embodiments, assaying platelet-specific marker expression is performed with an ELISA kit. In some embodiments, assaying platelet-specific surface marker expression of extracellular vesicles includes comparing lysed extracellular vesicles to unlysed extracellular vesicles.

In some embodiments, platelet-specific surface marker expression is from about 0.1 ng/extracellular vesicles to about 10,000 ng/extracellular vesicle, from about 0.5 ng/extracellular vesicle to about 9,500 ng/extracellular vesicle, from about 1.0 ng/extracellular vesicle to about 9,000 ng/extracellular vesicle, from about 5.0 ng/extracellular vesicle to about 8,500 ng/extracellular vesicle, from about 10.0 ng/extracellular vesicle to about 8,000 ng/extracellular vesicle, from about 50.0 ng/extracellular vesicle to about 7,500 ng/extracellular vesicle, from about 100 ng/extracellular vesicle to about 7,000 ng/extracellular vesicle, from about 200 ng/extracellular vesicle to about 6,500 ng/extracellular vesicle, from about 300 ng/extracellular vesicle to about 6,000 ng/extracellular vesicle, from about 400 ng/extracellular vesicle to about 5,500 ng/extracellular vesicle, from about 500 ng/extracellular vesicle to about 5,000 ng/extracellular vesicle, from about 600 ng/extracellular vesicle to about 4,500 ng/extracellular vesicle, from about 700 ng/extracellular vesicle to about 4,000 ng/extracellular vesicle, from about 800 ng/extracellular vesicle to about 3,500 ng/extracellular vesicle, from about 900 ng/extracellular vesicle to about 3,000 ng/extracellular vesicle, from about 1,000 ng/extracellular vesicle to about 2,500 ng/extracellular vesicle, or from about 1500 ng/extracellular vesicle to about 2,000 ng/extracellular vesicle.

Described herein are compositions and methods for stabilizing platelet derived vesicles (e.g., extracellular vesicles) which can be used for therapeutics applications, such as, for example, hemostasis, wound healing, and cargo (e.g., drug) delivery applications for diseases (e.g., cancer) in addition to, or alternatively, to standard liquid stored platelets.

Thus for example, provided herein is a a method of preparing stabilized platelet-derived extracellular vesicles, the method including generating a population of extracellular vesicles from platelets by allowing the platelets to shed a population of extracellular vesicles over a period of time in the range of approximately 2 days to approximately 21 days, isolating the population of extracellular vesicles and stabilizing the populations of extracellular vesicles with a loading buffer comprising a salt, a base, a loading agent, and optionally at least one organic solvent.

Also provided herein is a a method of preparing stabilized platelet-derived extracellular vesicles, the method including generating a population of extracellular vesicles from platelets over a period of time in the range of approximately 2 days to approximately 21 days, isolating the population of extracellular vesicles, and stabilizing the populations of extracellular vesicles with a loading buffer comprising a salt, a base, a loading agent, and at optionally at least one organic solvent, to form stabilized platelet-derived extracellular vesicles.

Also provided herein a method of preparing stabilized platelet-derived extracellular vesicles, the method including contacting platelets with a stimulating agent to generate a population of extracellular vesicles, isolating the population of extracellular vesicles, and stabilizing the population of extracellular vesicles with a loading buffer comprising a salt, a base, a loading agent, and optionally at least one organic solvent.

Thus for example, also provided herein is a method of preparting stabilized platelet-derived extracellular vesicles, the method including, contacting platelets with a stimulating agent to generate a population of extracellular vesicles, isolating the population of extracellular vesicles and stabilizing the population of extracellular vesicles with a loading buffer comprising a salt, a base, a saccharide, and optionally at least one organic solvent, to form stabilized platelet-derived extracellular vesicles.

In some embodiments of a method of preparing stabilized platelet-derived extracellular vesicles, the stimulating agent includes one of a chemical stimulating agent, a mechanical stimulating agent, or combinations thereof. In some embodiments of a method of preparing stabilized platelet-derived extracellular vesicles, the platelets are contacted with a stimulating agent for a period of time in the range of approximately 5 minutes to approximately 24 hours.

In some embodiments of a method of preparing stabilized platelet-derived extracellular vesicles, the chemical stimulating agent comprises a calcium ionophore, collagen, thrombin, a hypertonic solution, or combinations thereof. In some embodiments of a method of preparing stabilized platelet-derived extracellular vesicles, the mechanical stimulating agent comprises sonication.

In some embodiments of a method of preparing stabilized platelet-derived extracellular vesicles, one or more platelet-derived extracellular vesicles of the population of extracellular vesicles includes an exosome. In some embodiments of a method of preparing stabilized platelet-derived extracellular vesicles, the exosome is in the range of approximately 40 nm to approximately 200 nm in diameter

In some embodiments of a method of preparing stabilized platelet-derived extracellular vesicles, one or more platelet-derived extracellular vesicles includes a microvesicle. In some embodiments of a method of preparing stabilized platelet-derived extracellular vesicles, the microvesicle is in the range of approximately 200 nm to approximately 450 nm.

In some embodiments of a method of preparing stabilized platelet-derived extracellular vesicles, the method includes applying ultracentrifugation to the population of extracellular vesicles. In some embodiments of a method of preparing stabilized platelet-derived extracellular vesicles, the method includes applying filtration to the extracellular vesicles.

In some embodiments of a method of preparing stabilized platelet-derived extracellular vesicles, the loading buffer includes a stabilization agent, where the stabilization agent is a monosaccharide, a disaccharide, a synthetic polymer of a disaccharide, or combinations thereof. In some embodiments of a method of preparing stabilized platelet-derived extracellular vesicles, the loading buffer includes the stabilization agent, where the stabilization agent is sucrose, polysucrose, maltose, trehalose, glucose, mannose, or xylose.

In some embodiments of a method of preparing stabilized platelet-derived extracellular vesicles, the loading buffer includes the stabilization agent, where the stabilization agent includes sucrose and trehalose. In some embodiments of a method of preparing stabilized platelet-derived extracellular vesicles, the loading buffer includes the stabilization agent, where the stabilization agent includes polysucrose and trehalose. In some embodiments of a method of preparing stabilized platelet-derived extracellular vesicles, the trehalose is present in the range of approximately 1% (w/v) to approximately 3% (w/v). In some embodiments of a method of preparing stabilized platelet-derived extracellular vesicles, sucrose is present in the range of approximately 5% (w/v) to approximately 10% (w/v). In some embodiments of a method of preparing stabilized platelet-derived extracellular vesicles, polysucrose is present in the range of approximately 5% (w/v) to approximately 10% (w/v). In some embodiments of a method of preparing stabilized platelet-derived extracellular vesicles, the loading buffer includes PBS and 7% (w/v) sucrose and 2% (w/v) trehalose. In some embodiments of a method of preparing stabilized platelet-derived extracellular vesicles, the loading buffer includes PBS and 7% (w/v) polysucrose and 2% (w/v) trehalose.

In some embodiments of a method of preparing stabilized platelet-derived extracellular vesicles, one or more organic solvents are selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

In some embodiments of a method of preparing stabilized platelet-derived extracellular vesicles, the platelets are isolated prior to the contacting step. In some embodiments of a method of preparing stabilized platelet-derived extracellular vesicles, the platelets are pooled from a plurality of donors prior to the contacting step.

In some embodiments of a method of preparing stabilized platelet-derived extracellular vesicles, the stabilized platelet-derived extracellular vesicles are generated from the group consisting of fresh platelets, stored platelets, frozen platelets, freeze-dried platelets, thrombosomes, and any combination thereof.

In some embodiments of a method of preparing stabilized platelet-derived extracellular vesicles, includes cold storing, cryopreserving, freeze-drying, drying, thawing, rehydrating, or combinations thereof the stabilized platelet-derived extracellular vesicles.

In some embodiments of a method of preparing stabilized platelet-derived extracellular vesicles, the drying step includes freeze-drying the stabilized platelet-derived extracellular vesicles. In some embodiments of a method of preparing stabilized platelet-derived extracellular vesicles, includes rehydrating the stabilized platelet-derived extracellular vesicle.

In some embodiments of a method of preparing stabilized platelet-derived extracellular vesicles, the platelets are further contacted with an imaging agent, where the stabilized platelet derived extracellular vesicle is loaded with the imaging agent.

Also provided herein are stabilized platelet-derived extracellular vesicles prepared by the method of any of the methods described herein.

Thus for example, also provided herein is a composition including stabilized platelet derived extracellular vesicles generated by contacting platelets with a stimulating agent including at least about 50% stabilized platelet derived extracellular vesicles. In some embodiments, the composition including stabilized extracellular vesicles generated by contacting platelets with a stimulating agent including at least about 50% stabilized platelet-derived extraceulluar vesicles by mass are generated by any of the methods described herein.

Also provided herein is a composition including stabilized platelet derived extracellular vesicles generated by contacting platelets with a stimulating agent including at least about 75% stabilized platelet derived extracellular vesicles by mass.

Also provided herein in is a composition including stabilized platelet derived extracellular vesicles generated by contacting platelets with a stimulating agent including at least about 80% stabilized platelet derived extracellular vesicles by mass.

Also provided herein is a composition including stabilized platelet derived extracellular vesicles generated by contacting platelets with a stimulating agent including at least about 90% stabilized platelet derived extracellular vesicles by mass.

Thus for example, also provided herein is a method of treating bleeding in a subject in need thereof, including administering a therapeutically effective amount of stabilized platelet-derived extracellular vesicles generated by contacting platelets with a stimulating agent to the subject in need thereof.

Also provided herein is a method of treating a wound in a subject in need thereof, including administering a therapeutically effective amount of stabilized platelet-derived extracellular vesicles generated by contacting platelets with a stimulating agent to the subject in need thereof.

Also provided herein is a method of treating bleeding in a subject in need thereof, including administering a therapeutically effective amount of the stabilized platelet derived extracellular vesicles generated without a stimulating agent (e.g., aging) to the subject in need thereof.

Also provided herein is a method of treating a wound in a subject in need thereof, including administering a therapeutically effective amount of stabilized platelet-derived extracellular vesicles generated without a stimulating agent (e.g., aging) to the subject in need thereof.

Thus for example, also provided herein are stabilized platelet-derived extracellular vesicles prepared by any of the methods described herein treating or ameliorating regenerative medicine.

Also provided herein is a regenerative medicine method including administering stabilized platelet-derived extracellular vesicles prepared by any method described herein.

Thus for example, provided herein is a regenerative medicine method including adminstering stabilized platelet-derived extracellular vesicles, wherein the stabilized platelet-derived extracellular vesicles comprise one or more growth factors, one or more cytokines, one or more chemokines, and combinations thereof. In some embodiments of a rengenerative medicine method, the one or more growth factors comprises platelet factor 4.

Thus for example, also provided herein is a method of preparing drug-loaded stabilized platelet-derived extracellular vesicles, contacting platelets with a drug and a loading buffer comprising a salt, a base, a loading agent, and optionally at least one organic solvent to form drug-loaded platelets, contacting the drug-loaded platelets with a stimulating agent to generate a population of drug-loaded extracellular vesicles, isolating the population of drug-loaded extracellular vesicles, and stabilizing the population of drug-loaded extracellular vesicles with the loading buffer, to form drug-loaded extracellular vesicles.

Thus for example, also provided herein is a method of preparing drug-loaded stabilized platelet-derived extracellular vesicles, contacting platelets with a drug and a loading buffer comprising a salt, a base, a loading agent, and optionally at least one organic solvent to form drug-loaded platelets, generating a population of drug-loaded extracellular vesicles from the drug-loaded platelets by allowing the drug-loaded platelets to shed the population of extracellular vesicles over a period of time in the range of approximately 2 days to approximately 21 days, isolating the population of drug loaded extracellular vesicles, and stabilizing the population of drug-loaded extracellular vesicles with the loading buffer, to form the drug-loaded extracellular vesicles.

In some embodiments of a method of preparing drug-loaded stabilized platelet-derived extracellular vesicles, the drug and the loading buffer are contacted with the platelets sequentially in either order, or concurrently.

In some embodiments of a method of preparing drug-loaded stabilized platelet-derived extracellular vesicles, the loading buffer includes either sucrose or polysucrose and trehalose. In some embodiments of a method of preparing drug-loaded stabilized platelet-derived extracellular vesicles, the loading buffer comprises trehalose in the range from about 0.1% (w/v) to about 5% (w/v). In some embodiments of a method of preparing drug-loaded stabilized platelet-derived extracellular vesicles, the loading buffer comprises either polysucrose or sucrose in the range from about 2% (w/v) to about 10% (w/v).

In some embodiments of a method of preparing drug-loaded stabilized platelet-derived extracellular vesicles, the drug-loaded stabilized platelet-derived extracellular vesicles treat a disease in a subject in need thereof. In some embodiments of a method of preparing drug-loaded stabilized platelet-derived extracellular vesicles the disease is cancer.

In some embodiments of a method of preparing drug-loaded stabilized platelet-derived extracellular vesicles, includes cold storing, cryopreserving, freeze-drying, thawing, rehydrating, and combinations thereof the drug-loaded stabilized platelet-derived extracellular vesicles.

In some embodiments of a method of preparing drug-loaded stabilized platelet-derived extracellular vesicles, the drug-loaded stabilized platelet-derived extracellular vesicles are generated from the group consisting of drug-loaded fresh platelets, drug-loaded stored platelets, drug-loaded frozen platelets, drug-loaded freeze-dried platelets, drug-loaded thrombosomes, and any combination thereof.

Extracellular vesicles are classified into two main types: microvesicles and exosomes. Exosomes are generally small-sized extracellular vesicles typically in the size range below approximately 200 nm which are formed by the inward budding of multi-vesicular bodies. Extracellular vesicles of this size (e.g., exosomes) generally contain higher concentrations of surface expression markers CD63, CD9, CD81, and tumor susceptibility gene 101 (Tsg 101) relative to microvesicles. Exosome release from platelets is dependent on cytoskeleton activation.

Microvesicles (also known as shedding vesicles) are generally medium to large-sized particles (e.g., approximately over 200 nm in diameter) which are formed by budding from the platelet plasma membrane. Extracellular vesicles of this size (e.g., microvesicles) generally contain higher concentrations of surface expression markers, such as for example, integrins, selectins, and CD40 ligands relative to exosomes. Microvesicle release from platelets is dependent on cytoskeleton activation and calcium influx.

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of extracellular vesicles and/or the dried extracellular vesicles, such as freeze-dried extracellular vesicles, have a particle size in the range of about 30 nm to about 500 nm (e.g., from about 40 nm to about 450 nm, from about 50 nm to about 400 nm, from about 60 nm to about 350 nm, from about 70 nm to about 300 nm, from about 80 nm to about 250 nm, from about 90 nm to about 200 nm, or from about 100 nm to about 150 nm), wherein the percentages above are with respect to the total number of extracellular vesicles.

In some embodiments, at most 99% (e.g., at most about 95%, at most about 80%, at most about 75%, at most about 70%, at most about 65%, at most about 60%, at most about 55%, or at most about 50%) of extracellular vesicles and/or the dried extracellular vesicles, such as freeze-dried (e.g., lyophilized) extracellular vesicles, are in the range of about 30 nm to about 500 nm (e.g., from about 40 nm to about 450 nm, from about 50 nm to about 400 nm, from about 60 nm to about 350 nm, from about 70 nm to about 300 nm, from about 80 nm to about 250 nm, from about 90 nm to about 200 nm, or from about 100 nm to about 150 nm), wherein the percentages above are with respect to the total number of extracellular vesicles.

In some embodiments, about 50% to about 99% (e.g., about 55% to about 95%, about 60% to about 90%, about 65% to about 85, about 70% to about 80%) of extracellular vesicles and/or the dried extracellular vesicles, such as freeze-dried extracellular vesicles (e.g., lyophilized), are in the range of about 30 nm to about 500 nm (e.g., from about 40 nm to about 450 nm, from about 50 nm to about 400 nm, from about 60 nm to about 350 nm, from about 70 nm to about 300 nm, from about 80 nm to about 250 nm, from about 90 nm to about 200 nm, or from about 100 nm to about 150 nm), wherein the percentages above are with respect to the total number of extracellular vesicles.

In some embodiments, a composition comprising stabilized platelet derived extracellular vesicles generated by contacting platelets with a stimulating agent comprises at least about 50% (e.g., at least about 55%, at least about 60%, at least about 65%, at least about 70%, at least about 75%, at least about 80%, at least about 85%, at least about 90%, at least about 95%, or at least about 99%) of extracellular vesicles and/or dried extracellular vesicles, such as freeze-dried extracellular vesicles, by mass, wherein the extracellular vesicles and/or dried extracellular vesicles have a particle size in the range of about 30 nm to about 500 nm (e.g., from about 40 nm to about 450 nm, from about 50 nm to about 400 nm, from about 60 nm to about 350 nm, from about 70 nm to about 300 nm, from about 80 nm to about 250 nm, from about 90 nm to about 200 nm, or from about 100 nm to about 150 nm).

In some embodiments, a composition comprising stabilized platelet derived extracellular vesicles generated by contacting platelets with a stimulating agent comprises at most about 99% (e.g., at most about 95%, at most about 80%, at most about 75%, at most about 70%, at most about 65%, at most about 60%, at most about 55%, or at most about 50%) of extracellular vesicles and/or dried extracellular vesicles, such as freeze-dried (e.g., lyophilized) extracellular vesicles, by mass, wherein the extracellular vesicles and/or dried extracellular vesicles are in the range of about 30 nm to about 500 nm (e.g., from about 40 nm to about 450 nm, from about 50 nm to about 400 nm, from about 60 nm to about 350 nm, from about 70 nm to about 300 nm, from about 80 nm to about 250 nm, from about 90 nm to about 200 nm, or from about 100 nm to about 150 nm) by mass.

In some embodiments, a composition comprising stabilized platelet derived extracellular vesicles generated by contacting platelets with a stimulating agent comprises at least about 50% to about 99% (e.g., about 55% to about 95%, about 60% to about 90%, about 65% to about 85, about 70% to about 80%) of extracellular vesicles and/or dried extracellular vesicles, such as freeze-dried extracellular vesicles (e.g., lyophilized), by mass, wherein the extracellular vesicles and/or dried extracellular vesicles are in the range of about 30 nm to about 500 nm (e.g., from about 40 nm to about 450 nm, from about 50 nm to about 400 nm, from about 60 nm to about 350 nm, from about 70 nm to about 300 nm, from about 80 nm to about 250 nm, from about 90 nm to about 200 nm, or from about 100 nm to about 150 nm) by mass.

In some embodiments, platelets are isolated prior to contacting the platelets with a drug.

In some embodiments, isolating platelets comprises isolating platelets from blood. In some embodiments, platelets are donor-derived platelets. In some embodiments, platelets are donor derived platelets from a single donor. In some embodiments, platelets are donor-derived platelets pooled from two or more donors. In some embodiments, platelets are obtained from whole-blood. In some embodiments, platelets are obtained by a process that comprises an apheresis step.

In some embodiments, platelets, platelet derivatives, or thrombosomes are pooled from a plurality of donors. Such platelets, platelet derivatives, and thrombosomes pooled from a plurality of donors may be also referred herein to as pooled platelets, platelet derivatives, or thrombosomes. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35.

Thus, provided herein in some embodiments is a method of preparing cargo loaded platelets, cargo loaded platelet derivatives, or cargo loaded thrombosomes comprising: step (A) pooling platelets, platelet derivatives, or thrombosomes from a plurality of donors; step (B) contacting the platelets, platelet derivatives, or thrombosomes from step (A) with cargo (e.g., a drug), a cationic transfection reagent, and with a loading buffer comprising a salt, a base, a loading agent, and optionally ethanol, to form the cargo-loaded platelets, the drug-loaded platelet derivatives, or the drug-loaded thrombosomes; and C) stimulating the loaded platelets to generate extracellular vesicles, thus generating loaded extracellular vesicles.

Thus, provided herein in some embodiments is a method of preparing cargo loaded platelets, cargo loaded platelet derivatives, or cargo loaded thrombosomes comprising: step (A) pooling platelets, platelet derivatives, or thrombosomes from a plurality of donors; step (B) contacting the platelets, platelet derivatives, or thrombosomes from step (A) with a cargo (e.g., a drug), a cationic transfection reagent, and with a loading buffer comprising a salt, a base, a loading agent, and optionally ethanol, to form the cargo loaded platelets, the cargo loaded platelet derivatives, or the cargo loaded thrombosomes; and C) collecting extracellular vesicles in the absence of a stimulating agent, thus obtaining loaded extracellular vesicles.

In some embodiments the loading buffer, and/or the liquid medium, may comprise one or more of a) water or a saline solution, b) one or more additional salts, or c) a base. In some embodiments, the loading buffer, and/or the liquid medium, may comprise one or more of a) one or more salts, or b) a base.

In some embodiments, the loading agent is a saccharide. In some embodiments, the saccharide is a monosaccharide. In some embodiments, the saccharide is a disaccharide. In some embodiments, the saccharide is a non-reducing disaccharide. In some embodiments, the disaccharide is a synthetic polymer. For example, polysucrose is a synthetic polymer of sucrose and can be used as a loading agent. In some embodiments, the saccharide is sucrose, maltose, trehalose, glucose (e.g., dextrose), mannose, or xylose. In some embodiments, the loading agent is a starch.

In some embodiments, the loading buffer includes a stabilization agent. In some embodiments, the stabilization agent is a saccharide. In some embodiments, the saccharide is a monosaccharide. In some embodiments, the saccharide is a disaccharide. In some embodiments, the saccharide is a non-reducing disaccharide. In some embodiments, the disaccharide is a synthetic polymer. For example, polysucrose is a synthetic polymer of sucrose and can be used as a stabilization agent. In some embodiments, the saccharide is sucrose, maltose, trehalose, glucose (e.g., dextrose), mannose, or xylose. In some embodiments, the stabilization agent is a starch.

In some embodiments the loading agent is loaded into the platelets in the presence of an aqueous medium. As an example, one embodiment of the methods herein comprises contacting platelets with a drug and with an aqueous loading buffer comprising a salt, a base, a loading agent, and optionally at least one organic solvent, to form the cargo-loaded platelets.

In some embodiments the loading buffer, and/or the liquid medium, may comprise one or more salts selected from phosphate salts, sodium salts, potassium salts, calcium salts, magnesium salts, and any other salt that can be found in blood or blood products, or that is known to be useful in drying platelets, or any combination of two or more of these.

Preferably, these salts are present in the composition at an amount that is about the same as is found in whole blood.

In some embodiments, the drug-loaded platelets are prepared by incubating the platelets with the drug in the liquid medium for different durations at or at different temperatures from 15-45° C., or about 37° C. (cell to drug volume ratio of 1:2).

In some embodiments, the platelets form a suspension in a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

In some embodiments, one or more other components may be loaded in the platelets. In some embodiments, the one or more other components may be loaded concurrently with the cargo (e.g., a drug). In some embodiments, the one or more other components may and the drug may be loaded sequentially in either order.

In some embodiments, a drug loaded into platelets is modified to include an imaging agent. For example, a drug can be modified with an imaging agent in order to image the drug loaded platelet in vivo. In some embodiments, a drug can be modified with two or more imaging agents (e.g., any two or more of the imaging agents described herein). In some embodiments a drug-loaded platelet can be stimulated (e.g., stimulated by any of the stimulating agents described herein) to generate extracellular vesicles. In some embodiments, a drug loaded into platelets is modified with a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as 54Cu, 48V, 52Fe, 55Co, 94Tc or 68Ga; or gamma-emitters such as 171Tc, 111In, 113In, or 67Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to 1231, 131I or 77Br. For example, a positron-emitting radioactive non-metal can include, but is not limited to 11C, 13N, 150, 17F, 18F, 75Br, 76Br or 1241. For example, a hyperpolarized NMR-active nucleus can include, but is not limited to 13C, 15N, 19F, 29Si and 31P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s(dithiolene) complexes, bis(benzene-dithiolate) complexes, iodoaniline dyes, b/stS.0-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals 67Cu, 89Sr, 90Y, 153Sm, 185Re, 188Re or 192Ir, and non-metals 32P, 33P, 38S, 38Cl, 39Cl, 82Br and 83Br. In some embodiments, a drug loaded into platelets can be associated with gold or other equivalent metal particles (such as nanoparticles). For example, a metal particle system can include, but is not limited to gold nanoparticles (e.g., Nanogold™).

In some embodiments, a drug loaded into platelets that is modified with an imaging agent is imaged using an imaging unit. The imaging unit can be configured to image the drug loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Chen Z., et. al., Advance of Molecular Imaging Technology and Targeted Imaging Agent in Imaging and Therapy, Biomed Res Int., February 13, doi: 10.1155/2014/819324 (2014) have described various imaging techniques and which is incorporated by reference herein in its entirety.

In some embodiments, the drug-loaded platelets are prepared by incubating the platelets with the drug in the liquid medium for different durations. The step of incubating the platelets to load one or more cargo, such as a drug(s), includes incubating the platelets for a time suitable for loading, as long as the time, taken in conjunction with the temperature, is sufficient for the drug to come into contact with the platelets and, preferably, be incorporated, at least to some extent, into the platelets. For example, in some embodiments, the drug-loaded platelets are prepared by incubating the platelets with the drug in the liquid medium for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the drug-loaded platelets are prepared by incubating the platelets with the drug in the liquid medium for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the drug-loaded platelets are prepared by incubating the platelets with the drug in the liquid medium from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs.

In some embodiments, the drug-loaded platelets are prepared by incubating the platelets with the drug in the liquid medium at different temperatures. The step of incubating the platelets to load one or more cargo, such as a drug(s), includes incubating the platelets with the drug in the liquid medium at a temperature that, when selected in conjunction with the amount of time allotted for loading, is suitable for loading. In general, the platelets with the drug in the liquid medium are incubated at a suitable temperature (e.g., a temperature above freezing) for at least a sufficient time for the drug to come into contact with the platelets. In embodiments, incubation is conducted at 37° C. In certain embodiments, incubation is performed at 4° C. to 45° C., such as 15° C. to 42° C. For example, in embodiments, incubation is performed at 35° C. to 40° C. (e.g., 37° C.) for 110 to 130 (e.g., 120) minutes and for as long as 24-48 hours.

In some embodiments of a method of preparing drug-loaded platelets disclosed herein, the method further comprises acidifying the platelets, or pooled platelets, to a pH of about 6.0 to about 7.4, prior to a contacting step disclosed herein. In some embodiments, the method comprises acidifying the platelets to a pH of about 6.5 to about 6.9. In some embodiments, the method comprises acidifying the platelets to a pH of about 6.6 to about 6.8. In some embodiments, the acidifying comprises adding to the pooled platelets a solution comprising Acid Citrate Dextrose.

In some embodiments, the platelets are isolated prior to a contacting step. In some embodiments, the method further comprises isolating platelets by using centrifugation. In some embodiments, the centrifugation occurs at a relative centrifugal force (RCF) of about 800 g to about 2000 g. In some embodiments, the centrifugation occurs at relative centrifugal force (RCF) of about 1300 g to about 1800 g. In some embodiments, the centrifugation occurs at relative centrifugal force (RCF) of about 1500 g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

In some embodiments, the drug can be a drug for the treatment of cancer. In some embodiments, the drug can be a nucleic acid, a protein, a small molecule (e.g., an organic compound having a molecular weight up to about 2 kDalton), an anti-fibrinolytic, an oligopeptide, and/or an aptamer. In some embodiments, the drug can be RNA, such as for example, mRNA, rRNA, tRNA, snoRNA, hairpin RNA, miRNA, or siRNA, excluding miRNA23a and miRNA27a. For example, the drug can be any of the drugs included in U.S. patent application Ser. No. 16/697,401, U.S. patent application Ser. No. 16/698,645, and U.S. patent application Ser. No. 16/697,607, each of which is incorporated herein by reference in their entireties)

In various methods described herein, platelets are loaded with one or more any of a variety of drugs. In some embodiments, platelets are loaded with a small molecule. For example, platelets can be loaded with one or more of vemurafenib (ZELBORAF®), dabrafenib (TAFINLAR®), encorafenib (BRAFTOVI™), BMS-908662 (XL281), sorafenib, LGX818, PLX3603, RAF265, R05185426, GSK2118436, ARQ 736, GDC-0879, PLX-4720, AZ304, PLX-8394, HM95573, RO5126766, LXH254, trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, CS3006, WX-554, PD98059, CI1040 (PD184352), hypothemycin, FRI-20 (ON-01060), VTX-11e, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-oxozeaenol, 5-iodotubercidin, GDC0994, ONC201, buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, GSK2636771, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, erucylphophocholine, erufosine, SR13668, OSU-A9, PH-316, PHT-427, PIT-1, DM-PIT-1, triciribine (triciribine phosphate monohydrate), API-1, N-(4-(5-(3-acetamidophenyl)-2-(2-aminopyridin-3-yl)-3H-imidazo[4,5-b]pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, MLN0128, AZD-2014, CC-223, AZD2014, CC-115, everolimus (RAD001), temsirolimus (CCI-779), ridaforolimus (AP-23573), tipifarnib, BMS-214662, L778123, L744832, FTI-277, PRI-724, CWP232291, PNU74654, PKF115-584, PKF118-744, PKF118-310, PFK222-815, CGP 049090, ZTM000990, BC21, methyl 3-{[(4-methylphenyl)sulfonyl]amino}benzoate (MSAB), AV65, iCRT3, iCRT5, iCRT14, SM04554, LGK 974, XAV939, curcumin (e.g., Meriva®), DIF-1, genistein, NSC668036, FJ9, BML-286 (3289-8625), IWP, IWP-1, IWP-2, JW55, G007-LK, pyrvinium, foxy-5, Wnt-5a, ipafricept (OMP-54F28), vantictumab (OMP-18R5), SM04690, SM04755, nutlin-3a, IWR1, JW74, okadaic acid, SB239063, SB203580, adenosine diphosphate (hydroxymethyl)pyrrolidinediol (ADP-HPD), 2-[4-(4-fluorophenyl)piperazin-1-yl]-6-methylpyrimidin-4(3H)-one, PJ34, J01-017a, IC261, PF670462, bosutinib (BOSULIF®), PHA665752, imatinib (GLEEVEC®), ICG-001, Rp-8-Br-cAMP, SDX-308, WNT974, CGX1321, ETC-1922159, AD-REIC/Dkk3, WIKI4, windorphen, NTRC 0066-0, CFI-402257, a (5,6-dihydro)pyrimido[4,5-e]indolizine, BOS172722, S63845, AZD5991, AMG 176, 483-LM, MIK665, TASIN-1 (Truncated APC Selective Inhibitor), osimertinib (AZD9291, merelectinib, TAGRISSO™), erlotinib (TARCEVA®), gefitinib (IRESSA®), neratinib (HKI-272, NERLYNX®), lapatinib (TYKERB®), vetanib (CAPRELSA®), rociletinib (CO-1686), olmutinib (OLITA™, HM61713, BI-1482694), naquotinib (ASP8273), nazartinib (EGF816, NVS-816), PF-06747775, icotinib (BPI-2009H), afatinib (BIBW 2992, GILOTRIF®), dacomitinib (PF-00299804, PF-804, PF-299, PF-299804), avitinib (AC0010), ACOO1OMA EAI045, canertinib (CI-1033), poziotinib (NOV120101, HM781-36B), AV-412, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, GW572016, pyrotinib (SHR1258), palbociclib, ribociclib, abemaciclib, olaparib, veliparib, iniparib, rucaparib, CEP-9722, E7016, E7449, PRN1371, BLU9931, FIIN-4, H3B-6527, NVP-BGJ398, ARQ087, TAS-120, CH5183284, Debio 1347, INCB054828, JNJ-42756493 (erdafitinib), rogaratinib (BAY1163877), FIIN-2, LY2874455, lenvatinib (E7080), ponatinib (AP24534), regorafenib (BAY 73-4506), dovitinib (TKI258), lucitanib (E3810), cediranib (AZD2171), nintedanib (OFEV®, BIBF 1120), brivanib (BMS-540215), ASP5878, AZD4547, BGJ398 (infigratinib), E7090, HMPL-453, MAX-40279, XL999, orantinib (SU6668), pazopanib (VOTRIENT®), anlotinib, AL3818, PRIMA-1 (p53 reactivation induction of massive apoptosis-1), APR-246 (PRIMA-1MET), 2-sulfonylpyrimidines such as PK11007, pyrazoles such as PK7088, zinc metallochaperone-1 (ZMC1; NSC319726/ZMC 1), a thiosemicarbazone (e.g., COTI-2), CP-31398, STIMA-1 (SH Group-Targeting Compound That Induces Massive Apoptosis), MIRA-1 (NSC19630) and its analogs, e.g., MIRA-2 and MIRA-3, RITA (NSC652287), chetomin (CTM), stictic acid (NSC87511), p53R3, SCH529074, WR-1065, arsenic compounds, gambogic acid, spautin-1, YK-3-237, NSC59984, disulfiram (DSF), G418, RETRA (reactivate transcriptional activity), PD0166285, 17-AAG, geldanamycin, ganetespib, AUY922, IPI-504, vorinostat/SAHA, romidepsin/depsipeptide, HBI-8000, RG7112 (R05045337), R05503781, MI-773 (SAR405838), DS-3032b, AM-8553, AMG 232, MI-219, MI-713, MI-888, TDP521252, NSC279287, PXN822, ATSP-7041, spiroligomer, PK083, PK5174, PK5196, nutlin 3a, RG7388, Ro-2443, FTY-720, ceramide, OP449, vatalanib (PTK787/ZK222584), TKI-538, sunitinib (SU11248, SUTENT®), thalidomide, lenalidomide (REVLIMID®), axitinib (AG013736, INLYTA®), RXC0004, ETC-159, LGK974, WNT-C59, AZD8931, AST1306, CP724714, CUDC101, TAK285, AC480, DXL-702, E-75, PX-104.1, ZW25, CP-724714, irbinitinib (ARRY-380, ONT-380), TAS0728, AST-1306, AEE-788, perlitinib (EKB-569), PKI-166, D-69491, HKI-357, AC-480 (BMS-599626), RB-200h, ARRY-334543 (ARRY-543, ASLAN001), CUDC-101, IDM-1, decitabine, cytosine arabinoside, ORY1001 (RG6016), GSK2879552, INCB059872, IMG7289, CC90011, MI1, MI2, MI3, Mi2-2 (MI-2-2), MI463, MI503, MIV-6R, EPZ004777, EPZ-5676, SGC0946, CN-SAH, SYC-522, SAH, SYC-534, MM-101, MM-102, MM-103, MM-401, WDR5-0101, WDR5-0102, WDR5-0103, OICR-9429, tivantinib (ARQ 197), golvatinib (E7050), cabozantinib (XL 184, BMS-907351), foretinib (GSK1363089), crizotinib (PF-02341066), MK-2461, BPI-9016M, TQ-B3139, MGCD265, MK-8033, capmatinib (INC280, INCB28060), tepotinib (MSC2156119J, EMD1214063), CE-35562, AMG-337, AMG-458, PHA-665725, PF-04217903, SU11274, PHA-665752, HS-10241, ARGX-111, glumetinib (SCC244), EMD 1204831, AZD6094 (savolitinib, volitinib, HMPL-504), PLB1001, ABT-700, AMG 208, INCB028060, AL2846, HTI-1066, PT2385, PT2977, 17 allylamino-17-demethoxygeldanamycin, eribulin (HALAVEN®, E389, ER-086526), ibrutinib (PCI-32675, Imbruvica®) (1-[(3R)-3-[4-amino-3-(4-phenoxyphenyl)pyrazolo[3,4-d]pyrimidin-1-yl]piperidin-1-yl]prop-2-en-1-one); ACO0058 (ACO0058TA); N-(3-((2-((3-fluoro-4-(4-methylpiperazin-1-yl)phenyl)amino)-7H-pyrrolo[2,3-d]pyrimidin-4-yl)oxy)phenyl)acrylamide; acalabrutinib (ACP-196, Calquence®, rINN) (4-[8-amino-3-[(2S)-1-but-2-ynoylpyrrolidin-2-yl]imidazo[1,5-a]pyrazin-1-yl]-N-pyridin-2-ylbenzamide); zanubrutinib (BGB-3111) ((7R)-2-(4-phenoxyphenyl)-7-(1-prop-2-enoylpiperidin-4-yl)-1,5,6,7-tetrahydropyrazolo[1,5-a]pyrimidine-3-carboxamide); spebrutinib (AVL-292, 1202757-89-8, Cc-292) (N-[3-[[5-fluoro-2-[4-(2-methoxyethoxy)anilino]pyrimidin-4-yl]amino]phenyl]prop-2-enamide); poseltinib (HM71224, LY3337641) (N-[3-[2-[4-(4-methylpiperazin-1-yl)anilino]furo[3,2-d]pyrimidin-4-yl]oxyphenyl]prop-2-enamide); evobrutinib (MSC 2364447, M-2951) (1-[4-[[[6-amino-5-(4-phenoxyphenyl)pyrimidin-4-yl]amino]methyl]piperidin-1-yl]prop-2-en-1-one); tirabrutinib (ONO-4059, GS-4059, ONO/GS-4059, ONO-WG-307) (1-[4-[[[6-amino-5-(4-phenoxyphenyl)pyrimidin-4-yl]amino]methyl]piperidin-1-yl]prop-2-en-1-one); vecabrutinib (SNS-062) ((3R,4S)-1-(6-amino-5-fluoropyrimidin-4-yl)-3-[(3R)-3-[3-chloro-5-(trifluoromethyl)anilino]-2-oxopiperidin-1-yl]piperidine-4-carboxamide); dasatinib (Sprycel®; BMS-354825) (N-(2-chloro-6-methylphenyl)-2-[[6-[4-(2-hydroxyethyl)piperazin-1-yl]-2-methylpyrimidin-4-yl]amino]-1,3-thiazole-5-carboxamide); PRN1008, PRN473, ABBV-105, CG′806, ARQ 531, BIIB068, AS871, CB1763, CB988, GDC-0853, RN486, GNE-504, GNE-309, BTK Max, CT-1530, CGI-1746, CGI-560, LFM A13, TP-0158, dtrmwxhs-12, CNX-774, entrectinib, nilotinib, 1-((3S,4R)-4-(3-fluorophenyl)-1-(2-methoxyethyl)pyrrolidin-3-yl)-3-(4-methyl-3-(2-methylpyrimidin-5-yl)-1-phenyl-1H-pyrazol-5-yl)urea, AG 879, AR-772, AR-786, AR-256, AR-618, AZ-23, AZ623, DS-6051, Go 6976, GNF-5837, GTx-186, GW 441756, LOXO-101, LOXO-195, MGCD516, PLX7486, RXDX101, TPX-0005, TSR-011, venetoclax (ABT-199, RG7601, GDC-0199), navitoclax (ABT-263), ABT-737, TW-37, sabutoclax, obatoclax, BIX-01294 (BIX), UNC0638, A-366, UNC0642, DCG066, UNC0321, BRD 4770, UNC 0224, UNC 0646, UNC0631, BIX-01338, INNO-406, KX2-391, saracatinib, PP1, PP2, ruxolitinib, lestaurtinib (CEP-701), momelotinib (GS-0387, CYT-387), pacritinib (SB1518), fedratinib (SAR302503), BI2536, BI6727, GSK461364, amsacrine, azacitidine, busulfan, carboplatin, capecitabine, chlorambucil, cisplatin, cyclophosphamide, cytarabine, dacarbazine, daunorubicin, docetaxel, doxifluridine, doxorubicin, epirubicin, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, streptozocin, tafluposide, temozolomide, teniposide, tioguanine, topotecan, uramustine, valrubicin, vinblastine, vincristine, vindesine, and vinorelbine.

In various methods described herein, platelets are loaded with one or more any of a variety of drugs. In some embodiments, platelets are loaded with a protein (e.g., an antibody or antibody conjugate). For example, platelets can be loaded with one or more of cetuximab (ERBITUX®), necitumumab (PORTRAZZA™, IMC-11F8), panitumumab (ABX-EGF, VECTIBIX®), matuzumab (EMD-7200), nimotuzumab (h-R3, BIOMAb EGFR®), zalutumab, MDX447, OTSA 101, OTSA101-DTPA-90Y, ABBV-399, depatuxizumab (humanized mAb 806, ABT-806), depatuxizumab mafodotin (ABT-414), SAIT301, Sym004, MAb-425, Modotuximab (TAB-H49), futuximab (992 DS), zalutumumab, Sym013, AMG 595, JNJ-61186372, LY3164530, IMGN289, KL-140, R05083945, SCT200, CPGJ602, GP369, BAY1187982, FPA144 (bemarituzumab), bevacizumab (AVASTIN®), ranibizumab, trastuzumab (HERCEPTIN®), pertuzumab (PERJETA®), trastuzumab-dkst (OGIVRI®), ado-trastuzumab emtansine (KADCYLA®, T-DM1), Zemab, DS-8201a, MFGR1877S, B-701, rilotumumab (AMG102), ficlatuzumab (AV-299), FP-1039 (GSK230), TAK701, YYB101, onartuzumab (MetMAb), ipilimumab (YERVOY®), tremelimumab (CP-675,206), pembrolizumab (KEYTRUDA®), nivolumab (OPDIVO®), atezolizumab (TECENTRIQ®), avelumab (BAVENCIO®), durvalumab (IMFINZI™), BIOO-1, BIOO-2, and BIOO-3.

In various methods described herein, platelets are loaded with one or more any of a variety of drugs. In some embodiments, platelets are loaded with an oligopeptide. For example, platelets can be loaded with one or more of RGD-SSL-Dox, LPD-PEG-NGR, PNC-2, PNC-7, RGD-PEG-Suc-PD0325901, VWCS, FWCS, p16, Bac-7-ELP-p21, Pen-ELP-p21, TAT-Bim, Poropeptide-Bax, R8-Bax, CT20p-NP, RRM-MV, RRM-IL12, PNC-27, PNC-21, PNC-28, Tat-aHDM2, Int-H1-S6A, F8A, Pen-ELP-H1, BACl-ELP-H1, goserelin, leuprolide, Buserelin, Triptorelin, Degarelix, Pituitary adenylate cyclase activating peptide (PACAP), cilengitide, ATN-161 (AcPHSCN-NH2), TTK, LY6K, IMP-3, P16_37-63, VEGFR1-A24-1084, uMMP-2, uTIMP-1, MIC-1/GDF15, RGS6, LGR5, PGI/II, CA242, EN2, UCP2, a HER-2 peptide, MUCIm, HNP1-3, L-glutamine L-tryptophan (IM862), CPAA-783-EPPT1, serum C-peptide, WT1, KIF20A, GV1001, LY6K-177, PAP-114-128, E75, SU18, SU22, ANP, TCP-1, F56, WT1, TERT572Y, disruptin, TREM-1, LFC131, BPP, TH10, BC71, RC-3095, RC-3940-II, RC-3950, (KLAKLAK)2, RGD-(KLAKLAK)2, NGR-(KLAKLAK)2, and SAH-8 (stapled peptides).

In some embodiments, the platelets are at a concentration from about 2,000 platelets/μl to about 500,000,000 platelets/μl. In some embodiments, the platelets are at a concentration from about 50,000 platelets/μl to about 4,000,000 platelets/μl. In some embodiments, the platelets are at a concentration from about 100,000 platelets/μl to about 300,000,000 platelets/μl. In some embodiments, the platelets are at a concentration from about 1,000,000 to about 2,000,000. In some embodiments, the platelets are at a concentration of about 200,000,000 platelets/μl.

In some embodiments, the buffer is a loading buffer comprising the components as listed in Table 1 herein. In some embodiments, the loading buffer comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the loading buffer includes from about 0.5 mM to about 100 mM of the one or more salts. In some embodiments, the loading buffer includes from about 1 mM to about 100 mM (e.g., about 2 mM to about 90 mM, about 2 mM to about 6 mM, about 50 mM to about 100 mM, about 60 mM to about 90 mM, about 70 to about 85 mM) about of the one or more salts. In some embodiments, the loading buffer includes about 5 mM, about 75 mM, or about 80 mM of the one or more salts.

In some embodiments, the loading buffer includes one or more buffers, e.g., N-2-hydroxyethylpiperazine-N′-2-ethanesulfonic acid (HEPES), or sodium-bicarbonate (NaHCO₃). In some embodiments, the loading buffer includes from about 5 to about 100 mM of the one or more buffers. In some embodiments, the loading buffer includes from about 5 to about 50 mM (e.g., from about 5 mM to about 40 mM, from about 8 mM to about 30 mM, about 10 mM to about 25 mM) about of the one or more buffers. In some embodiments, the loading buffer includes about 10 mM, about 20 mM, about 25 mM, or about 30 mM of the one or more buffers.

In some embodiments, the loading buffer includes one or more saccharides, such as monosaccharides and disaccharides, including sucrose, maltose, trehalose, glucose, mannose, dextrose, and xylose. In some embodiments, the loading buffer includes from about 10 mM to about 1,000 mM of the one or more saccharides. In some embodiments, the loading buffer includes from about 50 to about 500 mM of the one or more saccharides. In embodiments, one or more saccharides is present in an amount of from 10 mM 10 to 500 mM. In some embodiments, one or more saccharides is present in an amount of from 50 mM to 200 mM. In embodiments, one or more saccharides is present in an amount from 100 mM to 150 mM.

In some embodiments, the loading buffer includes adding an organic solvent, such as ethanol, to the loading solution. In such a loading buffer, the solvent can range from about 0.1% (v/v) to about 5.0% (v/v), such as from about 0.3% (v/v) to about 3.0% (v/v), or from about 0.5% (v/v) to about 2% (v/v).

In some embodiments, after isolating platelets as described herein, the platelets (e.g., any platelet described herein) are stimulated by a stimulating agent. In some embodiments, a stimulating agent is an agent capable of, for example, activating platelets such that extracellular vesicles (e.g., exosomes and microvesicles) are generated from the platelets, thus generating platelet-derived extracellular vesicles.

In some embodiments, the platelets are contacted with the stimulating agent for a period of time from about 5 minutes to about 24 hours. In some embodiments, the platelets are contacted with the stimulating agent for a period of time from about 30 minutes to about 16 hours. In some embodiments, the platelets are contacted with the stimulating agent for a period of time from about 1 hour to about 12 hours. In some embodiments, the platelets are contacted with the stimulating agent for a period of time from about 2 hours to about 10 hours. In some embodiments, the platelets are contacted with the stimulating agent for a period of time from about 3 hours to about 9 hours. In some embodiments, the platelets are contacted with the stimulating agent for a period of time from about 4 hours to about 8 hours. In some embodiments, the platelets are contacted with the stimulating agent for a period of time from about 5 hours to about 7 hours.

In some embodiments, the platelets are contacted with the stimulating agent for a period of time from about 5 minutes to about 60 minutes. In some embodiments, the platelets are contacted with the stimulating agent for a period of time from about 10 minutes to about 55 minutes. In some embodiments, the platelets are contacted with the stimulating agent for a period of time from about 15 minutes to about 45 minutes. In some embodiments, the platelets are contacted with the stimulating agent for a period of time from about 20 minutes to about 40 minutes. In some embodiments, the platelets are contacted with the stimulating agent for a period of time from about 25 minutes to about 35 minutes. In some embodiments, the platelets are contacted with the stimulating agent for a period of time of about 30 minutes.

As used herein, “shed” or “shedding” refers to generating extracellular vesicles (e.g., microvesicles and exosomes) from platelets (e.g., any of the platelets described herein), fragmented platelets, platelet derivatives, or thrombosomes. In some embodiments, platelets naturally shed extracellular vesicles over a period of time such as days, such as for example, a period of about 1 to about 21 days, a period of about 2 to 20 days, a period of about 3 to about 19 days, a period of about 4 to about 18 days, a period of about 5 to about 17 days, a period of about 6 to about 16 days, a period of about 7 to about 15 days, a period of about 8 to about 14 days, a period of about 9 to about 13 days, a period of about 10 to about 12 days, a period of about 11 days, or a period of about 5 days. For example, platelets can shed extracellular vesicles over time over a period of days in the absence of a stimulating agent. In some embodiments, platelets, fragmented platelets, platelet derivatives, or thrombosomes are provided and allowed to shed a population of extracellular vesicles for a period of time, such as for a period of about 2 days to about 21 days.

In some embodiments, platelets shed extracellular vesicles over a range of temperatures, such as for example, a temperature of about 3° C. to about 20° C., a temperature of about 4° C. to about 19° C., a temperature of about 5° C. to about 18° C., a temperature of about 6° C. to about 17° C., a temperature of about 7° C. to about 16° C., a temperature of about 8° C. to about 15° C., a temperature of about 9° C. to about 14° C., a temperature of about 10° C. to about 13° C., a temperature of about 11° C. to about 12° C.

In some embodiments, platelets shed extracellular vesicles over 14 days at 4° C.

In some embodiments, the stimulating agent is a chemical agent. Non-limiting examples of a chemical stimulating agent include a calcium ionophore (e.g., calcium ionophores known in the art), collagen, thrombin, a hypertonic solution, or combinations thereof.

In some embodiments, the stimulating agent is a calcium ionophore. Any suitable calcium ionophore known in the art can be used as a stimulating agent. Non-limiting suitable examples of calcium ionophores include A23187 (Calcimycin) and Iononycin.

In some embodiments, the platelets are loaded by a process comprising osmotic hypertonic/hypotonic loading/hypotonic shock (U.S. patent application Ser. No. 16/698,583, which is incorporated herein by reference in its entirety). Hypotonic shock uses a solution with lower osmotic pressure to induce cell swelling leading to membrane permeability. Hypertonic shock may increase platelet loading of cryoprotectants or lyoprotectants (e.g., trehalose) (Zhou X., et. al., Loading Trehalose into Red Blood Cells by Improved Hypotonic Method, Cell Preservation Technology, 6(2), https://doi.org/10.1089/cpt.2008.0001 (2008) which is herein incorporated by reference). Additionally and alternatively, hypotonic shock may allow the uptake and internalization of large and/or charged molecules through passive means, such as, endocytosis, micropinocytosis, and/or diffusion.

In some embodiments, hypertonic/hypotonic loading comprises an osmotic gradient to drive pore formation in the platelet's cell membrane and influx cargo intracellularly. In some embodiments, platelets may be isolated (e.g., centrifuged) and resuspended in a hypertonic pre-treatment solution. In some embodiments, the pre-treatment solution can be a carbohydrate in a buffer. For example, the carbohydrate can be a monosaccharide. In a non-limiting way, suitable monosaccharides include: fructose (levulose), galactose, ribose, deoxyribose, xylose, mannose, and fucose. In some embodiments, the carbohydrate can be a disaccharide. In a non-limiting way, suitable disaccharides include: sucrose, lactose, maltose, lactulose, trehalose, and cellobiose. In some embodiments, the carbohydrate can be dextrose in PBS buffer. In some embodiments, the percent dextrose can be about 15% dextrose in PBS to about 20% dextrose in PBS. In some embodiments, the percent dextrose in PBS can be about 16%, about 17%, about 18%, and about 19% dextrose in PBS. In some embodiments, the platelets can be pre-treated for about 10 minutes to about 60 minutes. In some embodiments, the platelets can be pre-treated for about 20, about 30, about 40, and about 50 minutes.

In some embodiments, the solutes in the hypertonic solution can be, in a non-limiting way, salts, low-molecular weight sugars (e.g., monosaccharides, disaccharides), or low molecular weight inert hydrophilic polymers.

In some embodiments, hypertonic/hypotonic loading is used to load water soluble cargo.

In some embodiments, the cargo is at a concentration of about 1 mM to about 1 M, about 10 mM to about 900 mM, about 20 mM to about 800 mM, about 30 mM to about 700 mM, about 40 mM to about 600 mM, about 50 mM to about 500 mM, about 60 mM to about 400 mM, about 70 mM to about 300 mM, about 80 mM to about 200 mM, or about 90 mM to about 100 mM. In some embodiments the cargo is at a concentration of about 500 mM to about 10 M, about 600 mM to about 9 M, about 700 mM to about 8 M, about 800 mM to about 7 M, about 900 mM to about 6 M, about 1 M to about 5 M, about 2 M to about 4 M, or about 3 M.

In some embodiments, the stimulating agent is a mechanical agent. For example, the mechanical agent is sonication. In some embodiments, the mechanical agent is a shear force. In some embodiments, the mechanical agent is electroporation. In some embodiments, the mechanical agent is a freeze-thaw process.

In some embodiments, after stimulating the platelets and generating platelet-derived extracellular vesicles, the stabilized platelet-derived extracellular vesicles are isolated from platelets, fragmented platelets, platelet-derivatives, or thrombosomes. As used herein, “isolating” or “isolated” stabilized platelet-derived extracellular vesicles include a composition substantially free of platelets, fragmented platelets, platelet derivatives, or thrombosomes. In some embodiments, isolated stabilized platelet-derived extracellular vesicles include a composition at least 50% free of platelets, fragmented platelets, platelet derivatives, or thrombosomes, a composition at least 55% free of platelets, fragmented platelets, platelet derivatives, or thrombosomes, a composition at least 60% free of platelets, fragmented platelets, platelet derivatives, or thrombosomes, a composition at least 65% free of platelets, fragmented platelets, platelet derivatives, or thrombosomes, a composition at least 70% free of platelets, fragmented platelets, platelet derivatives, or thrombosomes, a composition at least 75% free of platelets, fragmented platelets, platelet derivatives, or thrombosomes, a composition at least 80% free of platelets, fragmented platelets, platelet derivatives, or thrombosomes, a composition at least 85% free of platelets, fragmented platelets, platelet derivatives, or thrombosomes, a composition at least 90% free of platelets, fragmented platelets, platelet derivatives, or thrombosomes, a composition at least 95% free of platelets, fragmented platelets, platelet derivatives, or thrombosomes, or a composition at least 96%, at least 97%, at least 98%, or at least 99% free of platelets, fragmented platelets, platelet derivatives, or thrombosomes. In some embodiments, isolating stabilized platelet-derived extracellular vesicles are isolated from non-stimulated platelets.

In some embodiments, after stimulating the platelets and generating platelet-derived extracellular vesicles, the stabilized platelet-derived extracellular vesicles are isolated from platelets, fragmented platelets, platelet-derivatives, or thrombosomes. In some embodiments, isolating stabilized platelet-derived extracellular vesicles include a composition substantially comprising stabilized platelet-derived extracellular vesicles, such as for example, a composition including at least about 50% stabilized platelet-derived extracellular vesicles, at least about 55% stabilized platelet-derived extracellular vesicles, at least about 60% stabilized platelet-derived extracellular vesicles, at least about 65% stabilized platelet-derived extracellular vesicles, at least about 70% stabilized platelet-derived extracellular vesicles, at least about 75% stabilized platelet-derived extracellular vesicles, at least about 80% stabilized platelet-derived extracellular vesicles, at least about 85% stabilized platelet-derived extracellular vesicles, at least about 90% stabilized platelet-derived extracellular vesicles, at least about 95% stabilized platelet-derived extracellular vesicles, at least about 96%, 97%, 98%, or 99% stabilized platelet-derived extracellular vesicles.

In some embodiments, after stimulating the platelets and generating platelet-derived extracellular vesicles, the stabilized platelet-derivative extracellular vesicles can be stabilized in a loading buffer. For example, the loading buffer can be the loading buffer described in Table 1 (e.g., loading buffer 1). In some embodiments, the loading buffer is PBS buffer (about pH 6.6 to about pH 6.8) including either sucrose or polysucrose and trehalose (e.g., loading buffer 2). In some embodiments, the loading buffer stabilizes the platelet-derived extracellular vesicles.

In some embodiments, the loading buffer is PBS buffer from about pH 6.2 to about pH 7.2. In some embodiments, the loading buffer is PBS buffer at about pH 6.3, 6.4, 6.5, 6.6. 6.7, 6.8, 6.9, 7.0, 7.1, or 7.2.

In some embodiments, the PBS loading buffer includes a disaccharide. In some embodiments the disaccharide is trehalose. In some embodiments, the trehalose is from about 0.1% to about 10% (w/v), from about 0.25% to about 9%, from about 0.5% to about 8%, from about 0.75% to about 7%, from about 1% to about 6%, from about 1.25% to about 5%, from about 1.5% to about 4%, and from about 1.75% to about 3%. In some embodiments, the trehalose is about 2% (w/v).

In some embodiments, the disaccharide is sucrose. In some embodiments, the sucrose is from about 1% to about 12% (w/v), from about 2% to about 11%, from about 3% to about 10%, from about 4% to about 9%, from about 5% to about 8%, from about 6% to about 7%. In some embodiments, the sucrose is about 7% (w/v).

In some embodiments, the PBS loading buffer includes polysucrose instead of sucrose. In some embodiments, the polysucrose is from about 1% to about 12% (w/v), from about 2% to about 11%, from about 3% to about 10%, from about 4% to about 9%, from about 5% to about 8%, from about 6% to about 7%. In some embodiments, the polysucrose is about 7% (w/v).

In some embodiments, the loading buffer (e.g., loading buffer 2) is PBS buffer (pH from about 6.6 to about 6.8), trehalose at 2% (w/v), and either sucrose or polysucrose at 7% (w/v) in buffer. As used herein, “w/v” in reference to a concentration of a component in a buffer refers to the weight of the component divided by the volume of the buffer.

In some embodiments, stabilized platelet-derived extracellular vesicles (e.g., stabilized in loading buffer 1 or loading buffer 2) are isolated from the platelets.

In some embodiments, the stabilized platelet-derived extracellular vesicles are derived by ultracentrifugation. In some embodiments, ultracentrifugation is performed from about 90,000 g to about 160,000 g. In some embodiments, ultracentrifugation is performed from about 95,000 g to about 155,000 g. In some embodiments, ultracentrifugation is performed from about 100,000 g to about 150,000 g. In some embodiments, ultracentrifugation is performed from about 105,000 g to about 145,000 g. In some embodiments, ultracentrifugation is performed from about 110,000 g to about 140,000 g. In some embodiments, ultracentrifugation is performed from about 115,000 g to about 135,000 g. In some embodiments, ultracentrifugation is performed from about 120,000 g to about 130,000 g. In some embodiments, ultracentrifugation is performed at about 125,000 g.

In some embodiments, stabilized platelet-derived extracellular vesicles are derived by ultracentrifugation performed from about 2° C. to about 10°, from about 3° C. to about 9° C., from about 4° C. to about 8° C., from about 5° to about 7° C., from about 6° C. In some embodiments, stabilized platelet-derived extracellular vesicles are isolated at about 4° C.

In some embodiment, stabilized platelet-derived extracellular vesicles are derived by ultracentrifugation performed from about 30 minutes to about 120 minutes, from about 35 minutes to about 115 minutes, from about 30 minutes to about 110 minutes, from about 40 minutes to about 105 minutes, from about 45 minutes to about 100 minutes, from about 50 minutes to about 95 minutes, from about 55 minutes to about 90 minutes, from about 60 minutes to about 85 minutes, from about 60 minutes to about 85 minutes, from about 65 minutes to about 80 minutes, from about 70 minutes to about 75 minutes. In some embodiments, stabilized platelet-derived extracellular vesicles are derived by ultracentrifugation performed for about 60 minutes.

In some embodiments, stabilized platelet-derived extracellular vesicles are derived by ultracentrifugation performed at about 100,000 g for about 60 minutes at 4° C.

In some embodiments, the stabilized platelet-derived extracellular vesicles are isolated by filtration. Any suitable filtration method can be used to selectively separate platelet-derived extracellular vesicles based on size (e.g., by sizes described herein). In some embodiments, filtration is tangential flow filtration (TFF). In some embodiments, filtration is performed with a syringe filter. In some embodiments, platelet-derived extracellular vesicles can be isolated by filtration with a 0.45 μm syringe filter (e.g., isolating platelet-derived extracellular vesicles 0.45 μm or smaller).

In some embodiments, after stabilizing extracellular vesicles generated from platelets (e.g., any of the platelets described herein), the extracellular vesicles are freeze-dried (e.g., lyophilized). In some embodiments, extracellular vesicles are derived from freeze-dried platelets (e.g., thrombosomes).

Any known technique for drying extracellular vesicles can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in below in Protocol 1: Lyophilization Protocol. Additional exemplary lyophilization methods can be found in U.S. Pat. Nos. 7,811,558, 8,486,617, and 8,097,403. An exemplary spray-drying method includes: combining nitrogen, as a drying gas, with a loading buffer according to the present disclosure, then introducing the mixture into GEA Mobile Minor spray dryer from GEA Processing Engineering, Inc. (Columbia Md., USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

In some embodiments, the step of drying the cargo-loaded platelets that are obtained as disclosed herein, such as the step of freeze-drying the cargo-loaded platelets that are obtained as disclosed herein, comprises incubating the platelets with a lyophilizing agent (e.g., a non-reducing disaccharide). Accordingly, in some embodiments, the methods for preparing cargo-loaded platelets further comprise incubating the cargo-loaded (e.g., drug) platelets with a lyophilizing agent. In some embodiments, cargo-loaded platelets are stimulated (e.g., by any of the stimulating agents described herein) to generate cargo-loaded (e.g., drug) extracellular vesicles.

In some embodiments the lyophilizing agent is a saccharide. In some embodiments the saccharide is a disaccharide, such as a non-reducing disaccharide.

In some embodiments, the platelets are incubated with a lyophilizing agent for a sufficient amount of time and at a suitable temperature to load the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrrolidone (PVP), starch, and hydroxyethyl starch (HES). In some embodiments, exemplary lyophilizing agents can include a high molecular weight polymer, into the loading composition. By “high molecular weight” it is meant a polymer having an average molecular weight of about or above 70 kDa. Non-limiting examples are polymers of sucrose and epichlorohydrin. In some embodiments, the lyophilizing agent is polysucrose. Although any amount of high molecular weight polymer can be used as a lyophilizing agent, it is preferred that an amount be used that achieves a final concentration of about 3% to 15% (w/v), such as 5% to 10%, for example 7%. In some embodiments, the lyophilizing agent (e.g., saccharides) are stabilization agents. For example, the stabilization agent can stabilize the platelet-derived extracellular vesicles as measured by platelet-specific cell surface marker expression and intact extracellular vesicles.

In some embodiments, the process for preparing a composition includes adding an organic solvent, such as ethanol, to the loading solution. In such a loading solution, the solvent can range from 0.1% to 5.0% (v/v). D

Within the process provided herein for making the compositions provided herein, addition of the lyophilizing agent can be the last step prior to drying. However, in some embodiments, the lyophilizing agent is added at the same time or before the drug, the cryoprotectant, or other components of the loading composition. In some embodiments, the lyophilizing agent is added to the loading solution, thoroughly mixed to form a drying solution, dispensed into a drying vessel (e.g., a glass or plastic serum vial, a lyophilization bag), and subjected to conditions that allow for drying of the solution to form a dried composition.

An exemplary saccharide for use in the compositions disclosed herein is trehalose. Regardless of the identity of the saccharide, it can be present in the composition in any suitable amount. For example, it can be present in an amount of 1 mM to 1 M. In some embodiments, it is present in an amount of from 10 mM 10 to 500 mM. In some embodiments, it is present in an amount of from 20 mM to 200 mM. In some embodiments, it is present in an amount from 40 mM to 100 mM. In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

The step of incubating the platelets to load them with a cryoprotectant or as a lyophilizing agent includes incubating the platelets for a time suitable for loading, as long as the time, taken in conjunction with the temperature, is sufficient for the cryoprotectant or lyophilizing agent to come into contact with the platelets and, preferably, be incorporated, at least to some extent, into the platelets. In some embodiments, incubation is carried out for about 1 minute to about 180 minutes or longer.

The step of incubating the platelets to load them with a cryoprotectant or lyophilizing agent includes incubating the platelets and the cryoprotectant at a temperature that, when selected in conjunction with the amount of time allotted for loading, is suitable for loading. In general, the composition is incubated at a temperature above freezing for at least a sufficient time for the cryoprotectant or lyophilizing agent to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

In various embodiments, the bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SA/V ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6 cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SA/V ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, the lyophilizing agent as disclosed herein may be a high molecular weight polymer. By “high molecular weight” it is meant a polymer having an average molecular weight of about or above 70 kDa and up to 1,000,000 kDa Non-limiting examples are polymers of sucrose and epichlorohydrin (polysucrose). Although any amount of high molecular weight polymer can be used, it is preferred that an amount be used that achieves a final concentration of about 3% to 10% (w/v), such as 3% to 7%, for example 6%. Other non-limiting examples of lyoprotectants are serum albumin, dextran, polyvinyl pyrrolidone (PVP), starch, and hydroxyethyl starch (HES).

In some embodiments, the lyophilized stabilized platelet derived extracellular vesicles can be at a concentration from about 1×10⁸ particles/mL to about 1×10¹⁶ particles/mL. In some embodiments, the lyophilized stabilized platelet derived extracellular vesicles can be at a concentration from about 1×10⁹ particles/mL to about 1×10¹⁵ particles/mL. In some embodiments, the lyophilized stabilized platelet derived extracellular vesicles can be at a concentration from about 1×10¹⁰ particles/mL to about 1×10¹⁴. In some embodiments, the lyophilized stabilized platelet derived extracellular vesicles can be at a concentration from about 1×10¹¹ particles/mL to about 1×10¹³. In some embodiments, the lyophilized stabilized platelet derived extracellular vesicles of about 1×10¹² particles/mL. In some embodiments, the therapeutically effective amount of lyophilized stabilized platelet derived extracellular vesicles can be at any of the concentrations described herein.

In some embodiments, the cargo loaded extracellular vesicles prepared as disclosed herein have a storage stability that is at least about equal to that of the extracellular vesicles prior to the loading of cargo (e.g., a drug).

The loading buffer may be any buffer that is non-toxic to the platelets and provides adequate buffering capacity to the solution at the temperatures at which the solution will be exposed during the process provided herein. Thus, the buffer may comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TBS). Likewise, it may comprise one or more of the following buffers: propane-1,2,3-tricarboxylic (tricarballylic); benzenepentacarboxylic; maleic; 2,2-dimethylsuccinic; EDTA; 3,3-dimethylglutaric; bis(2-hydroxyethyl)imino tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES).

In some embodiments the dried extracellular vesicles (such as freeze-dried extracellular vesicles) retain the loaded drug upon rehydration and release the drug upon stimulation by endogenous activators. In some embodiments at least about 10%, such as at least about 20%, such as at least about 30% of the drug is retained. In some embodiments from about 10% to about 20%, such as from about 20% to about 30% of the drug is retained.

In some embodiments, stabilized platelet derived extracellular vesicles generated by contacting platelets with a stimulating agent include a compositions from at least about 50% to at least about 95% stabilized platelet derived extracellular vesicles. In some embodiments, stabilized platelet derived extracellular vesicles generated by contacting platelets with a stimulating agent include a compositions from at least about 55% to at least about 90% stabilized platelet derived extracellular vesicles. In some embodiments, stabilized platelet derived extracellular vesicles generated by contacting platelets with a stimulating agent include a compositions from at least about 60% to at least about 80% stabilized platelet derived extracellular vesicles. In some embodiments, stabilized platelet derived extracellular vesicles generated by contacting platelets with a stimulating agent include a compositions from at least about 65% to at least about 75% stabilized platelet derived extracellular vesicles.

In some embodiments, stabilized platelet derived extracellular vesicles generated by contacting platelets with a stimulating agent include a compositions at least about 50% stabilized platelet derived extracellular vesicles. In some embodiments, stabilized platelet derived extracellular vesicles generated by contacting platelets with a stimulating agent include a compositions at least about 55% stabilized platelet derived extracellular vesicles. In some embodiments, stabilized platelet derived extracellular vesicles generated by contacting platelets with a stimulating agent include a compositions at least about 60% stabilized platelet derived extracellular vesicles. In some embodiments, stabilized platelet derived extracellular vesicles generated by contacting platelets with a stimulating agent include a compositions at least about 65% stabilized platelet derived extracellular vesicles. In some embodiments, stabilized platelet derived extracellular vesicles generated by contacting platelets with a stimulating agent include a compositions at least about 70% stabilized platelet derived extracellular vesicles. In some embodiments, stabilized platelet derived extracellular vesicles generated by contacting platelets with a stimulating agent include a compositions at least about 75% stabilized platelet derived extracellular vesicles. In some embodiments, stabilized platelet derived extracellular vesicles generated by contacting platelets with a stimulating agent include a compositions at least about 80% stabilized platelet derived extracellular vesicles. In some embodiments, stabilized platelet derived extracellular vesicles generated by contacting platelets with a stimulating agent include a compositions at least about 85% stabilized platelet derived extracellular vesicles. In some embodiments, stabilized platelet derived extracellular vesicles generated by contacting platelets with a stimulating agent include a compositions at least about 90% stabilized platelet derived extracellular vesicles.

As used in the compositions herein, “particle count” refers to the total count of extracellular vesicles (e.g., any of the platelet-derived extracellular vesicles described herein).

In some embodiments, the particle count in the composition is a particle count sufficient to generate from about 1 nM to about 1000 nM of thrombin in a thrombin generation assay. In some embodiments, the particle count in the composition is a particle count sufficient to generate from about 5 nM to about 900 nM of thrombin in a thrombin generation assay. In some embodiments, the particle count in the composition is a particle count sufficient to generate from about 10 nM to about 800 nM of thrombin in a thrombin generation assay. In some embodiments, the particle count in the composition is a particle count sufficient to generate from about 15 nM to about 700 nM of thrombin in a thrombin generation assay. In some embodiments, the particle count in the composition is a particle count sufficient to generate from about 20 nM to about 600 nM of thrombin in a thrombin generation assay. In some embodiments, the particle count in the composition is a particle count sufficient to generate about 25 nM to about 500 nM of thrombin in a thrombin generation assay. In some embodiments, the particle count in the composition is a particle count sufficient to generate about 30 nM to about 400 nM of thrombin in a thrombin generation assay. In some embodiments, the particle count in the composition is a particle count sufficient to generate about 35 nM to about 300 nM of thrombin in a thrombin generation assay. In some embodiments, the particle count in the composition is a particle count sufficient to generate about 40 nM to about 200 nM of thrombin in a thrombin generation assay. In some embodiments, the particle count in the composition is a particle count sufficient to generate about 45 nM to about 100 nM of thrombin in a thrombin generation assay. In some embodiments, the particle count in the composition is a particle count sufficient to generate about 50 nM in a thrombin generation assay.

In some embodiments, a composition comprising stabilized platelet derived extracellular vesicles have an osmolarity (mOsm) of at least about 300 mOsm, at least about 350 mOsm, at least about 400 mOsm, at least about 450 mOsm, at least about 500 mOsm, at least about 550 mOsm, at least about 600 mOsm, at least about 650 mOsm, at least about 700 mOsm, at least about 750 mOsm, at least about 800 mOsm, at least about 850 mOsm, at least about 900 mOsm, at least about 950 mOsm, or at least about 1000 mOsm.

In some embodiments, a composition comprising stabilized platelet derived extracellular vesicles have an osmolarity (mOsm) of less than about 300 mOsm, less than about 350 mOsm, less than about 400 mOsm, less than about 450 mOsm, less than about 500 mOsm, less than about 550 mOsm, less than about 600 mOsm, less than about 650 mOsm, less than about 700 mOsm, less than about 750 mOsm, less than about 800 mOsm, less than about 850 mOsm, less than about 900 mOsm, less than about 950 mOsm, or less than about 1000 mOsm.

In some embodiments, a composition comprising stabilized platelet derived extracellular vesicles have a viscosity at 37° C. as measured by centipoise (cP) of at least about 1.0, at least about 1.1, at least about 1.2, at least about 1.3, at least about 1.4, at least about 1.5, at least about 1.6, at least about 1.7, at least about 1.8, at least about 1.9, or at least about 2.0.

In some embodiments, a composition comprising stabilized platelet derived extracellular vesicles have a viscosity at 37° C. as measured by centipoise (cP) of less than about 1.0, less than about 1.1, less than about 1.2, at less than about 1.3, less than about 1.4, less than about 1.5, less than about 1.6, less than about 1.7, less than about 1.8, less than about 1.9, or less than about 2.0.

In some embodiments, a composition comprising stabilized platelet derived extracellular vesicles have a diameter (nm) as measured by nanoparticle tracking analysis of at least about 120 nm, at least about 125 nm, at least about 130 nm, at least about 135 nm, at least about 140 nm, at least about 145 nm, at least about 150 nm, at least about 155 nm, at least about 160 nm, at least about 165 nm, at least about 170 nm, at least about 175 nm, at least about 180 nm, at least about 185 nm, at least about 190 nm, at least about 195 nm, or at least about 200 nm. In some embodiments, the composition comprising stabilized platelet derived extracellular vesicles is lyophilized.

In some embodiments, a composition comprising stabilized platelet derived extracellular vesicles have a diameter (nm) as measured by nanoparticle tracking analysis of less than about 120 nm, less than about 125 nm, less than about 130 nm, less than about 135 nm, less than about 140 nm, less than about 145 nm, less than about 150 nm, less than about 155 nm, less than about 160 nm, less than about 165 nm, less than about 170 nm, less than about 175 nm, less than about 180 nm, less than about 185 nm, less than about 190 nm, less than about 195 nm, or less than about 200 nm. In some embodiments, the composition comprising stabilized platelet derived extracellular vesicles is lyophilized.

In some embodiments, a composition comprising stabilized platelet derived extracellular vesicles have nanoparticle tracking analysis concentration (particles/mL) of at least about 4.0×10¹² (particles/mL), at least about 5.0×10¹² (particles/mL), at least about 1.0×10¹³ (particles/mL), at least about 2.0×10¹³ (particles/mL), at least about 3.0×10¹³ (particles/mL), at least about 4.0×10¹³ (particles/mL), or at least about 5.0×10¹³ (particles/mL).

In some embodiments, a composition comprising stabilized platelet derived extracellular vesicles have nanoparticle tracking analysis concentration (particles/mL) of less than about 4.0×10¹² (particles/mL), less than about 5.0×10¹² (particles/mL), less than about 1.0×10¹³ (particles/mL), less than about 2.0×10¹³ (particles/mL), less than about 3.0×10¹³ (particles/mL), less than about 4.0×10¹³ (particles/mL), or less than about 5.0×10¹³ (particles/mL).

In some embodiments, a composition comprising stabilized platelet derived extracellular vesicles have mean diameter, measured by a thrombolux assay, of at least about 120 nm, at least about 125 nm, at least about 130 nm, at least about 135 nm, at least about 140 nm, at least about 145 nm, at least about 150 nm, at least about 155 nm, at least about 160 nm, at least about 165 nm, at least about 170 nm, at least about 175 nm, at least about 180 nm, at least about 185 nm, at least about 190 nm, at least about 195 nm, at least about 200 nm, at least about 205 nm, at least about 210 nm, at least about 215 nm, at least about 220 nm, at least about 225 nm, at least about 230 nm, at least about 235 nm, at least about 240 nm, at least about 245 nm, at least about 250 nm, at least about 255 nm, at least about 260 nm, at least about 265 nm, at least about 275 nm, at least about 280 nm, at least about 285 nm, at least about 290 nm, at least about 295 nm, or at least about 300 nm.

In some embodiments, a composition comprising stabilized platelet derived extracellular vesicles have mean diameter, measured by a thrombolux assay, of less than about 120 nm, less than about 125 nm, less than about 130 nm, less than about 135 nm, less than about 140 nm, less than about 145 nm, less than about 150 nm, less than about 155 nm, less than about 160 nm, less than about 165 nm, less than about 170 nm, less than about 175 nm, less than about 180 nm, less than about 185 nm, less than about 190 nm, less than about 195 nm, less than about 200 nm, less than about 205 nm, less than about 210 nm, less than about 215 nm, less than about 220 nm, less than about 225 nm, less than about 230 nm, less than about 235 nm, less than about 240 nm, less than about 245 nm, less than about 250 nm, less than about 255 nm, less than about 260 nm, less than about 265 nm, less than about 275 nm, less than about 280 nm, less than about 285 nm, less than about 290 nm, less than about 295 nm, or less than about 300 nm.

In some embodiments, a composition comprising stabilized platelet derived extracellular vesicles have a scattering intensity, measured by a thrombolux assay, of at least about 40 kHz, of at least about 45 kHz, of at least about 50 kHz, of at least about 55 kHz, of at least about 60 kHz, of at least about 65 kHz, of at least about 70 kHz, of at least about 75 kHz, of at least about 80 kHz, of at least about 85 kHz, of at least about 90 kHz, of at least about 95 kHz, of at least about 100 kHz, of at least about 105 kHz, of at least about 110 kHz, of at least about 115 kHz, of at least about 120 kHz, of at least about 125 kHz, of at least about 130 kHz, of at least about 135 kHz, of at least about 140 kHz, of at least about 145 kHz, or of at least about 150 kHz.

In some embodiments, a composition comprising stabilized platelet derived extracellular vesicles have a scattering intensity, measured by a thrombolux assay, of less than about 40 kHz, of less than about 45 kHz, of less than about 50 kHz, of less than about 55 kHz, of less than about 60 kHz, of less than about 65 kHz, of less than about 70 kHz, of less than about 75 kHz, of less than about 80 kHz, of less than about 85 kHz, of less than about 90 kHz, of less than about 95 kHz, of less than about 100 kHz, of less than about 105 kHz, of less than about 110 kHz, of less than about 115 kHz, of less than about 120 kHz, of less than about 125 kHz, of less than about 130 kHz, of less than about 135 kHz, of less than about 140 kHz, of less than about 145 kHz, or of less than about 150 kHz.

A thrombin generation assay may be, for example, an assay as disclosed in Hemker, H. et al., Calibrated Automated Thrombin Generation Measurement in Clotting Plasma, Pathophysiol Haemost Thromb. 2003, 33:4-15. Hemker et al. is incorporated by reference herein in its entirety.

In some embodiments, a total thrombus-formation analysis system (T-TAS®) assay (also referred to as adhesion to collagen and generation of fibrin under flow assay) is performed to measure thrombus formation (e.g., clot formation) under physiological conditions (e.g., shear flow).

In some embodiments, a T-TAS® may be, for example, any assay disclosed at https://www.t-tas.info/pub/, incorporated by reference herein. For example, a T-TAS assay may be one or more of the following, each of which is incorporated by reference herein in its entirety: Al Ghaithi, R, Evaluation of the Total Thrombus-Formation System (T-TAS), Platelets, No. 42 (2018); Taune, V., Whole blood coagulation assays ROTEM and T-TAS to monitor dabigatran t dabigatran treatment, Thrombosis Research, No. 30 (2017); Daidone, V., Usefulness of the Total Thrombus-formation Analysis System (T-TAS) in the diagnosis and characterization of von Willebrand disease, Haemophilia, No. 20 (2016); Ito, M., Total Thrombus-Formation Analysis System (T-TAS) Can Predict Periprocedural Bleeding Events in Patients Undergoing Catheter Ablation for Atrial Fibrillation, Journal of American Heart Association, No. 16 (2015).

In some embodiments, the particle count in the composition is a particle count sufficient to produce an occlusion time of less than 30 minutes in a total thrombus-formation analysis system (T-TAS) assay. In some embodiments, the particle count in the composition is a particle count sufficient to produce an occlusion time of less than 25 minutes in a total thrombus-formation analysis system (T-TAS) assay. In some embodiments, the particle count in the composition is a particle count sufficient to produce an occlusion time of less than 20 minutes in a total thrombus-formation analysis system (T-TAS) assay. In some embodiments, the particle count in the composition is a particle count sufficient to produce an occlusion time of less than 15 minutes in a total thrombus-formation analysis system (T-TAS) assay. In some embodiments, the particle count in the composition is a particle count sufficient to produce an occlusion time of less than 10 minutes in a total thrombus-formation analysis system (T-TAS) assay. In some embodiments, the particle count in the composition is a particle count sufficient to produce an occlusion time of less than 5 minutes in a total thrombus-formation analysis system (T-TAS) assay. In some embodiments, the particle count in the composition is a particle count sufficient to produce an occlusion time of less than 30, 29, 28, 27, 26, 25, 24, 23, 22, 21, 20, 19, 18, 17, 16, 16, 15, 14, 13, 12, 11, 10, 9, 8, 7, 6, 5, 4, 3, 2, or 1 minute(s) in a total thrombus-formation analysis assay.

In some embodiments, the cargo-loaded (e.g., drug) extracellular vesicles retain the loaded drug compound upon rehydration and release the drug compound upon stimulation by endogenous activators, such as endogenous activators described herein. In some embodiments, the cargo is a drug. In some embodiments, the drug is a drug to treat cancer. In some embodiments, a therapeutically effective amount of cargo-loaded (e.g., drug) stabilized extracellular vesicles are used to treat a subject with cancer.

In some embodiments, provided herein is a method of treating bleeding (e.g., hemorrhage) in a subject. For example, extracellular vesicles (e.g., extracellular vesicles derived from any of the platelets described herein, including thrombosomes) can possess similar procoagulant activity to platelets, such as for example, during hemostasis, or arrest of bleeding. In some embodiments, a therapeutically effective amount of stabilized extracellular vesicles is used to treat bleeding in a subject.

In some embodiments, provided herein is a method of treating a wound in a subject. For example, extracellular vesicles (e.g., extracellular vesicles derived from any of the platelets described herein) can possess similar activity as platelets, such as for example, during wound healing. In some embodiments, a therapeutically effective amount of stabilized extracellular vesicles is used to treat a wound in a subject.

### EXAMPLES

### Example 1. Preparing Stabilized Platelet-Derived Extracellular Vesicles

Platelets were collected, pooled, and stimulated with either a calcium ionophore (A23187 (Calcimycin)) or a thrombin collagen receptor antagonist (Concluxin or collagen related peptide mixtures) to generate platelet-derived extracellular vesicles. Platelet-derived extracellular vesicles were isolated by ultracentrifugation at 100,000 g for 60 minutes at 4° C. or the platelet components were removed and the remaining extracellular vesicle containing solution was filtered through a 0.45 μm syringe filter to collect platelet derived extracellular vesicles. The extracellular vesicles were stabilized either in loading buffer 1 (Table 1) or in loading buffer 2 (PBS buffer (pH approximately between 6.6-6.8 titrated with acid-citrate-dextrose (A.C.D)) including 7% sucrose (or, alternatively polysucrose) and 2% trehalose).

TABLE 1
Loading Buffer 1

| | |
|---|---|
| NaCl | 750 |
| KCl | 48 |
| HEPES | 95 |
| NaHCO₃ | 120 |
| Dextrose | 3 |
| Trehalose | 0.1M |
| Ethanol | 1.0% (v/v) |
| (optional) | |

### Example 2—Preparing Lyophilized Extracellular Vesicles

Stabilized platelet-derived extracellular vesicles prepared in Example 1 were lyophilized (e.g., freeze dried) according to the following protocol.

Protocol 1: Lyophilization Protocol

Freezing:

Step 1: Ramp up to −40° C. for 0 minutes.

Step 2: Incubate the extracellular vesicles at −40° C. for 180 minutes.

Final Freezing:

Incubate at −40° C. for 0 minutes; pressure at 0 mT.

Primary Drying:

Step 1: Ramp to −10° C. for 360 minutes at a rate of 5° C./hr

Step 2: Hold at −10° C. for 540 minutes

Step 3: Ramp to +5° C. for 180 minutes at a rate of 5° C./hr

Step 4: Hold at +5° C. for 540 minutes

Step 3: Ramp to +30° C. for 300 minutes at a rate of 5 C/hr

Secondary Drying:

Step 1: Incubate at 30° C. for 720 minutes; pressure at 0 mT.

Step 2: Incubate at 30° C. for 720 minutes; pressure at 200 mT.

Step 3: Incubate at 30° C. at 9999 minutes; pressure at 0 mT. Hold for a minimum of 60 minutes.

### Example 3—Fresh, Frozen, and Lyophilized Extracellular Vesicles Exhibit Platelet-Like Properties

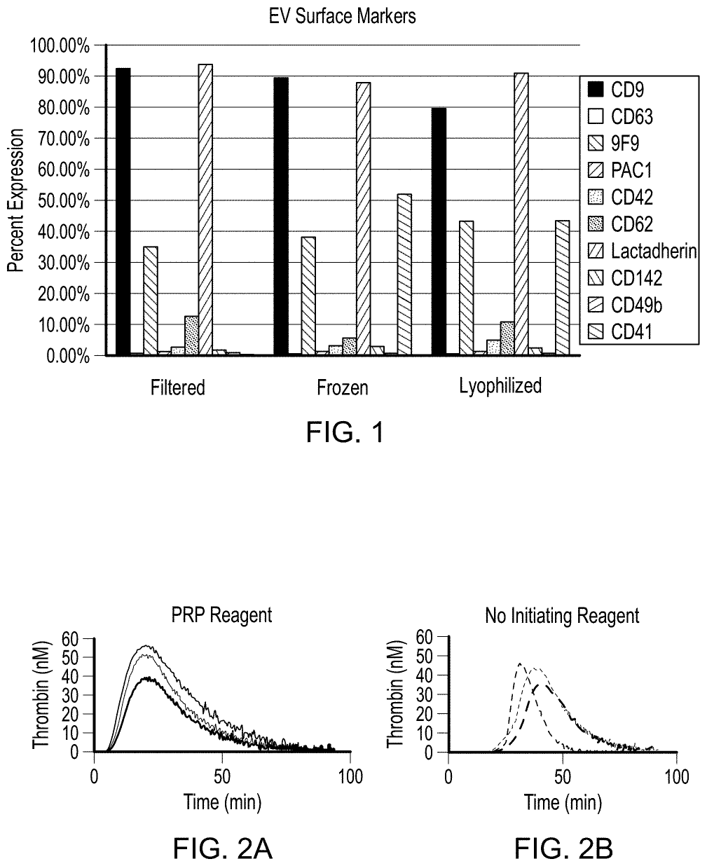

FIG. 1 shows percent expression of platelet-derived extracellular vesicle surface marker expression such as CD9, CD63, 9F9, PAC1, CD42, CD62, Lactadherin, CD142, CD49b, and CD41. The data show activated platelet-specific marker expression such as CD41, CD62, and 9F9, as well as exosome-specific markers, such as CD9 on platelet-derived extracellular vesicles from filtered (fresh) platelets and frozen platelets as described in Example 1, as well as, lyophilized extracellular vesicles prepared as described in Example 2. Comparing surface marker expression between the sample preparation methods (e.g., filtered, frozen, and lyophilized extracellular vesicles), shows that surface marker expression is comparable regardless of the preparation process, demonstrating the stability of stabilized platelet-derived extracellular vesicles.

Table 2 below shows protein concentration inside the extracellular vesicles (EVs) from extracellular vesicles along the preparation process. To determine the internal protein content, the extracellular vesicles were lysed and measured via micro-BCA and in some cases for total phospholipid (data not shown). Table 2 shows the internal total protein concentration in the lyophilized extracellular vesicles is 370 μg/ml which exceeds the total protein concentration of either extracellular vesicles prepared from either filtered (fresh) or frozen platelets.

TABLE 2
Internal extracellular vesicle protein concentration.

| | Protein Concentration |
|---|---|
| Sample | (μg/mL) |
| Liquid filtered EVs | 316 |
| Frozen EVs | 364 |
| Lyophilized EVs | 370 |

### Example 4—Stabilized Platelet-Derived Extracellular Vesicle Characteristics

Table 3 below summarizes extracellular vesicle yield (e.g., concentration (particles/mL)), mean particle size (nm), and extracellular vesicles per platelet for lyophilized EVs analyzed under various conditions with a nanoparticle tracking analysis (NTA) device (Nanosight, https://www.malvernpanalytical.com/en/products/product-range/nanosight-range). The sample with the highest extracellular vesicle concentration (particles/mL) and with the highest exosome content (e.g., extracellular vesicles from approximately 40 nM to 200 nM) was from the 2-day post collection in plasma (no manipulation) sample, followed by the 7-day post collection in plasma sample. Extracellular vesicle production, in terms of size and yield, is highly dependent on the method of stimulation. For example, following lyophilization, a drop in extracellular vesicle yield was observed, however, extracellular loss can be minimized given the appropriate conditions. Loading buffer 2 (PBS and either 7% sucrose or polysucrose and 2% trehalose) resulted in a higher extracellular vesicle yield and more exosome content than loading buffer 1 (Table 1). Below is a summary of the results.

TABLE 3
Lyophilized extracellular vesicle size and yield per stimulation method

| Stimulation | Suspension | Stabilization | EV Yield | Mean Size | EVs per |
|---|---|---|---|---|---|
| Method | Medium | Method | (Particles/ml) | (nm) | Platelet |
| Aged 2-day | Plasma | Lyophilized | 1.75E+14 | 88.5 | 1.09E+05 |
| post collection | | | | | |
| Aged 7-day | Plasma | Lyophilized | 6.91E+13 | 121.5 | 5.42E+04 |
| post collection | | | | | |
| Calcium | Buffer 1 | Lyophilized | 5.91E+09 | 195.6 | 2.96E+00 |
| Ionophore | | | | | |
| Calcium | Buffer 2 | Lyophilized | 9.83E+10 | 185.1 | 5.65E+01 |
| Ionophore | | | | | |

A major platelet function is the ability to generate thrombin necessary for clot formation. Extracellular vesicles derived from platelets were tested for their ability to generate thrombin. Extracellular vesicles were either loaded with a fluorescent dye (carboxyfluorescein diacetate (CFDA)) or were left unloaded and were evaluated for their ability to generate thrombin in vitro using a calibrated automated thrombogram (CAT). In the presence of an initiating reagent platelet rich plasma (PRP), both extracellular vesicles loaded with CFDA and unloaded extracellular vesicles generated thrombin (FIG. 2A). In the absence of an initiating reagent (PRP), the extracellular vesicles generated significantly more thrombin, shown by endogenous thrombin potential (ETP), which corresponds to the area under the curve (FIG. 2B). In both FIGS. 2A and 2B the negative control, Octaplas sample, did not generate thrombin.

Table 4 below summarizes the thrombin generation assay (TGA) results from fresh, frozen, and lyophilized loaded (CFDA) extracellular vesicles and unloaded extracellular vesicles without a stimulating agent during the stabilization process. By comparing comparable samples (e.g., Fresh EVs (unloaded) to Fresh CFDA EVs (loaded), etc.) the data show that extracellular vesicles retain similar or more robust thrombin generation function as unloaded extracellular vesicles as measured by ETP (nM/min), ETP/Protein (nM/min)/μg, Thrombin Peak (nM), Thrombin Peak/Protein (nM/μg), and Lag Tim (min)

TABLE 4
Thrombin generation capabilities of extracellular vesicles without
a stimulating reagent along the stabilization process.

| | ETP | ETP/Protein | | Thrombin | Lag |
|---|---|---|---|---|---|
| | (nM · | (nM · | Thrombin | Peak/Protein | Time |
| | min) | min)/μg | Peak (nM) | (nM/μg) | (min) |
| Fresh EVs | 866 | 2887 | 36 | 120 | 28.4 |
| Frozen EVs | 688 | 2293 | 32 | 107 | 32.3 |
| Lyophilized | 787 | 2623 | 35 | 117 | 24.3 |
| EVs | | | | | |
| Fresh | 1098 | 3660 | 44 | 147 | 24.0 |
| CFDA EVs | | | | | |
| Frozen | 904 | 3013 | 42 | 140 | 39.0 |
| CFDA EVs | | | | | |
| Lyophilized | 848 | 2827 | 38 | 127 | 27.4 |
| CFDA EV | | | | | |

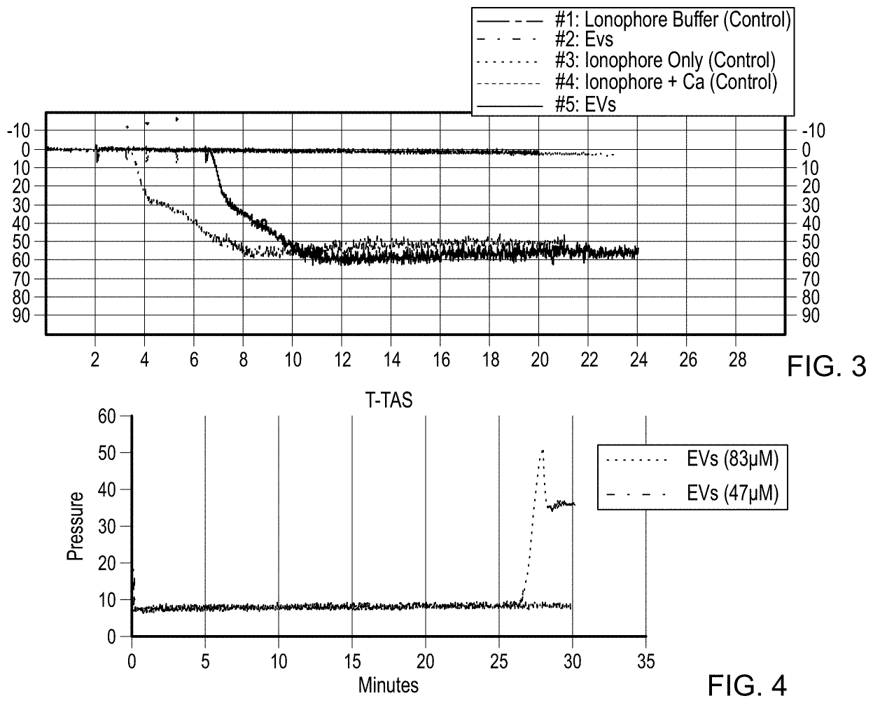

Another major platelet function is the ability to aggregate during primary hemostasis. Extracellular vesicles were assessed for their capacity to promote platelet aggregation in the absence of an agonist. Extracellular vesicles were added in a 1:10 dilution to freshly drawn platelet rich plasma. As shown in FIG. 3, Channel 1 contained the extracellular buffer (Ionophore) used to stimulate the platelets to generate the extracellular vesicles, but without the extracellular vesicles as a control. Channel 2 contained extracellular vesicles. Channel 3 contained the Ionophore stimulus (Ca¹²) used to generate the extracellular vesicles as a control. Channel 4 contained another control similar to Channel 1, however, with the addition of calcium, and Channel 5 was a duplicate run of the extracellular vesicle sample (Channel 2).

FIG. 3 demonstrates the ability of stabilized platelet-derived extracellular vesicles to aggregate in the absence of an agonist as compared to control samples (Channels 1, 3, and 4). The area under the curve (AUC) for the extracellular vesicle samples were 845 and 914 (Channels 2 and 5), while the area under the curve for the control samples ranged from 19 to 29. Similarly, the percent aggregation for the extracellular vesicles samples (Channels 2 and 5) were 57 and 60 percent aggregation while the control samples were between 2-3 percent aggregation (Channels 1, 3, and 4).

Extracellular vesicles were also assessed for their ability to adhere to collagen under shear flow conditions, which simulate adhesion to injured endothelium under physiological conditions on a total thrombus-formation analysis system (T-TAS®). The extracellular vesicles were diluted 1:10 into Octaplas (50 μL into 450 μL of Octaplas). This mixture was run across a chip (AR chip) coated with collagen and tissue factor at high vascular shear simulating physiological conditions. The sample was run for 30 minutes. Back pressure was recorded and indicated which channel was occluded which is indicative of clot formation. FIG. 4 shows two samples run (extracellular vesicles at a concentration of 47 μM and 83 μM). The 83 μM sample occluded at approximately 26 minutes which is indicative of clot formation, while the other sample, 47 μM, that had approximately half the concentration (as measured by the phospholipid content) did not occlude, indicating no clot formation. The different responses suggest that an appropriate dose should be selected to elicit a potent occlusion (clot formation) response.

### Example 5—Stabilized Platelet-Derived Extracellular Vesicles as a Cargo Delivery Mechanism

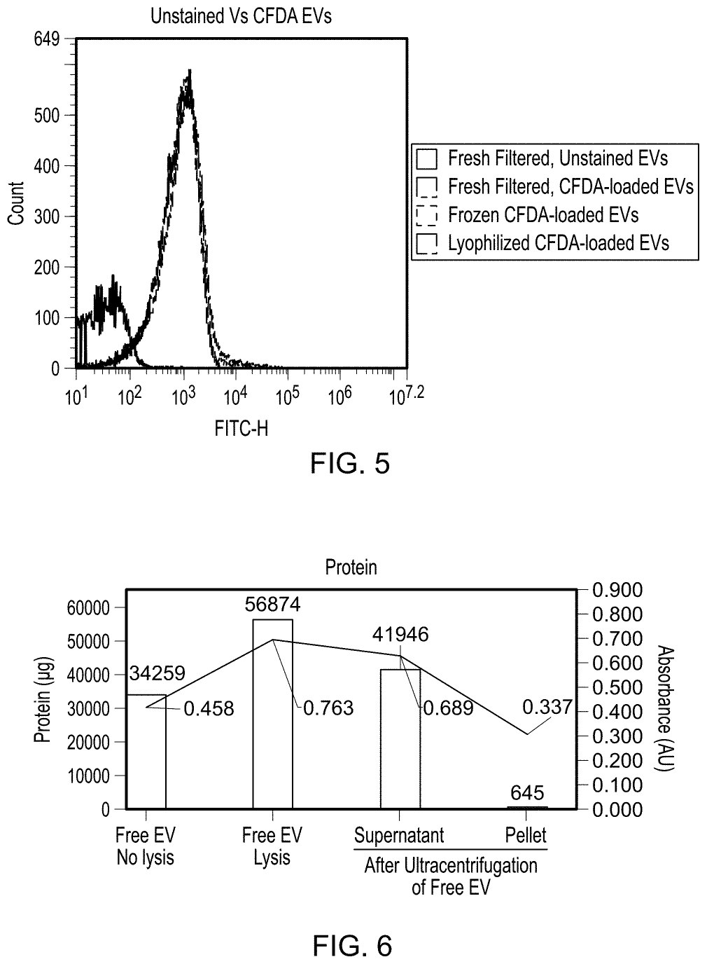

Extracellular vesicles were also evaluated for their ability to serve for drug delivery applications (e.g., cancer). Extracellular vesicles prepared from fresh and frozen platelets were loaded with a fluorescent dye, carboxyfluorescein diacetate succinimidyl ester (CFDA), and subjected to the lyophilization and stabilization process generating lyophilized CFDA-loaded extracellular vesicles. During the lyophilization and stabilization process the mean fluorescent intensity (MFI) of the CFDA dye was measured using flow cytometry. As a control, extracellular vesicles that were unstained and unloaded with CFDA were also run. FIG. 5 shows that following lyophilization a slight decrease in MFI was observed, indicating that the extracellular vesicle membrane was still intact and retained the CFDA dye internally. These data demonstrate that extracellular vesicles prepared from fresh and frozen platelets, loaded with cargo, and subsequently lyophilized and stabilized can be loaded with any target cargo, stabilized, and used as a vehicle for drug delivery applications.

### Example 6—Stabilized Platelet-Derived Extracellular Vesicles from Thrombosomes

Extracellular vesicles derived from thrombosomes (lyophilized platelets) were generated by centrifuging thrombosomes (1000 K/μl) at 18,000 g for 90 seconds (fraction “A”). After centrifugation the supernatant (Fraction “B”) including the extracellular vesicles was retained and the thrombosome pellet (Fraction “C”) was discarded. Fraction B was ultracentrifuged at 100,000 g for 60 minutes at 4° C. resulting in supernatant (Fraction “D”) and thrombosome-derived extracellular vesicles (Fraction “E”).

FIG. 6 is a graph showing the results of a micro bicinchonic acid (BCA) assay measuring protein concentration (μg) from different fractions during the thrombosome generated extracellular vesicle process. From left to right, the vertical bars in FIG. 6 represent Fraction B, Fraction C, Fraction D, and Fraction E, respectively. The micro BCA assay showed a difference in protein concentration between un-lysed (Fraction B) and lysed free extracellular vesicle samples (Fraction C). After ultracentrifugation the protein concentration as measured by the micro BSA assay was significantly higher in Fraction C (supernatant) than in Fraction D (thrombosome-derived extracellular vesicles).

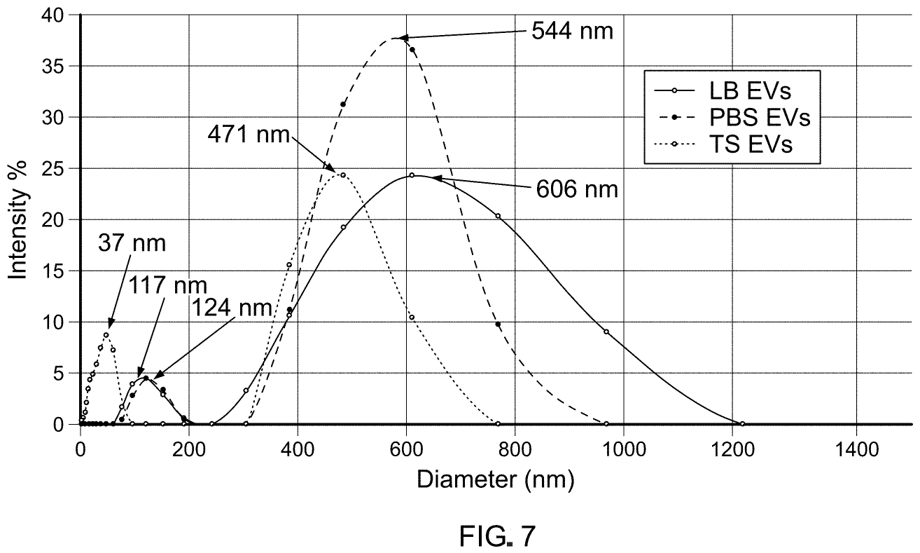

FIG. 7 is a graph showing the results of extracellular vesicles generated with a calcium ionophore stimulating agent in either buffer 1 or buffer 2 as compared with thrombosome generated extracellular vesicles. FIG. 7 shows the % intensity×diameter (nm) for platelet derived extracellular vesicles stabilized in buffer 1 (LB EVs) or buffer 2 (PBS EVs) and thrombosome derived extracellular vesicles (TS EVs). Table 5 summarizes the average diameter, the polydispersity index, and the counts per second measured for each of the platelet-derived extracellular vesicles generated under different conditions.

TABLE 5

| | Average | | Counts per Second |
|---|---|---|---|
| | Diameter (nm) | PDI | (×10⁴) |
| LB EVs | 540 | 0.50 | 18.9 |
| PBS EVs | 497 | 0.38 | 85.6 |
| TS EVs | 257 | 0.88 | 15.5 |

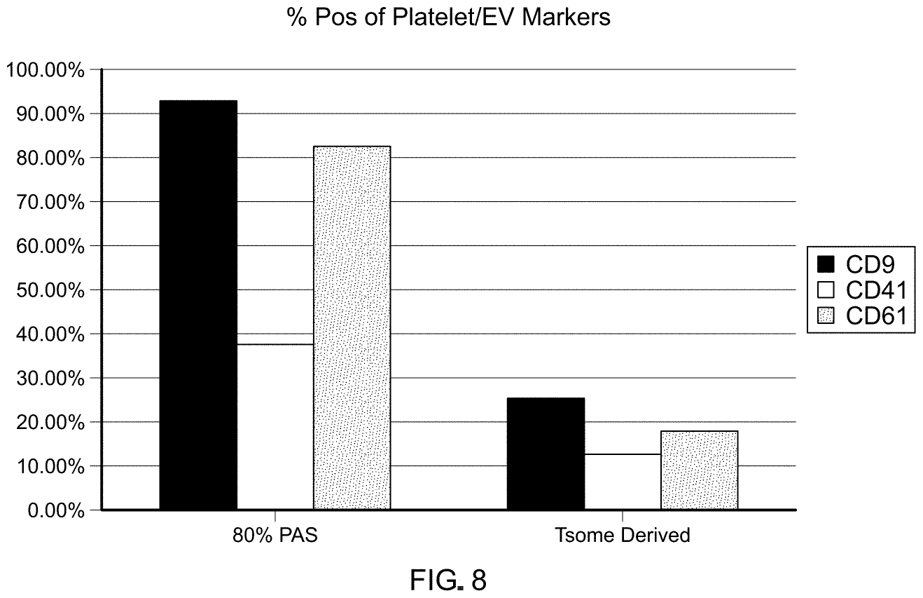

FIG. 8 is a graph showing the measurement of platelet-specific cell surface markers from platelet-derived extracellular vesicles (PAS) and thrombosome-derived extracellular vesicles (Tsome derived). Cell surface marker expression for CD9, CD41, and CD61 was measured. The data show that extracellular vesicles derived from thrombosomes retain platelet-specific cell surface marker expression.

The concentration of thrombosome derived extracellular vesicles was 4.4×10⁹ particles/mL with a 96% of particles<400 nm as measured by a NanoSight assay (data not shown).

### Example 7—Stabilized Platelet-Derived Extracellular Vesicles Display Platelet Specific Markers

Stabilized platelet-derived extracellular vesicles were dried and assayed for platelet factor 4 (PF4) marker.

Pooled platelet Apheresis Units at 2,000 cells/μl were stimulated with 10 μM of calcium ionophore A23187 (Calcimycin) for 30 min at 37° C. in PBS (pH 6.7) and intact platelets were removed by centrifugation at 2500×g for 20 minutes. Extracellular vesicles present in the supernatant were filtered through a 0.45 μm filter then stabilized with 7% sucrose and 2% trehalose (loading buffer 2) and lyophilized. The samples were rehydrated in 1 ml of sterile water and tested for total protein and for platelet factor 4 (PF4) by BCA assay using BSA standard or ELISA (ThermoScientific), before and after lysis in 2.5% Triton™ X-100 in PBS for 30 min at 37uC. Non-lysed samples only received PBS buffer. PF4 concentration of the extracellular vesicles was calculated as the difference between lysed and non-lysed samples. The dried extracellular vesicles contained between 8.1 and 896.4 ng of PF4 per extracellular vesicle as measured by an ELISA assay and comparing lysed extracellular vesicles to unlysed extracellular vesicles (data not shown).

### Example 8 Extracellular Vesicle Stimulation Conditions

The following experiments were performed to determine optimal vesicle stimulation (e.g., vesiculation) conditions for frozen extracellular vesicle samples. Platelet samples were frozen after either 2, 7, or 14 days post collection.

TABLE 6
EV Aging Study 2 - Non-irradiated APUs - TBX

| | | Mean | Mode | | Days Aged |
|---|---|---|---|---|---|
| | Conc. | Size | size | Storage | (Post- |
| Sample | (particles/ml) | (nm) | (nm) | Temperature | Collection) |
| R2 | 7.0E+11 | 68.1 | 63.1 | Room | Day 2 |
| | | | | Temp | |
| R7 | 6.9E+11 | 70.5 | 54.8 | Room | Day 7 |
| | | | | Temp | |
| RT14 | 1.1E+12 | 86.6 | 62.4 | Room | Day 14 |
| | | | | Temp | |
| CS2 | 1.4E+12 | 110.1 | 81.5 | Cold | Day 2 |
| | | | | Shocked | |
| CS7 | 1.3E+12 | 106.4 | 79.2 | Cold | Day 7 |
| | | | | Shocked | |
| CS14 | 9.8E+11 | 95.4 | 74.1 | Cold | Day 14 |
| | | | | Shocked | |
| C7 | 1.2E+12 | 76.3 | 62.1 | Cold | Day 7 |
| | | | | Stored | |
| C14 | 7.0E+11 | 88.3 | 78.2 | Cold | Day 14 |
| | | | | Stored | |

TABLE 7
EV Aging Study 3-Irradiated APUs-CPP

| | | Mean | | | Days Aged | |
|---|---|---|---|---|---|---|
| | Conc. | Size | Mode | Storage | (Post- | Filtration |
| Sample | (particles/ml) | (nm) | size (nm) | Temperature | Collection) | Information |
| R1UF | 2.2E+12 | 97.4 | 76.6 | Room Temp | Day 2 | unfiltered |
| R1F4 | 1.5E+12 | 73.4 | 60.7 | Room Temp | Day 2 | .45 μm syringe filter |
| R1F2 | 1.0E+12 | 72.9 | 69.5 | Room Temp | Day 2 | .22 μm filter flask |
| R3S | 1.9E+12 | 99.3 | 77.8 | Room Temp | Day 3 | .45 μm syringe filter |
| R3F | 2.3E+12 | 79.4 | 75 | Room Temp | Day 3 | .45 μm filter flask |
| R6F | 1.2E+12 | 91.2 | 78.8 | Room Temp | Day 6 | .45 μm filter flask |
| R14F | 7.5E+11 | 98.1 | 72.4 | Room Temp | Day 14 | .45 μm filter flask |
| C3S | 8.5E+11 | 79.9 | 63.7 | Cold Stored | Day 3 | .45 μm syringe filter |
| C3F | 3.2E+12 | 82.3 | 61.8 | Cold Stored | Day 3 | .45 μm filter flask |
| C6F | 8.0E+11 | 79.4 | 75 | Cold Stored | Day6 | .45 μm filter flask |
| C14F | 9.1E+11 | 76.9 | 70.4 | Cold Stored | Day 14 | .45 μm filter flask |

TABLE 8
EV Aging Study 2 - Non-irradiated APUs

| | | Mean | Mode | | Days Aged |
|---|---|---|---|---|---|
| | Conc. | Size | size | Storage | (Post- |
| Sample | (particles/ml) | (nm) | (nm) | Temperature | Collection) |
| CS2 | 1.4E+12 | 110.1 | 81.5 | Cold | Day 2 |
| | | | | Shocked | |
| CS14 | 9.8E+11 | 95.4 | 74.1 | Cold | Day 14 |
| | | | | Shocked | |

TABLE 9
EV Aging Study 3-Irradiated APUs

| | | Mean | Mode | | Days Aged | | |
|---|---|---|---|---|---|---|---|
| | Conc. | Size | size | Storage | (Post- | Filtration | |
| Sample | (particles/ml) | (nm) | (nm) | Temperature | Collection) | Information | Sample |
| R3F | 2.3E+12 | 79.4 | 75 | Room Temp | Day 3 | .45 um filter | R3F |
| | | | | | | flask | |
| R6F | 1.2E+12 | 91.2 | 78.8 | Room Temp | Day 6 | .45 um filter | R6F |
| | | | | | | flask | |
| R14F | 7.5E+11 | 98.1 | 72.4 | Room Temp | Day 14 | .45 um filter | R14F |
| | | | | | | flask | |
| C3F | 3.2E+12 | 82.3 | 61.8 | Cold Stored | Day 3 | .45 um filter | C3F |
| | | | | | | flask | |

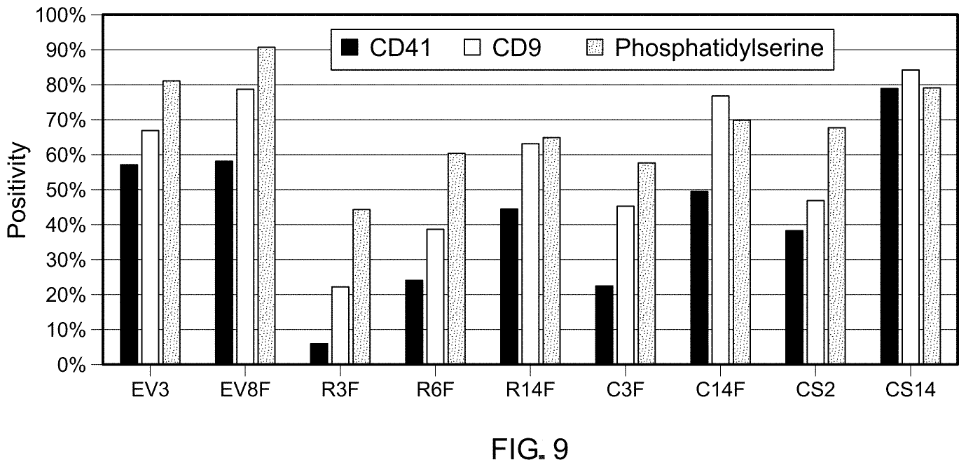

FIG. 9 shows percent positivity for CD41, CD9, and phosphatidylserine for 7-day aging in PAS unfiltered, ionophore filtered, 3-day aging room temperature filtered, 6-day aging at room temperature filtered, 14-day aging at room temperate filtered, 3-day aging at 4° C. filtered, 14-day aging at 4° C. filtered, 2-day aging at room temperature then cold shocked unfiltered, and 14-day aging at room temperature then cold shocked unfiltered samples. The data demonstrate that increased aging time results in increased percentages of platelet markers.

### Example 9. Extracellular Vesicle Tangential Flow Filtration (TFF)-Cryopreserved Plasma

Plasma from pooled cryopreserved platelet processing was irradiated (apheresis pooled units (APUs) (12 units, 7 donors)). During cryopreserved platelet processing, 2-unit pools of APUs were centrifuged at 1250 g for 10 minutes, plasma was then removed from platelet pellets. Post cryopreserved platelet processing all plasma was pooled into a 3L pooling bag. Next, 12 even aliquots (150-200 ml) of the plasma were aliquoted into 600 mL transfer bags. The aliquots then underwent a 2500 g centrifugation for 20 minutes, to pellet residual platelets. Plasma was then expressed. 6 of these platelet poor plasma (PPP) units were then pooled/filtered using a 1L, 0.45 μm filter flask and 6 filtered PPP units were then aliquoted into 600 mL transfer bags and frozen at −80° C.

Tangential flow filtration (TFF) was performed with a filter membrane: 500 kDa NMWCO. The extracellular vesicles were then incubated in loading buffer 2 as in Example 1 (e.g., PBS (acidified to pH 6.6-6.8 with ADC)) and then formulated to 7% sucrose, 2% trehalose (% w/w)). 150 mL of the starting material was put into TFF system and diluted with 350 mL of loading buffer 2 (500 mL initial dilution). Pressure conditions were as follow: 30 psi feed pressure and 10 psi retentate pressure. The samples were concentrated down to 50 mL and the resulting product was harvested. 100 mL of loading buffer 2 was used to rinse the TFF system/membrane and subsequently added to the 50 mL of concentrated product (e.g., final volume of 150 mL).

The following samples were tested in subsequent experiments described below: starting material (e.g., plasma), initial dilution (3.33×dilution), the initial filtrate, the final filtrate, concentrated produce (e.g., 3×concentration of the starting material and 10×concentration of initial dilution), and the final product.

Table 10 shows absorbance data at 280 nM (e.g., protein detection).

TABLE 10
Absorbance (A280) Data

| | | Dilution | % Residual |
|---|---|---|---|
| Sample | A280 | Factor | of Starting |
| Blank | 0.049 | n/a | n/a |
| EV Lyo Buffer | 0.054 | n/a | n/a |
| Starting Plasma | 1.300 | 10 | 100.0% |
| Initial Dilution | 0.437 | 10 | 27.6% |
| Initial Filtrate | 0.812 | n/a | 5.7% |
| Final Filtrate | 1.180 | n/a | 8.6% |
| Concentrated | 0.878 | 40 | 250.0% |
| Final Product | 1.032 | 10 | 74.7% |

TABLE 11
Osmolarity Data

| | Osmolarity |
|---|---|
| Sample | (mOsm) |
| EV Lyo Buffer | 588 |
| Starting Plasma | 282 |
| Initial Dilution | 491 |
| Initial Filtrate | 489 |
| Final Filtrate | 496 |
| Concentrated | 511 |
| Final Product | 552 |

TABLE 11
NT A Summary

| | Mean | | | | % Yield (of |
|---|---|---|---|---|---|
| | Diameter | Concentration | Processing | | Previous |
| Sample/Step | (nm) | (Particles/mL) | Volume (mL) | Total EVs | Sample) |
| Starting (fridge) | 130.2 | 3.45E+13 | 150 | 5.18E+15 | N/A |
| Starting (frozen) | 148.1 | 1.75E+13 | | 2.63E+15 | 50.7% |
| Initial Dilution (frozen) | 140.5 | 4.45E+12 | 500 | 2.23E+15 | 84.8% |
| Final Product (frozen) | 173.9 | 1.01E+13 | 150 | 1.52E+15 | 68.1% |

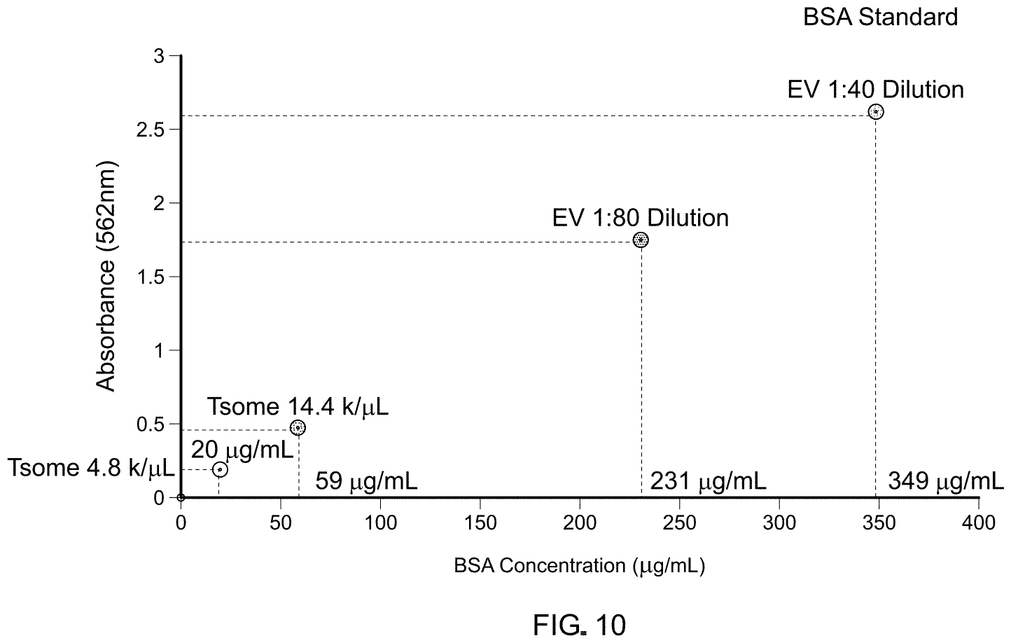

FIG. 10 is a graph showing the results of the extracellular vesicle micro bicinchoninic acid (BCA) assay. Extracellular vesicles and thrombosomes were lysed at 37° C. for 30 minutes in 5% TritonX in PBS buffer. 100 μL of the lysed samples or standards were loaded into 96-well plates and incubated at room temperature for 2 hours and absorbance was measured at 562 nm. The data show that one 1FP extracellular vesicle protein concentration equal 18 mg/mL and 4.8 k/μL thrombosomes=20 μg/mL.

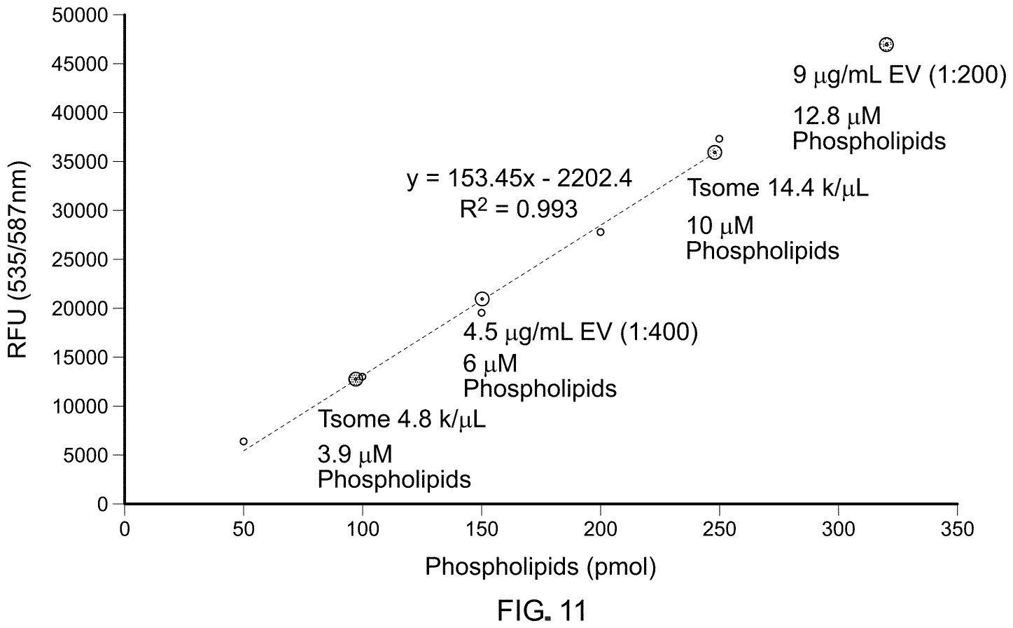

FIG. 11 is a graph showing the results of extracellular vesicle phospholipids in a fluorometric assay. Samples were prepared as follows: 25 μL of sample or a phospholipid standard; 25 μL background control sample; 25 μL reaction mix; and 25 μL of background reaction mix for a total of 100 μL for each sample. All samples were loaded into a 96-well plate and incubated at 25° C. for 30 minutes in the dark. Fluorometric metric measurements were measures at Ex/Em=535/587 nm. The data show that extracellular vesicle phospholipid concentration=2.4 mM and phospholipid concentration at 4.8 k/μL of thrombosomes=3.9 μM.

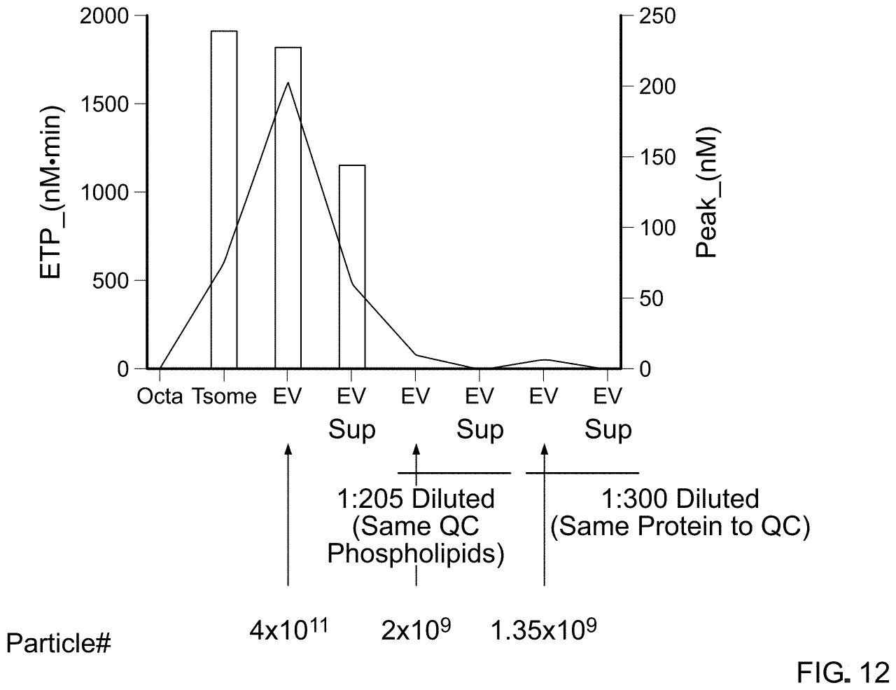

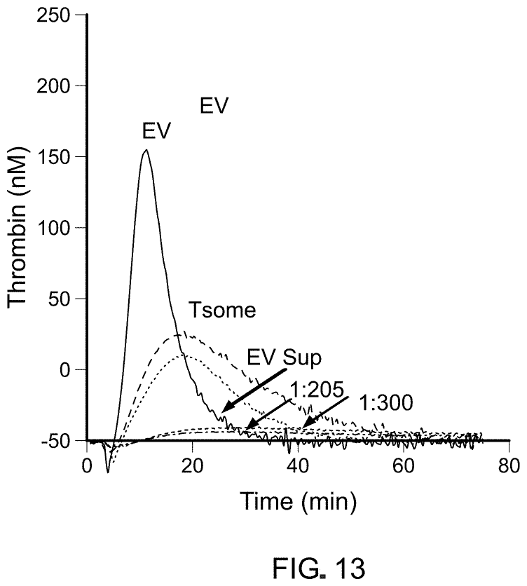

FIGS. 12 and 13 are graphs showing the results of extracellular vesicles ability to generate thrombin in a thrombin generation assay (TGA). Extracellular vesicles were prepared by centrifugation at 100,000 rpm at 4° C. for 1 hour. Samples were prepared as follows: 40 μL of either undiluted or diluted extracellular vesicle supernatant (e.g., post centrifugation); 40 μL of extracellular vesicles; and 40 μL thrombosomes (e.g., control sample). The samples were loaded into a 96-well plate with 40% Octaplas and either a platelet rich plasma reagent or a microparticle reagent (e.g., Stago, Thrombinoscope Reagent: CAT #86222 https://www.thrombinoscope.com/method-products/products/#c4577). Microparticle reagent contains phospholipids only. Tissue Factor bearing microparticles are able to trigger thrombin generation in plasma. Thus, the use of microparticle reagent makes the assay specifically sensitive to Tissue Factor present in circulation on the surface of microparticles. FIG. 12 shows endogenous thrombin potential (ETP), which corresponds to the area for the various samples tested. Extracellular vesicles at a particle number of 4×10¹¹ show similar ETP as compared to thrombosomes. FIG. 13 is a graph showing peak thrombin generation and the ability of extracellular vesicles to generate peak thrombin of about 200 nM at about 10 minutes, demonstrating an increase in the amount of thrombin generated and a decrease in time, relative to thrombosomes.

### Example 10. Extracellular Vesicle Tangential Flow Filtration (TFF)—Frozen Plasma

The starting material from TFF processing began with irradiated, apheresis platelets (6-unit, 4 donor pool). Platelets were aged at 4° C. from day 2 to day 14 without any agitation. The platelets were centrifuged at 2500 g for 20 minutes on day 14. Next, plasma was expressed from the platelet pellet and filtered with 0.45 μm filter flask. 190 mL was then frozen in an ethyl vinyl acetate (EVA) transfer bag at −80° C. for long term storage.

The platelet poor plasma (PPP) thawing cycle: 37° C. for 10 minutes with agitation in a Helmer plasma thawer until room temperature is reached. The thawed PPP was then acidified to pH 6.6-6.8 with ACD, followed by dilution and filtration of the acidified PPP. The PPP was diluted 2:1 with loading buffer 2 as in Example 1 (e.g., PBS (acidified to pH 6.6-6.8 with AC)) and then formulated to 7% sucrose, 2% trehalose (% w/w)) and then filtered with a 0.22 μm filter flask. The filter flask was then washed with 100 mL loading buffer 2 to rinse the filter.

Tangential flow filtration (TFF) was performed with a filter membrane: 500 kDa NMWCO BioMax PES membrane. Pressure conditions were as follows: 30 psi feed and 10 psi retentate pressure. The TFF system was prepared as follows: washed with 1L H₂O, cleaned with 500 mL 0.1 M NaOH at 45° C. for 1 hour, washed again with 1L H₂O, then preconditioned with 50 mL of loading buffer 2 (e.g., PBS (acidified to pH 6.6-6.8 with ADC)) and then formulated to 7% sucrose, 2% trehalose (% w/w)). The diluted and filtered starting material was then transferred into the TFF tank. Next, the product was concentrated to 150 mL, then the volume was brought back up to 500 mL with 350 mL of loading buffer 2 and concentrated again to 50 mL and harvested. 50 mL of loading buffer 2 was used to rinse the TFF system to recoup EVs and was then harvested.

50 mL of harvested, concentrated extracellular vesicles were filtered with a 0.22 μm filter flask followed by 50 mL of TFF system rinse, and finally 50 mL of loading buffer 2 was used to rinse the filter flask resulting in 150 mL of the final product. Next, 100 vials were each filled with 1 mL of the final product for lyophilization and separately 32 tubes were filled with 1 mL of the final product for freezing.

TABLE 12
Osmolarity, A280, and Viscosity Data

| | | | Processing | |
|---|---|---|---|---|
| | Osmolarity | % Residual | Volume | Viscosity @ |
| Sample (fresh) | (mOsm) | A280 | (mL) | 37 C. (cP) |
| Plasma | 282 | 100% | 190 | 1.06 |
| EV Lyo Buffer | 598 | n/a | 760 | .954 |
| | | | (wash) | |
| Initial Dilution | 502 | 33% | 500 | 1.007 |
| Final Product | 609 | 96% | 150 | 1.492 |
| Filtrate (bulk) | 536 | 4% | 800 | .931 |

TABLE 13
NTA and Thrombolux Data

| | NTA - | | | TLUX - | TLUX - |
|---|---|---|---|---|---|
| | Mean | NTA - | Processing | Mean | Scattering |
| | Diameter | Concentration | Volume | Diameter | Intensity |
| Sample | (nm) | (particles/mL) | (mL) | (nm) | (kHz) |
| Initial Dilution (frozen) | 181.8 | 4.7e+12 | 500 | 252.6 | 55 |
| Final Product (frozen) | 171.3 | 2.66e+13 | 150 | 141.4 | 97 |
| Final Product (lyophilized) | 138.2 | 2.11e+13 | | ND | ND |

The data in Table 12 shows that buffer exchange for final product was sufficient and the final product has a 96% residual A280 of starting plasma values.

The data in Table 13 shows an approximate total 0 yield based on the thrombolux assay (e.g., TLUX) is 52.8%, by using scattering intensity. After estimating and accounting for freeze/thaw loss by using historical data, a total 0 yield from thawed plasma to the frozen final product is 73.51.

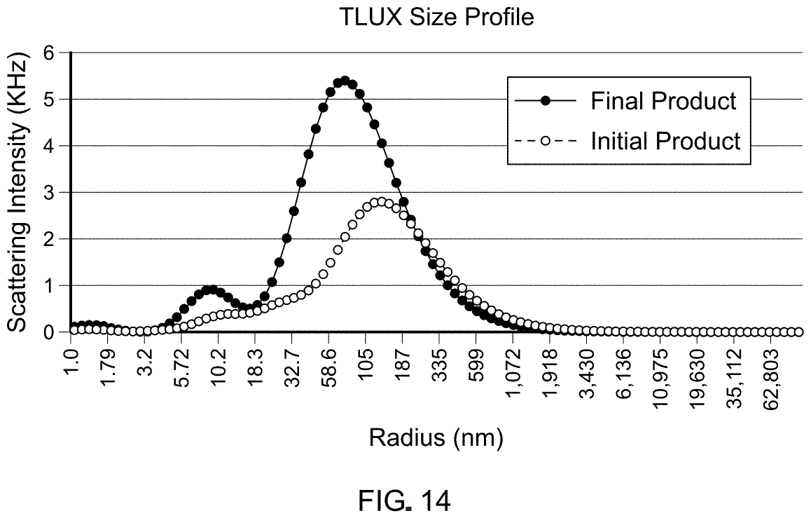

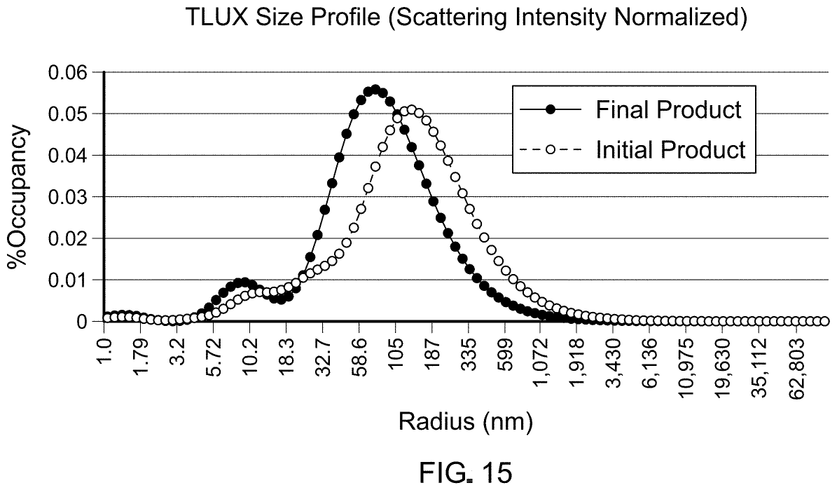

FIGS. 14 and 15 show thrombolux data. FIG. 14 is a graph showing thrombolux (e.g., TLUX) Size Profile showing scattering intensity (kHlz) by radius to demonstrate the size of the extracellular vesicles between the final product and the initial dilution. FIG. 15 is graph showing thrombolux Size Profile (scattering intensity normalized) showing 00 occupancy by radius. The data is summarized in Table 14 below.

TABLE 14

| | | | TLUX - |
|---|---|---|---|
| | Processing | TLUX - Mean | Scattering |
| Sample | Volume (mL) | Diameter (nm) | Intensity (kHz) |
| Initial Dilution | 500 | 252.6 | 55 |
| (fresh) | | | |
| Final Product (fresh) | 150 | 141.4 | 97 |

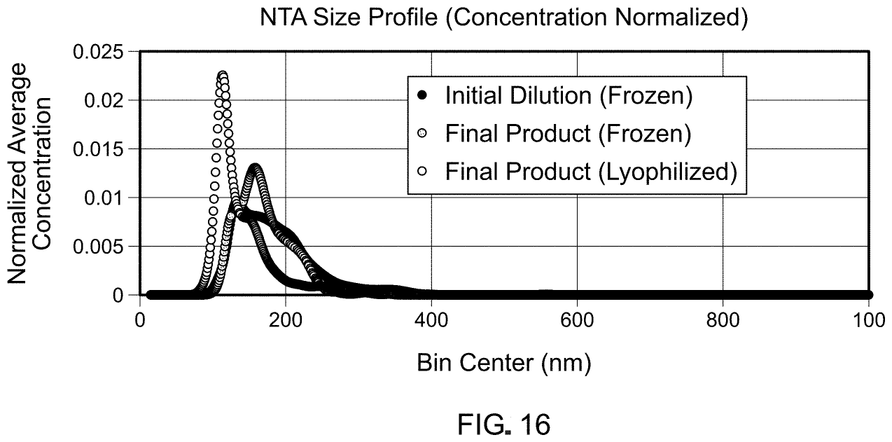

FIG. 16 is a graph showing nano tracking analysis (NTA) size profile where the concentration has been normalized for the following samples: initial dilution (frozen), final product (frozen), and final product (lyophilized). NTA data were obtained using NanoSight instrumentation. The data shown in FIG. 16 is summarized in Table 15 below.

TABLE 15

| | | NTA - | |
|---|---|---|---|
| | NTA - Mean | Concentration | Processing |
| Sample | Diameter (nm) | (particles/mL) | Volume (mL) |
| Initial | 181.8 | 4.7e+12 | 500 |
| Dilution (frozen) | | | |
| Final Product | 171.3 | 2.66e+13 | 150 |
| (frozen) | | | |
| Final Product | 138.2 | 2.11e+13 | |
| (lyophilized) | | | |

### Example 11. Thrombin Generating Assay and Phospholipid Content

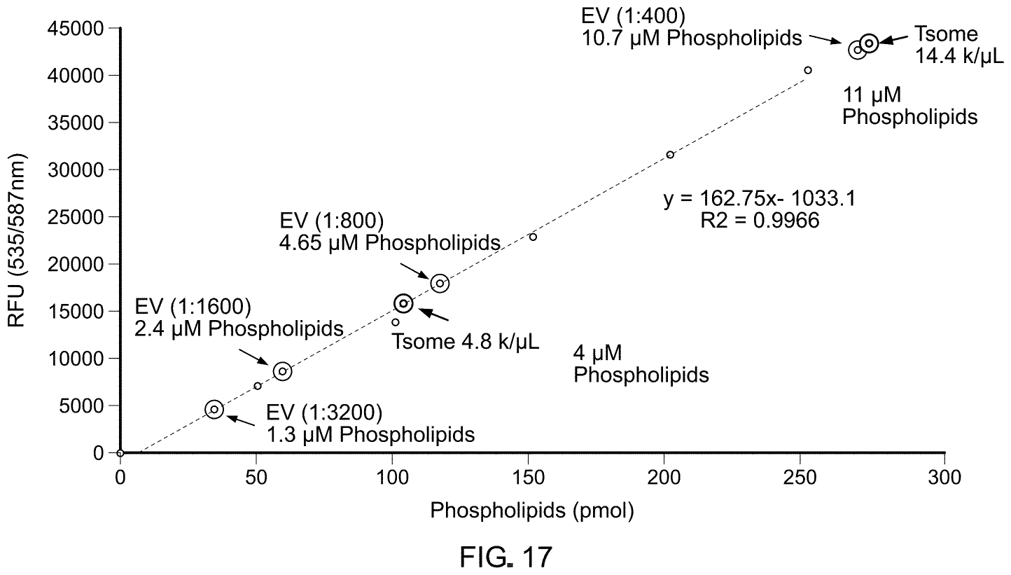

FIG. 17 is a graph showing the concentration of phospholipid content in various samples as measured by a phospholipid fluorometric assay. Samples were prepared as follows: 25 μL of sample or a phospholipid standard; 25 μL background control sample; 25 μL reaction mix; and 25 μL of background reaction mix for a total of 100 μL for each sample. All samples were loaded into a 96-well plate and incubated at 25° C. for 30 minutes in the dark. Fluorometric metric measurements were measures at Ex/Em=535/587 nm. The data show that extracellular vesicle phospholipid concentration=4 mM and phospholipid concentration at 4.8 k/μL of thrombosomes=4.0 μM.

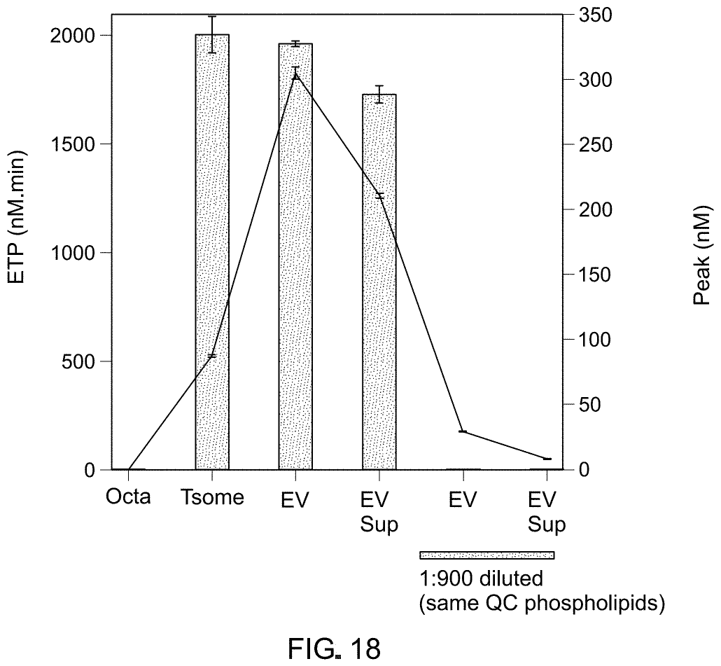

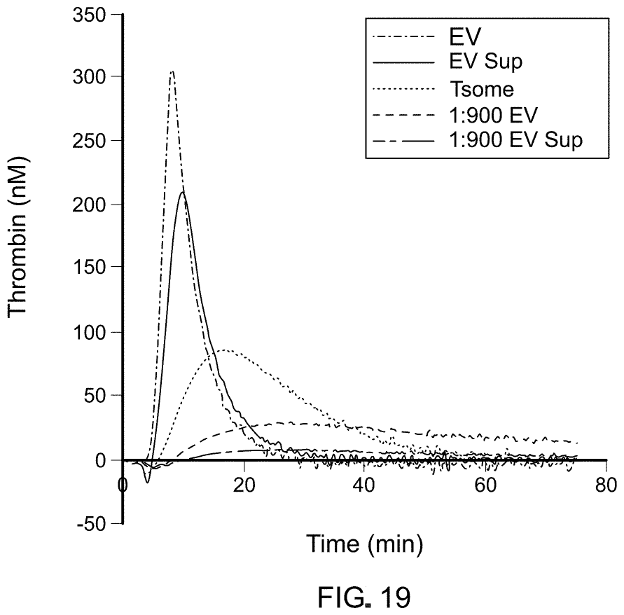

FIGS. 18 and 19 are graphs showing the results of extracellular vesicles ability to generate thrombin in a thrombin generation assay (TGA). Extracellular vesicles were prepared by centrifugation at 100,000 rpm at 4° C. for 1 hour. Samples were prepared as follows: 40 μL of either undiluted or diluted extracellular vesicle supernatant (e.g., post centrifugation); 40 μL of extracellular vesicles; and 40 μL thrombosomes (e.g., control sample). The samples were loaded into a 96-well plate with 40% Octaplas and either a platelet rich plasma reagent or an MP reagent. FIG. 18 shows endogenous thrombin potential (ETP), which corresponds to the area for the various samples tested. Extracellular vesicles show similar ETP as compared to thrombosomes. FIG. 19 is a graph showing peak thrombin generation and the ability of extracellular vesicles to generate peak thrombin of about 300 nM at about 10 minutes, demonstrating an increase in the amount of thrombin generated and a decrease in time, relative to thrombosomes.

### Example 12. Lyophilized Extracellular Vesicles—Tangential Flow Filtration

Fresh platelet poor plasma (PPP) was obtained separated from whole blood in citrate. PPP was substituted with 10% of the following samples: PBS buffer, loading buffer 2 as described in Example 1, or extracellular vesicles (e.g., 450 μL PPP and 50 μL of each sample as described previously). The various samples were run on the T-TAS over the AR chip. Data was collected for each sample until T10 was reached. T10 equals the time to obtain an increase of 10 pKa back pressure in the channel due to clot forming.

Each sample was also tested with Octaplas instead of fresh PPP as a control. For example, Octaplas was substituted with 10% of the following samples: loading buffer 2 or extracellular vesicles (e.g., 450 μL Octaplas and 50 μL of each sample as previously described.

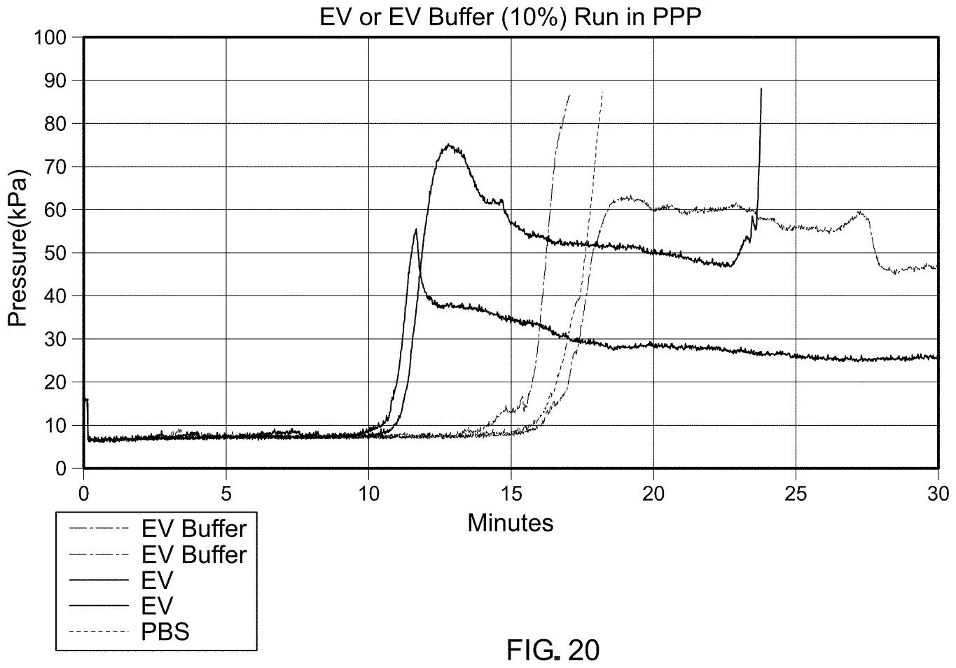

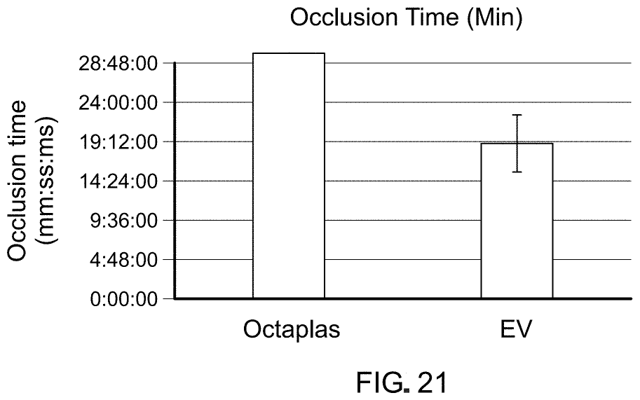

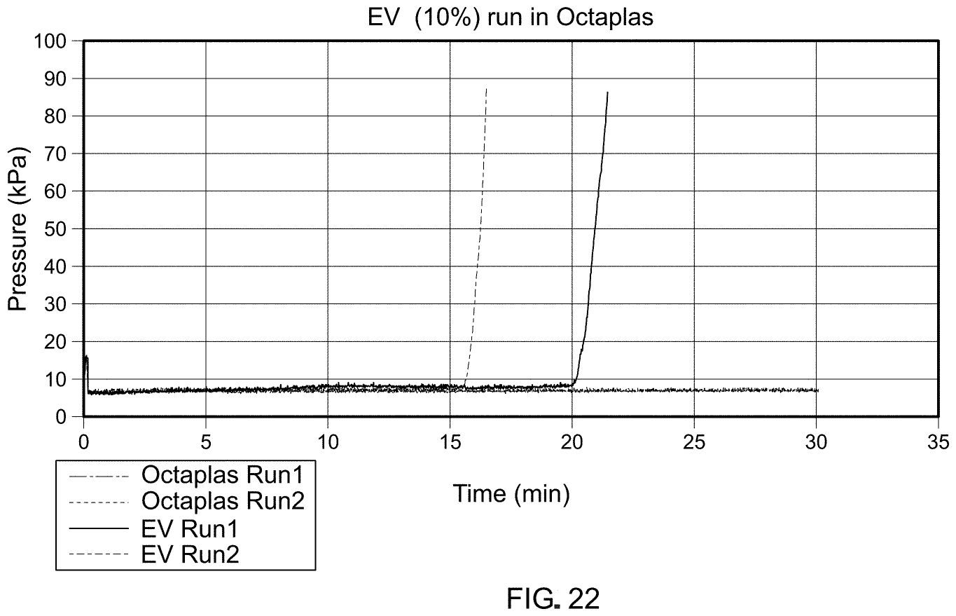

FIG. 20 is a graph showing pressure (kPa) over time. Various samples were tested including extracellular vesicle buffer (e.g., loading buffer 2), extracellular vesicles, and PBS buffer. The data demonstrate that extracellular vesicles reach peak pressure prior to either loading buffer 2 (e.g., EV buffer) or PBS. FIG. 21 is a graph showing occlusion time between Octaplas (positive control) and extracellular vesicles. FIG. 22 is a graph showing extracellular vesicle (10%) run in Octaplas.

### Example 13. Extracellular Vesicles and Surface Markers

Samples as prepared in Example 10 were rehydrated e.g., 1×vial R-EV-21-001 (MFG 8 Feb. 2021). Antibody isotype stains were prepared for the following cell surface markers: CD9, CD41, Lactadherin, CD62, CD42, 9F9. The samples were analyzed on the Novocyte Flow Cytometer with the following parameters: FSC, SSC, FITC, and PECy5; stop conditions: 50,000 events or 50 μl; FSC-H: 350; and flow rate: slow preset.

Gate desired population was controlled first by size, then gated by isotype such that the positivity was 1.3-1.5% in the isotype control. Those gate conditions were applied to all test samples.

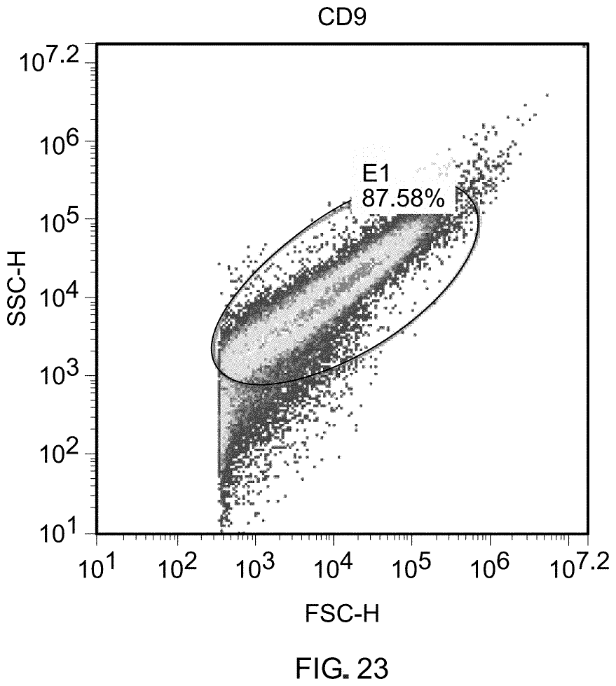

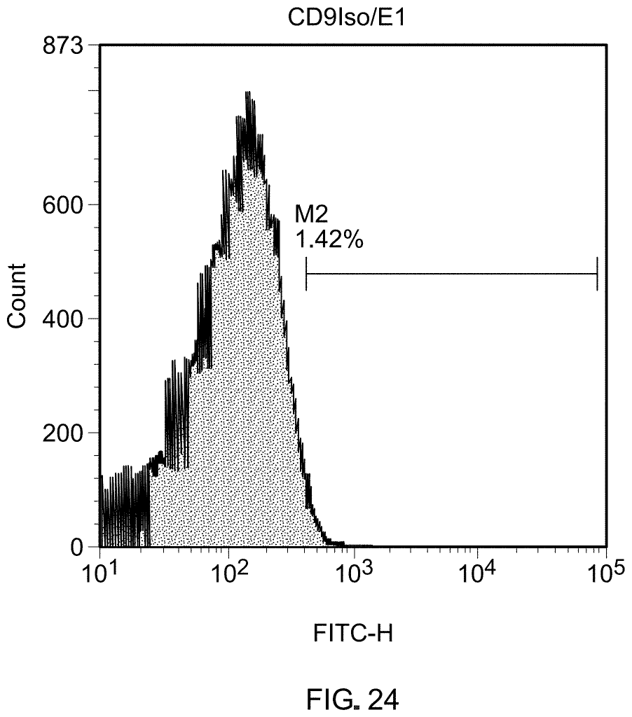

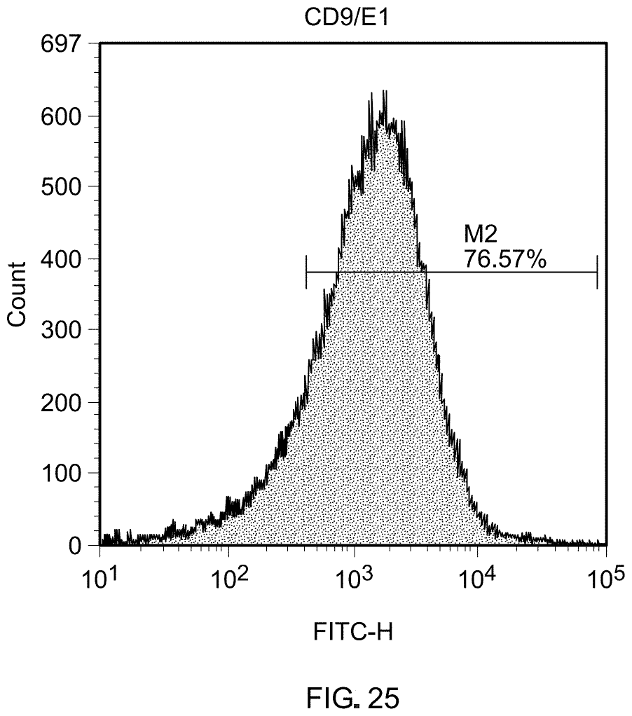

Collectively FIGS. 23-25 show a gating example with CD9 expression. For example, FIG. 23 shows CD9 gating as SSC-H by FSC-H, FIG. 24 shows CD9 isotype expression, and FIG. 25 shows CD9 expression.

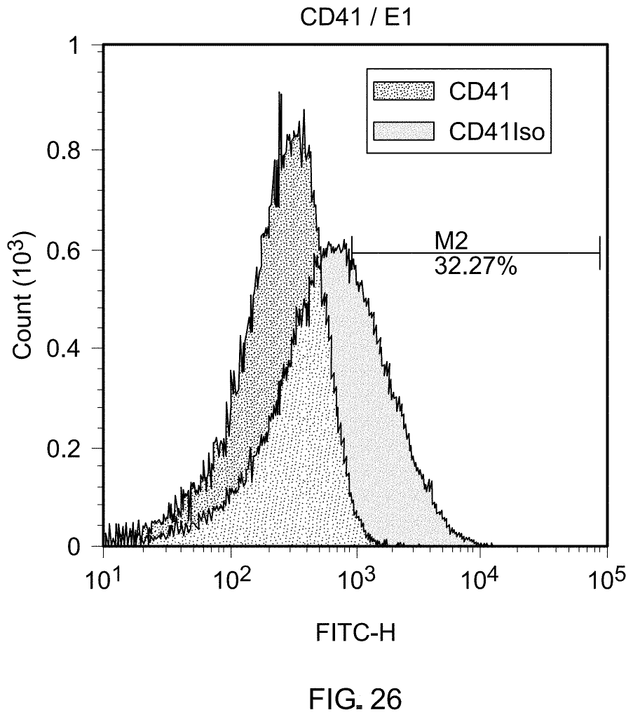

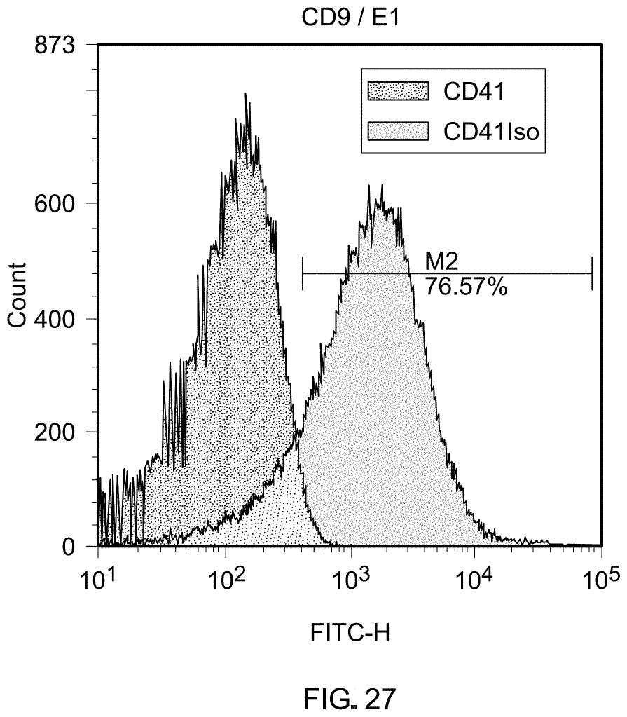

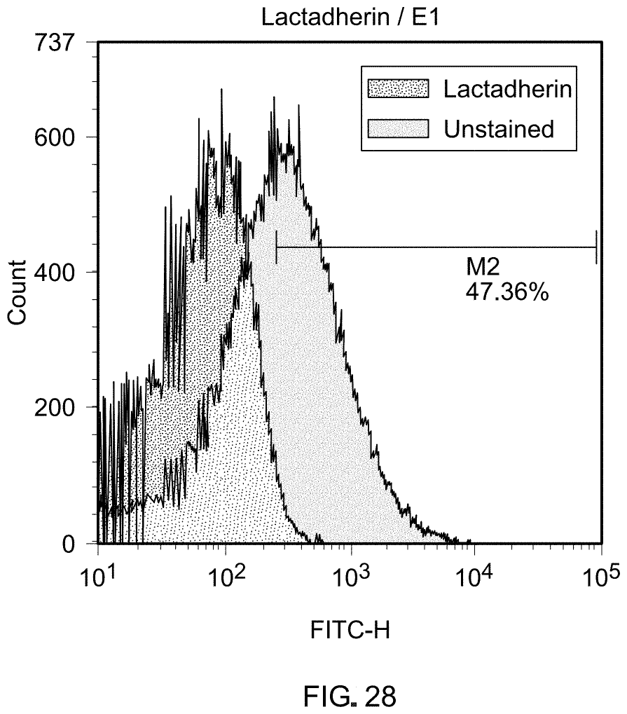

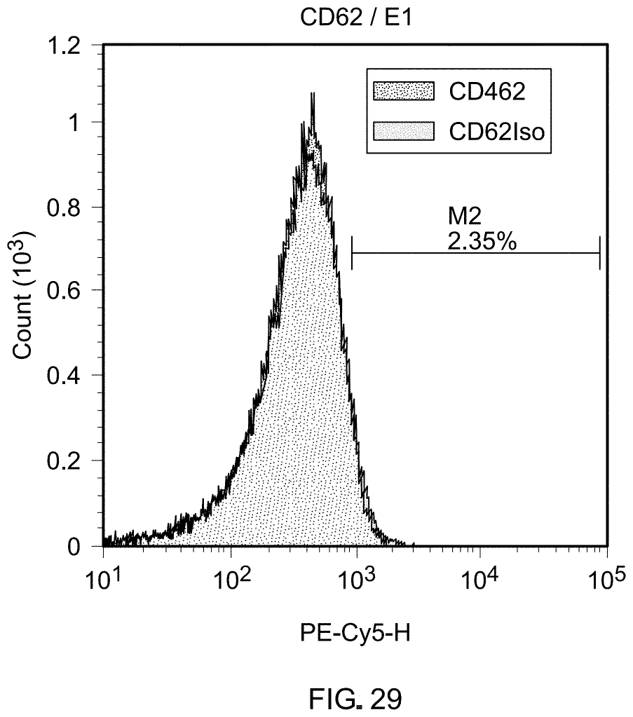

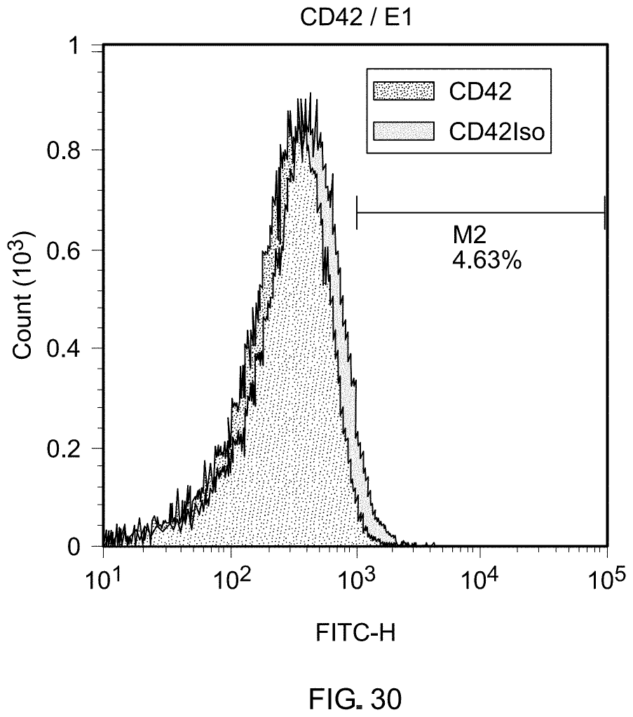

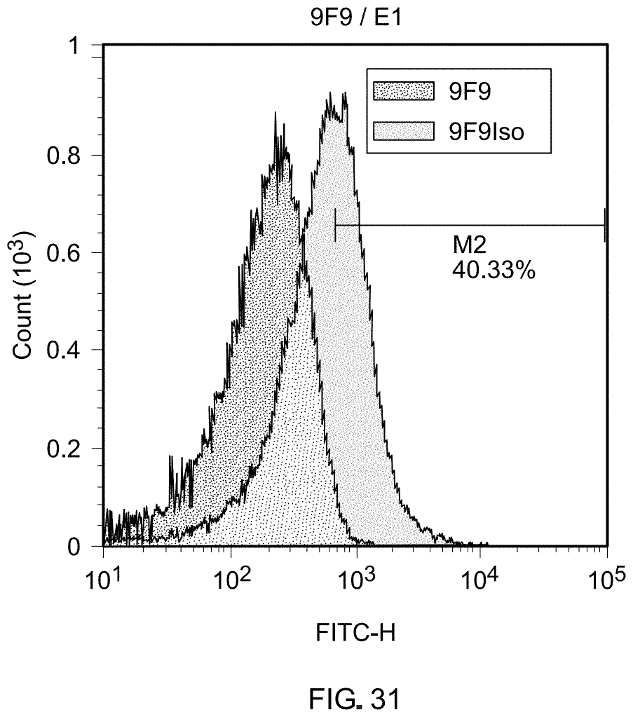

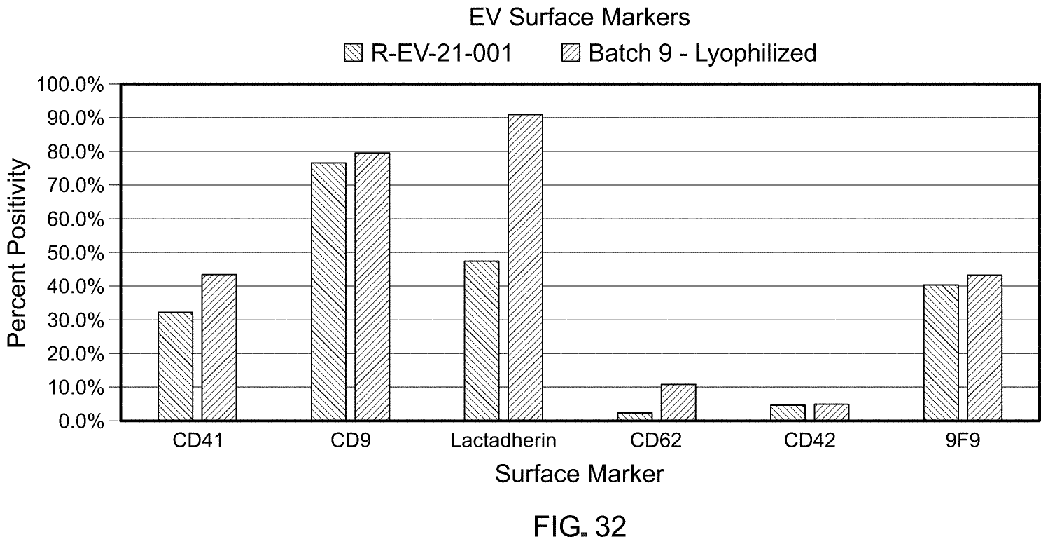

Collectively, the Novocyte Flow Cytometer results for CD41, CD9, Lactadherin, CD62, CD42, and 9F9 are show in FIGS. 26-31, respectively. FIG. 32 is a graph summarizing the results of all surface markers tested. CD41, CD9, CD62, CD42, and 9F9 were compared to their respective isotypes. Lactadherin was compared to unstained samples. FIG. 32 also shows the percent positivity of various cell surface makers in FIGS. 26-31 (e.g., CD41, CD9, Lactaherin, CD62, CD42, and 9F9) between frozen (e.g., R-EV-21-001) extracellular vesicles and lyophilized (e.g., Batch 9) extracellular vesicles.

### Embodiments

Embodiment 1 is a method of preparing stabilized platelet-derived extracellular vesicles, the method comprising: generating a population of extracellular vesicles from platelets by allowing the platelets to shed a population of extracellular vesicles over a period of time in the range of approximately 2 days to approximately 21 days; isolating the population of extracellular vesicles; and stabilizing the populations of extracellular vesicles with a loading buffer, to form comprising a salt, a base, a loading agent, and optionally at least one organic solvent to form stabilized platelet derived extracellular vesicles.

Embodiment 2 is a method of preparing stabilized platelet-derived extracellular vesicles, the method comprising: contacting platelets with a stimulating agent to generate a population of extracellular vesicles; isolating the population of extracellular vesicles; and stabilizing the population of extracellular vesicles with a loading buffer comprising a salt, a base, a loading agent, and optionally at least one organic solvent, to form stabilized platelet-derived extracellular vesicles.

Embodiment 3 is a method of preparing stabilized platelet-derived extracellular vesicles, the method comprising: contacting platelets with a stimulating agent to generate a population of extracellular vesicles; isolating the population of extracellular vesicles; and stabilizing the population of extracellular vesicles with a loading buffer comprising a salt, a base, a saccharide, and at least one organic solvent, to form stabilized platelet-derived extracellular vesicles. Embodiment 4 is the method of embodiment 2 or 3, wherein the stimulating agent comprises one of a chemical stimulating agent, a mechanical stimulating agent, or combinations thereof.

Embodiment 5 is the method of any one of embodiments 2-4, wherein the platelets are contacted with a stimulating agent for a period of time in the range of approximately 5 minutes to approximately 24 hours.

Embodiment 6 is the method of embodiment 4, wherein the chemical stimulating agent comprises a calcium ionophore, collagen, thrombin, a hypertonic solution, or combinations thereof.

Embodiment 7 is the method of embodiment 4, wherein the mechanical stimulating agent comprises sonication.

Embodiment 8 is the method of embodiment 2 or 3, wherein one or more platelet-derived extracellular vesicles of the population of extracellular vesicles comprises an exosome. Embodiment 9 is the method of embodiment 8, wherein the exosome is in the range of approximately 40 nm to approximately 200 nm in diameter.

Embodiment 10 is the method of embodiment 2 or 3, wherein one or more platelet-derived extracellular vesicles of the population of extracellular vesicles comprises a microvesicle.

Embodiment 11 is the method of embodiment 10, wherein the microvesicle is in the range of approximately 200 nm to approximately 450 nm.

Embodiment 12 is the method of any one of the preceding embodiments, wherein the method further comprises applying ultracentrifugation to the population of extracellular vesicles.

Embodiment 13 is the method of any one of embodiments 1-11, wherein the method further comprises applying filtration to the population of extracellular vesicles.

Embodiment 14 is the method of any one of the preceding embodiments, wherein the loading buffer comprises a stabilization agent, wherein the stabilization agent is a monosaccharide, a disaccharide, a synthetic polymer of a disaccharide, or combinations thereof.

Embodiment 15 is the method of any one of the preceding embodiments, wherein the loading buffer comprises the stabilization agent, wherein the stabilization agent is sucrose, polysucrose, maltose, trehalose, glucose, mannose, or xylose.

Embodiment 16 is the method of any one of embodiments 1-15, wherein the loading buffer comprises the stabilization agent, wherein the stabilization agent comprises sucrose and trehalose.

Embodiment 17 is the method of any one of embodiments 1-15, wherein the loading buffer comprises the stabilization agent, wherein the stabilization agent comprises polysucrose and trehalose.

Embodiment 18 is the method of embodiment 16 or 17, wherein the trehalose is present in the range of approximately 1% (w/v) to approximately 3% (w/v).

Embodiment 19 is the method of embodiments 16 or 18, wherein sucrose is present in the range of approximately 5% (w/v) to approximately 10% (w/v).

Embodiment 20 is the method of embodiment 17 or 18, wherein the polysucrose is present in the range of approximately 5% (w/v) to approximately 10% (w/v).

Embodiment 21 is the method of any one of the preceding embodiments, wherein the loading buffer comprises PBS and 7% (w/v) sucrose and 2% (w/v) trehalose.

Embodiment 22 is the method of any one of the preceding embodiments, wherein the loading buffer comprises PBS and 7% (w/v) polysucrose and 2% (w/v) trehalose.

Embodiment 23 is the method of any one of the preceding embodiments, wherein the loading buffer further comprises one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

Embodiment 24 is the method of embodiments 2-23, wherein the platelets are isolated prior to the contacting step.

Embodiment 25 is the method of embodiments 2-23, wherein the platelets are pooled from a plurality of donors prior to the contacting step.

Embodiment 26 is the method of any one of the preceding embodiments, wherein the stabilized platelet-derived extracellular vesicles are generated from the group consisting of fresh platelets, stored platelets, frozen platelets, freeze-dried platelets, thrombosomes, and any combination thereof.

Embodiment 27 is the method of any one of the preceding embodiments, further comprising cold storing, cryopreserving, freeze-drying, drying, thawing, rehydrating, or combinations thereof the stabilized platelet-derived extracellular vesicles.

Embodiment 28 is the method of embodiment 27, wherein the drying step comprises freeze-drying the stabilized platelet-derived extracellular vesicles.

Embodiment 29 is the method of embodiment 27 or 28, further comprising rehydrating the stabilized platelet-derived extracellular vesicle.

Embodiment 30 is the method of any one of the preceding embodiments, wherein the platelets are further contacted with an imaging agent, wherein the stabilized platelet derived extracellular vesicle is loaded with the imaging agent.

Embodiment 31 is stabilized platelet-derived extracellular vesicles prepared by the method of any one of the preceding embodiments.

Embodiment 32 is a composition comprising stabilized platelet derived extracellular vesicles generated by contacting platelets with a stimulating agent comprising at least about 50% stabilized platelet derived extracellular vesicle by mass.

Embodiment 33 is the composition of embodiment 32, wherein the stabilized platelet-derived extracellular vesicles are generated according to the method of any of embodiments 1-30.

Embodiment 34 is a composition comprising stabilized platelet derived extracellular vesicles generated by contacting platelets with a stimulating agent comprising at least about 75% stabilized platelet derived extracellular vesicles by mass.

Embodiment 35 is a composition comprising stabilized platelet derived extracellular vesicles generated by contacting platelets with a stimulating agent comprising at least about 80% stabilized platelet derived extracellular vesicles by mass.

Embodiment 36 is a composition comprising stabilized platelet derived extracellular vesicles generated by contacting platelets with a stimulating agent comprising at least about 90% stabilized platelet derived extracellular vesicles by mass.

Embodiment 37 is a method of treating bleeding in a subject in need thereof, the method comprising: administering a therapeutically effective amount of stabilized platelet-derived extracellular vesicles generated by contacting platelets with a stimulating agent to the subject in need thereof.

Embodiment 38 is a method of treating a wound in a subject in need thereof, the method comprising: administering a therapeutically effective amount of stabilized platelet-derived extracellular vesicles generated by contacting platelets with a stimulating agent to the subject in need thereof.

Embodiment 39 are stabilized platelet-derived extracellular vesicles prepared by the method of any one of the preceding embodiments for treating or ameliorating regenerative medicine.

Embodiment 40 is a regenerative medicine method comprising administering stabilized platelet-derived extracellular vesicles prepared by the method of any one of the preceding embodiments to a subject in need thereof.

Embodiment 41 is a regenerative medicine method comprising administering stabilized platelet-derived extracellular vesicles to a subject in need thereof, wherein the stabilized platelet-derived extracellular vesicles comprise one or more growth factors, one or more cytokines, one or more chemokines, and combinations thereof.

Embodiment 42 is the method of embodiment 41, wherein the one or more growth factors comprises platelet factor 4.

Embodiment 43 is a method of preparing drug-loaded stabilized platelet-derived extracellular vesicles, the method comprising: contacting platelets with a drug and a loading buffer comprising a salt, a base, a loading agent, and optionally at least one organic solvent to form drug-loaded platelets; contacting the drug-loaded platelets with a stimulating agent to generate a population of drug-loaded extracellular vesicles; isolating the population of drug-loaded extracellular vesicles; and stabilizing the population of drug-loaded extracellular vesicles with the loading buffer, to form drug-loaded extracellular vesicles.

Embodiment 44 is a method of preparing drug-loaded stabilized platelet-derived extracellular vesicles, the method comprising: contacting platelets with a drug and a loading buffer comprising a salt, a base, a loading agent, and optionally at least one organic solvent to form drug-loaded platelets; generating a population of drug-loaded extracellular vesicles from the drug-loaded platelets by allowing the drug-loaded platelets to shed the population of extracellular vesicles over a period of time in the range of approximately 2 days to approximately 21 days; isolating the population of drug loaded extracellular vesicles; and stabilizing the population of drug-loaded extracellular vesicles with the loading buffer, to form the drug-loaded extracellular vesicles.

Embodiment 45 is the method of embodiment 43 or 44, wherein the drug and the loading buffer are contacted with the platelets sequentially in either order, or concurrently.

Embodiment 46 is the method of any one of embodiments 43-45, wherein the loading buffer comprises either sucrose or polysucrose and trehalose.

Embodiment 47 is the method of embodiment 43 or 44, wherein the loading buffer comprises trehalose in the range from about 0.1% (w/v) to about 5% (w/v).

Embodiment 48 is the method of embodiment 43 or 44, wherein the loading buffer comprises either polysucrose or sucrose in the range from about 2% (w/v) to about 10% (w/v).

Embodiment 49 is the method of any one of embodiments 43-48, wherein the drug-loaded stabilized platelet-derived extracellular vesicles treat a disease in a subject in need thereof.

Embodiment 50 is the method of embodiment 49, wherein the disease is cancer.

Embodiment 51 is the method of any one of embodiments 43-50, further comprising cold storing, cryopreserving, freeze-drying, thawing, rehydrating, and combinations thereof the drug-loaded stabilized platelet-derived extracellular vesicles.

Embodiment 52 is the method of any one of embodiments 43-51, wherein the drug-loaded stabilized platelet-derived extracellular vesicles are generated from the group consisting of drug-loaded fresh platelets, drug-loaded stored platelets, drug-loaded frozen platelets, drug-loaded freeze-dried platelets, drug-loaded thrombosomes, and any combination thereof.

What is claimed is:

1. A method of preparing stabilized platelet-derived extracellular vesicles, the method comprising:
generating a population of extracellular vesicles from platelets by allowing the platelets to shed a population of extracellular vesicles over a period of time in the range of approximately 2 days to approximately 21 days;
isolating the population of extracellular vesicles; and
stabilizing the populations of extracellular vesicles with a loading buffer comprising a salt, a base, a loading agent, and optionally at least one organic solvent, to form stabilized platelet-derived extracellular vesicles.

2. A method of preparing stabilized platelet-derived extracellular vesicles, the method comprising:
contacting platelets with a stimulating agent to generate a population of extracellular vesicles;
isolating the population of extracellular vesicles; and
stabilizing the population of extracellular vesicles with a loading buffer comprising a salt, a base, at least one organic solvent, and optionally a saccharide to form stabilized platelet-derived extracellular vesicles.

3. The method of claim 2, wherein the stimulating agent comprises one of a chemical stimulating agent, a mechanical stimulating agent, or combinations thereof, and wherein the platelets are contacted with a stimulating agent for a period of time in the range of approximately 5 minutes to approximately 24 hours.

4. (canceled)

5. The method of claim 3, wherein the chemical stimulating agent comprises a calcium ionophore, collagen, thrombin, a hypertonic solution, or combinations thereof, and wherein the mechanical stimulating agent comprises sonication.

6. (canceled)

7. The method of claim 2, wherein one or more stabilized platelet-derived extracellular vesicles comprises an exosome, wherein the exosome is in the range of approximately 40 nm to approximately 200 nm in diameter.

8. The method of claim 2, wherein one or more stabilized platelet-derived extracellular vesicles comprises a microvesicle, wherein the microvesicle is in the range of approximately 200 nm to approximately 450 nm.

9. The method of claim 2, wherein the method further comprises ultracentrifuging to the population of extracellular vesicles.

10. The method of claim 2, wherein the method further comprises filtering the population of extracellular vesicles.

11. The method of claim 2, wherein the loading buffer comprises a stabilization agent, wherein the stabilization agent is a monosaccharide, a disaccharide, a synthetic polymer of a disaccharide, or combinations thereof.

12. The method of claim 11, wherein the stabilization agent is sucrose, polysucrose, maltose, trehalose, glucose, mannose, or xylose.

13. The method of any one of claim 11, wherein the stabilization agent comprises sucrose and trehalose.

14. The method of any one of claim 11, wherein the stabilization agent comprises polysucrose and trehalose.

15. The method of claim 12, wherein the trehalose is present in the range of approximately 1% (w/v) to approximately 3% (w/v), the sucrose present in the range of approximately 5% (w/v) to approximately 10% (w/v), and the polysucrose is present in the range of approximately 5% (w/v) to approximately 10% (w/v).

16. The method of claim 2, wherein the loading buffer comprises PBS and:
(i) either 7% (w/v) sucrose or 7% (w/v) polysucrose;
(ii) 2% (w/v) trehalose; and optionally
wherein the loading buffer further comprises one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

17. (canceled)

18. The method of claim 2, wherein the platelets are isolated prior to the contacting step and optionally, wherein the platelets are pooled from a plurality of donors.

19. (canceled)

20. The method of claim 2, wherein the stabilized platelet-derived extracellular vesicles are generated from the group consisting of fresh platelets, stored platelets, frozen platelets, freeze-dried platelets, thrombosomes, and any combination thereof.

21. The method of claim 2, further comprising cold storing, cryopreserving, freeze-drying, drying, thawing, rehydrating, or combinations thereof the stabilized platelet-derived extracellular vesicles.

22. The method of claim 21, wherein the drying step comprises freeze-drying the stabilized platelet-derived extracellular vesicles.

23. The method of claim 22, further comprising rehydrating the stabilized platelet-derived extracellular vesicle.

24. The method of claim 2, wherein the platelets are further contacted with an imaging agent, and wherein the stabilized platelet derived extracellular vesicle is loaded with the imaging agent.
